SEQUENCE LISTING (29 sequences)

SEQ ID NO: 1 - protein, 1174 aa, Nocardia iowensis
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 2 - protein, 1162 aa, Mycobacterium phlei
MASESRDVRLQRRIAELYDTDPQFAAARPDEAVARAVNAPGLTLSQVIRTVLDNYADRPALGYRAVEFAADPASARTVARLLPRFDTITYRELGERIDATTAALGHDGLRPGERVAILGFSSVDYTTIDIAAFNLGAVSVPLQTSAPPSQLRPMTAETEPAVIAASVDFLDDALELIRTGHAPRRLVVFDFHPEIDDHRDALAAATTGLADTAMTVETLDSLLTRGRTLPAPTGYRDRDDDLALLIYTSGSTGAPKGAMYQRRMVTNMWRRATTAIWGGKEAAPWLTLNFMPMSHVMGRGILSTTLCSGGTAYFAARSDLSTLLEDLALVRPTQLTFVPRIWEMIFQEYQRDVARRPEAEVLADLREHRLGGRFLAAMTGSAPMSPEMTAFAESVLDLHLVDGYGSTEAGSIVLDGQVLRPPVLDYKLVDVPELGYFSTDRPYPRGELLVKTELMFPGYYKRPDITAEVFDEDGYYRTGDIVAELGPDRLAYVDRRNNVLKLSQGEFVTVSKLEAAFAASPLVRQIYIYGNSAHPYLLAVVVPTEDALTRYDAATLKTAISESLQDVGRAAGLQSYEIPRDFLVETTPFTLENGLLTGLRKLARPKLKEHYGERLEQLYTELADGQAEELKELRTHGAQQPTLTTVSRAATALLGTASAEVRPDAHFTDLGGDSLSALTFGNLLGEIYAVEVPVGVIVSPANDLAAIADYIDTARRPGDGRPTFAGVHGDDAAEVHARDLTLDRFLDDATLAAATALPGPAPEIRTVLLTGATGFLGRYLALEWLERMAMVGGTLICLVRGRDDAAARARLDQIFDSGDPELLRHYRELADRHLEVIAGDKSDAVLGLDRRTWQRLADTVDLIVDPAALVNHVLPYRELFGPNVVGTAELIRLALTGRLKPYLYTSTIAVGAGIAPGQFTEDADIRQISATRTLDDSYANGYATSKWAGEVLLREAHDLCGLPVAVFRCDMILADTSYAGQLNVPDMFTRLILSLVATGIAPLSFYELDAAGHRQRAHYDGLPVEFVAEAVSALGLDVAEDGGFATYHVMNPYDDGIGLDEFVDWLTGAGYPIERVNDYGTWFQRFETAIRGLPERQRQASLLPLLHNYQRPQPPIRGSAAPTDRFRSAVQDAKIGPDKDIPHITPEVIVKYVTDLRLLGLL

SEQ ID NO: 3 - protein, 1168 aa, Mycobacterium smegmatis
MTIETREDRFNRRIDHLFETDPQFAAARPDEAISAAAADPELRLPAAVKQILAGYADRPALGKRAVEFVTDEEGRTTAKLLPRFDTITYRQLAGRIQAVTNAWHNHPVNAGDRVAILGFTSVDYTTIDIALLELGAVSVPLQTSAPVAQLQPIVAETEPKVIASSVDFLADAVALVESGPAPSRLVVFDYSHEVDDQREAFEAAKGKLAGTGVVVETITDALDRGRSLADAPLYVPDEADPLTLLIYTSGSTGTPKGAMYPESKTATMWQAGSKARWDETLGVMPSITLNFMPMSHVMGRGILCSTLASGGTAYFAARSDLSTFLEDLALVRPTQLNFVPRIWDMLFQEYQSRLDNRRAEGSEDRAEAAVLEEVRTQLLGGRFVSALTGSAPISAEMKSWVEDLLDMHLLEGYGSTEAGAVFIDGQIQRPPVIDYKLVDVPDLGYFATDRPYPRGELLVKSEQMFPGYYKRPEITAEMFDEDGYYRTGDIVAELGPDHLEYLDRRNNVLKLSQGEFVTVSKLEAVFGDSPLVRQIYVYGNSARSYLLAVVVPTEEALSRWDGDELKSRISDSLQDAARAAGLQSYEIPRDFLVETTPFTLENGLLTGIRKLARPKLKAHYGERLEQLYTDLAEGQANELRELRRNGADRPVVETVSRAAVALLGASVTDLRSDAHFTDLGGDSLSALSFSNLLHEIFDVDVPVGVIVSPATDLAGVAAYIEGELRGSKRPTYASVHGRDATEVRARDLALGKFIDAKTLSAAPGLPRSGTEIRTVLLTGATGFLGRYLALEWLERMDLVDGKVICLVRARSDDEARARLDATFDTGDATLLEHYRALAADHLEVIAGDKGEADLGLDHDTWQRLADTVDLIVDPAALVNHVLPYSQMFGPNALGTAELIRIALTTTIKPYVYVSTIGVGQGISPEAFVEDADIREISATRRVDDSYANGYGNSKWAGEVLLREAHDWCGLPVSVFRCDMILADTTYSGQLNLPDMFTRLMLSLVATGIAPGSFYELDADGNRQRAHYDGLPVEFIAEAISTIGSQVTDGFETFHVMNPYDDGIGLDEYVDWLIEAGYPVHRVDDYATWLSRFETALRALPERQRQASLLPLLHNYQQPSPPVCGAMAPTDRFRAAVQDAKIGPDKDIPHVTADVIVKYISNLQMLGLL

SEQ ID NO: 4 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 5 - protein, 1156 aa, Nocardia otitidiscaviarum
MLDDARAERRERRIADALADDQVREAVADAAVSESVRRVEVRLARIVDAVMSGYGDRAALAWRRSELVDGAVRLLPEYSTMTYRELWRRAGAVAAEWGADAESPVRAGDFVCTLGFTSPDYTVVDLALVRLGAVAVPLQASASVAQWRSIMAETEPRMLATSAETLPAAVEAVLGGFAPRRVLVFDYRPELEAHRSAVDSARERLAEVGCRVATVADAVDRGASLPAPLCIPSDRERLALLIYTSGSTGAPKGAMYTDRLAAGLWLSANDIRVPALTMNYMPLSHVAGRMSLYGTLMRGGTAYFAAASDMSTLLDDFGLARPTELFLVPRVCELLHQRYQSELDRRVLAGDDVETTATDVKAELRERVLGGRYLTALSGSAPLAAEMKTFMESLLDDELHDGYGSTEAGGSVLLDNRIKRPPVLDYRLVDVPELGYFRTDKPHPRGELLLKTESMFPGYYKRPEITAEMFDADGFYRTGDVVAELGPDQLVYVDRRNNVLKLSQGEFVTVAALEAVYATSPLIRQIFVYGSSERAYLLAVVVPTDEVLALPPARARAEVGESLHRIAKESGLQPYEIPREVLIESEPFTIDNGLLSGIGKLLRPKLKEHYGEQLERLYTELAQQREDELTTLRRGAHERPVLETVTRAAGAVLDLAAGEVSPDAHFADLGGDSLSALSFSTLLRDIFGVEVPVGVIVGPATDLTRIAEYLVAERDSGTRPTAATVHGDDGLLRADDLTLEAFLDPGTLAAATRLSAPTDPPRTVLLTGANGYLGRFLALEWLQRLDVSGGTLICLVRGSDADAARRRLDAVFATGDPELEAHYRDLAGRRLRVLAGDIGEPNLGLSEQDWRDLAESVDVIVHPAALVNHVLPYAQLFGPNVVGTAEVIRLALTARLKPVTYLSTVAVSAGIDPETFTEDGDIREISPVRRLDDGYANGYGNSKWAGEVLLRAAHDRFGLPVAVFRSDMILAHSHYAGQLNVPDMFTRLLLSVLATGLAPGSFHDAHGERHRAHYDGLPADFTAAAVTTLGAQATSGFDTYDVLNPHDDGLSLDTFIDWLIDSGHPIDRIDDYADWFTRFETAVRALPEPQRQHSLLPLLHAYRHPTPPLRGAALPAKHFRAAVQQAKLGPDADIPHLTRELIEKYVSDLRLLGLLG

SEQ ID NO: 6 - protein, 1105 aa, Tsukamurella paurometabola
MSIETVQNGVPAEGSVPPADQQTERLPQVIARIFAQFADRPAFATREAGPGTPYATVSYREIWRRVTALVASWQSEVAPGDFVAILGFTSSDFVTVDLATTLLGAPNVPLQAGAPAARIATILDETRPKILAVSADQVDLAQEALAESAATPRVVVFDGERDGYEGIEADILSGSALPAPEFFAPEPGTDPLVTLIYTSGSTGTPKGAMYTEQLVRDAWLKVDSIVDIDMPAESLLHFLPMSHMYGRNWLIAGLASGGTGYFAGASDMSTLFDDLAAARPTAIGLVPRVCELIHQRYLAVEADTDAETARVELRDRVLGGRLQAAMCGSAALSSELQTFMEWLLGIDIQIGYGSTEAGGVIRDGVVVRPPVTEYKLIDVPELGYFVTDSPHPRGELLVKSTQLIPGYYNSDKRIRDDEGFYRTGDVMAELGPDRLEYVDRRSNVIKLAQGEFVPIAQLEAIYAAGPDVHQIFLYGTSERSYLIGVVVPAPGPDGETDAQTRTRVLDGLAAIARENDLAAYEVPRDVLIERDPFSQENGLRSGIGKLVRPALIARYGDRLHDLYAQADTRQREGLRALDASGPIIDTVLGAAALTLGADIADFDADTRFGDLGGDSLSALSLATTLEGLYDVPVPVQTIVGPTATLGGVARHIEKARSGGVAAPTADSVHGVGASVARATDLTLEKFIDPELLALAPTLPAATGEPNTVLLTGSTGYLGRFLLLDWLRRVAPHGGTVIALVRGADADDARRRVTAAIGDSDPDLTQEFTSLAEHHLHVIAGDFGSPALGLDDATWSDLAGRVDHVVHCGALVNHVLPYDQLFGPNVVATGEVVRLALTTRRKSVDYVSTVAVVPQDDGRVLVEDDDVRELGAERRIGADAYANGYAVSKWAGEVLLHEAADLADLPVRVFRSDMILAHSRFHGQFNEVDQFTRLLLSIAETGLAPASFYTPDPSGHRPHYDGLPVDFTAEAITTLSAAGRSGYRTFHVLNANDDGVSLDSFVDWIAASGRSIERIDDYDTWFARFEQALQQLPDEARQRSVLPLLHAVREPAPAAGTSALSVDRFRGAVRETGVGPGDIPVLDRALIEKYLRDFETAGWLAPGARD

SEQ ID NO: 7 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 8 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 9 - protein, 440 aa, Alcaligenes sp.
  source 1..440: note = Alcaligenes denitrificans
MSAAKLPDLSHLWMPFTANRQFKANPRLLASAKGMYYTSFDGRQILDGTAGLWCVNAGHCREEIVSAIASQAGVMDYAPGFQLGHPLAFEAATAVAGLMPQGLDRVFFTNSGSESVDTALKIALAYHRARGEAQRTRLIGRERGYHGVGFGGISVGGISPNRKTFSGALLPAVDHLPHTHSLEHNAFTRGQPEWGAHLADELERIIALHDASTIAAVIVEPMAGSTGVLVPPKGYLEKLREITARHGILLIFDEVITAYGRLGEATAAAYFGVTPDLITMAKGVSNAAVPAGAVAVRREVHDAIVNGPQGGIEFFHGYTYSAHPLAAAAVLATLDIYRREDLFARARKLSAAFEEAAHSLKGAPHVIDVRNIGLVAGIELSPREGAPGARAAEAFQKCFDTGLMVRYTGDILAVSPPLIVDENQIGQIFEGIGKVLKEVA

SEQ ID NO: 10 - protein, 452 aa, Pseudomonas putida
MSEQNSQTLAWQTMSRDHHLAPFSDVRQLAEKGPRIITSAKGVYLWDSEGNKILDGMAGLWCVAVGYGREELAEVASQQMKQLPYYNLFFQTAHPPALELAKAIAEVAPQGMNHVFFTGSGSEGNDTVLRMVRHYWALKGQKNKKVIIGRINGYHGSTVAGAALGGMSGMHQQGGVIPDVVHIPQPYWFGEGGDMTEADFGVWAAEQLEKKILEVGVDNVAAFIAEPIQGAGGVIIPPQTYWPKIKEILARYDILFVADEVICGFGRTGEWFGTDYYDLKPDLMTIAKGLTSGYIPMGGVIVRDEVAKVISEGGDFNHGFTYSGHPVAAAVGLENLRILRDEQIIQQVHDKTAPYLQQRLRELADHPLVGEVRGLGMLGAIELVKDKATRARHEGKGVGMICRQHCFDNGLIMRAVGDTMIIAPPLVISIEEIDELVEKARKCLDLTYEAVR

SEQ ID NO: 11 - protein, 453 aa, Paracoccus denitrificans
MNQPQSWEARAETYSLYGFTDMPSVHQRGTVVVTHGEGPYIVDVHGRRYLDANSGLWNMVAGFDHKGLIEAAKAQYDRFPGYHAFFGRMSDQTVMLSEKLVEVSPFDNGRVFYTNSGSEANDTMVKMLWFLHAAEGKPQKRKILTRWNAYHGVTAVSASMTGKPYNSVFGLPLPGFIHLTCPHYWRYGEEGETEAQFVARLARELEDTITREGADTIAGFFAEPVMGAGGVIPPAKGYFQAILPILRKYDIPMISDEVICGFGRTGNTWGCLTYDFMPDAIISSKNLTAGFFPMGAVILGPDLAKRVEAAVEAIEEFPHGFTASGHPVGCAIALKAIDVVMNEGLAENVRRLAPRFEAGLKRIADRPNIGEYRGIGFMWALEAVKDKPTKTPFDANLSVSERIANTCTDLGLICRPLGQSIVLCPPFILTEAQMDEMFEKLEKALDKVFAEVA

SEQ ID NO: 12 - protein, 224 aa, Bacillus subtilis
MKIYGIYMDRPLSQEENERFMSFISPEKREKCRRFYHKEDAHRTLLGDVLVRSVISRQYQLDKSDIRFSTQEYGKPCIPDLPDAHFNISHSGRWVICAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 13 - protein, 378 aa, Bacillus subtilis
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIDLNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 14 - protein, 275 aa, Escherichia coli
MANPTVIKLQDGNVMPQLGLGVWQASNEEVITAIQKALEVGYRSIDTAAAYKNEEGVGKALKNASVNREELFITTKLWNDDHKRPREALLDSLKKLQLDYIDLYLMHWPVPAIDHYVEAWKGMIELQKEGLIKSIGVCNFQIHHLQRLIDETGVTPVINQIELHPLMQQRQLHAWNATHKIQTESWSPLAQGGKGVFDQKVIRDLADKYGKTPAQIVIRWHLDSGLVVIPKSVTPSRIAENFDVWDFRLDKDELGEIAKLDQGKRLGPDPDQFGG

SEQ ID NO: 15 - protein, 267 aa, Escherichia coli
MAIPAFGLGTFRLKDDVVISSVITALELGYRAIDTAQIYDNEAAVGQAIAESGVPRHELYITTKIWIENLSKDKLIPSLKESLQKLRTDYVDLTLIHWPSPNDEVSVEEFMQALLEAKKQGLTREIGISNFTIPLMEKAIAAVGAENIATNQIELSPYLQNRKVVAWAKQHGIHITSYMTLAYGKALKDEVIARIAAKHNATPAQVILAWAMGEGYSVIPSSTKRKNLESNLKAQNLQLDAEDKKAIAALDCNDRLVSPEGLAPEWD

SEQ ID NO: 16 - protein, 284 aa, Escherichia coli
MQQKMIQFSGDVSLPAIGQGTWYMGEDASQRKTEVAALRAGIELGLTLIDTAEMYADGGAEKVVGEALTGLREKVFLVSKVYPWNAGGQKAINACEASLRRLNTDYLDLYLLHWSGSFALEETVAAMEKLVAQGKIRRWGVSNLDYADMQELWQLPGGNQCATNQVLYHLGSRGIEYDLLPWCQQQQIPVMAYSPLAQAGRLRNGLLKNAVVNEIAHAHNISSAQVLLAWVISHQGVMAIPKAATIAHVQQNAAVLEVELSPAELAMLDKAYPAPKGKTALDMV

SEQ ID NO: 17 - protein, 349 aa, Escherichia coli
MKIKAVGAYSAKQPLEPMDITRREPGPNDVKIEIAYCGVCHSDLHQVRSEWAGTVYPCVPGHEIVGRVVAVGDQVEKYAPGDLVGVGCIVDSCKHCEECEDGLENYCDHMTGTYNSPTPDEPGHTLGGYSQQIVVHERYVLRIRHPQEQLAAVAPLLCAGITTYSPLRHWQAGPGKKVGVVGIGGLGHMGIKLAHAMGAHVVAFTTSEAKREAAKALGADEVVNSRNADEMAAHLKSFDFILNTVAAPHNLDDFTTLLKRDGTMTLVGAPATPHKSPEVFNLIMKRRAIAGSMIGGIPETQEMLDFCAEHGIVADIEMIRADQINEAYERMLRGDVKYRFVIDNRTLTD

SEQ ID NO: 18 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 19 - protein, 339 aa, Escherichia coli
MSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADF

SEQ ID NO: 20 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 21 - protein, 570 aa, Glycine max
MTTVAASLDAKEVSTSKTDQNQVCDCDPQTSHVFKSKLPDIPISNHLPLHAYCFQKLSQFSDRPCLIVGPAAKTYTYSETHLISRKIAAGLSNLGIRKGDVVMILLQNSAEFVFSFLAASMIGAVATTANPFYTAAEIFKQFTVSKTKLIITQAMYVDKLRNHDDGAKLGEDFKVVTVDDPPENCLHFSVLSEANESDAPEVDIQPDDAVAMPFSSGTTGLPKGVVLTHKSLTTSVAQQVDGENPNLYLTTEDVLLCVLPLFHIFSLNSVLLCALRAGSAVLLMQKFEIGTLLELIQRHRVSVAMVVPPLVLALAKNPMVADFDLSSIRLVLSGAAPLGKELVEALRNRVPQAVLGQGYGMTEAGPVLSMCLGFAKQPFPTKSGSCGTVVRNAELRVVDPETGRSLGYNQPGEICIRGQQIMKGYLNDEKATALTIDSEGWLHTGDVGYVDEDDEIFIVDRVKELIKYKGFQVPPAELEGLLVSHPSIADAAVVPQKDVAAGEVPVAFVVRSNGFDLTEEAVKEFIAKQVVFYKRLHKVYFVHAIPKSPSGKILRKDLRAKLETAATQTP

SEQ ID NO: 22 - protein, 157 aa, Corynebacterium glutamicum
MTTSTTPNTIISFEDAPTLTGQDLGFSQWRTVTQEMVNTFADATDDQQWIHTDPERAKDGPFGGAIAHGFLTLSMIIPFWGELLDVTGVTTKVNYGLDKVRFTSPVKVGSRIRMGAVVREISEVKGNGLHLVADGTIEIEGQERPAVVATFLTRFYA

SEQ ID NO: 23 - protein, 311 aa, Corynebacterium glutamicum
MSLNGKVAIVTGSGAGLGRSFAQELARQGASVIVNDVNQAAADETVAAITEAGGKAAAVIAPVGPSESAALLVREAVDKFGSLDILVTNAGILRDRSLLKMTDDDFDAVINVHLKGTFTCVRETFGYFKENGIAGRIVTIGSPTGQRGNFGQSNYAAAKAGIVGMVRTWALEMKRAGVTINAIIPEAATDMTKTVPYFQKAVEADERGEAMPAFFRETLGFGTPQDVAGLVAFLSSDEAANISGQAIGAGGDRMQVWKHPEPAVTEFNPGGWTYEALQERGKNIIEDNLQSVGVVFPELPAELQPQIPVKA

SEQ ID NO: 24 - protein, 314 aa, Corynebacterium glutamicum
MSNNVVKYECAVDADNIVAVDMHVHLEVDSCGHESMPADIMEASSKYFKAAERTPSADAIADIYREHKMAAVVFTIDARTQMGHLPNSIDDLVASCARNNDVLIPFGSVDPRTGEDALVEARRQVEELGVRGFKFHPSVQGFDPSAPEFYPLWELLESFGLPCVFHTGQNGMGAGLPGGRGIKLRFSNPLLLDDIAADFPNLTIIMAHPSVPWQDEANSIATHKANVFIDLSGWSPKYFPESLVRQSNNVLSKKVLFGTDFPLITPEKWLAAFANLPLKDEVRPGILKDNAVKVLGLAVNTERGSQAEKAVQHA

SEQ ID NO: 25 - protein, 456 aa, Pseudomonas aeruginosa
MNSQITNAKTREWQALSRDHHLPPFTDYKQLNEKGARIITKAEGVYIWDSEGNKILDAMAGLWCVNVGYGREELVQAATRQMRELPFYNLFFQTAHPPVVELAKAIADVAPEGMNHVFFTGSGSEANDTVLRMVRHYWATKGQPQKKVVIGRWNGYHGSTVAGVSLGGMKALHEQGDFPIPGIVHIAQPYWYGEGGDMSPDEFGVWAAEQLEKKILEVGEENVAAFIAEPIQGAGGVIVPPDTYWPKIREILAKYDILFIADEVICGFGRTGEWFGSQYYGNAPDLMPIAKGLTSGYIPMGGVVVRDEIVEVLNQGGEFYHGFTYSGHPVAAAVALENIRILREEKIIEKVKAETAPYLQKRWQELADHPLVGEARGVGMVAALELVKNKKTRERFTDKGVGMLCREHCFRNGLIMRAVGDTMIISPPLVIDPSQIDELITLARKCLDQTAAAVLA

SEQ ID NO: 26 - DNA, 10496 bp, synthetic construct
  source 1..10496: mol_type = other DNA
gaattcaaaagatctgagtcttgtaactcccgtattcattttgaacatcgggacaatccaatatatgtacggttaagtggtatccgcagtcggatacggcaaaggaggatttcaaatgcagaaacagcggactaccagccagtggcgcgaacttgatgccgcccatcatctacatccgtttaccgacaccgcctccctgaatcaagcgggagcgagggtgatgacccgcggcgaaggcgtatacttatgggattctgaagggaacaaaattatcgatggcatggcgggcctgtggtgcgtgaatgtgggttacggccgcaaggacttcgcggaggcggcccgccgccagatggaagaattgccgttttacaacacgtttttcaagaccacccatcctgcagtggtcgagctcagctccttactggcggaagtcacccctgcaggttttgatcgtgttttctatacaaactccggcagtgaatccgtggataccatgatccgcatggtacgccgctattgggatgtgcaagggaaaccggaaaagaaaacgctgattggccgatggaacggctaccatggcagcaccattggtggtgcgtcattgggtgggatgaaatatatgcacgagcagggcgatctgccaattccgggaatggcccacatagagcagccttggtggtataaacatggtaaagacatgaccccggacgagttcggcgtggtggccgcccgttggctggaagagaaaatcctcgaaatcggagcggacaaggtggcggcctttgttggtgagcccattcagggcgctggaggtgttattgtcccgcccgccacttattggccggaaattgaacgtatttgccgaaaatatgatgtcttactggtggccgatgaagtgatatgcggcttcggccgtacaggcgaatggttcggtcatcaacatttcggttttcagccggatttgttcacggcggccaaaggtttgtcctcaggctatctgccgataggagcagtatttgttggtaaacgcgtggccgaagggctgattgcgggcggcgatttcaaccatggttttacctacagcggtcatccggtctgcgccgcagtggcgcatgcgaacgttgctgcacttcgcgacgagggcattgttcagcgtgtgaaggatgatattggcccgtatatgcaaaaacgatggcgcgaaacatttagccgttttgaacacgttgatgacgtacgcggcgttggcatggtgcaggcctttacccttgtgaagaacaaagcgaaacgcgagctgtttccggattttggtgaaattggcacgttgtgtcgtgatattttctttcgtaacaatctgataatgcgtgcctgcggggatcacatagtatcggcaccgccgctggtgatgacgcgcgccgaggtcgatgagatgttggccgtggccgagcgctgtcttgaagaatttgaacaaaccttgaaagcgcgtggactagcataatctatatgtgacggtaataggcgtcacggtcagtcaatcggttctcatgtagcagatccgagaaagcggattcaagttagtgaggtcgaatgataattggcgttccaaaggaaattaaaaataacgagaaccgcgtcgcactgacccccggtggcgtgtcacagctgattagcaatggccacagggtgttagttgagacaggagctggcctgggctccggctttgaaaatgaagcgtatgaaagtgcgggggcggagattattgccgatccgaaacaagtgtgggacgcggagatggttatgaaggtaaaggagccgctgccggaggagtatgtctacttccgtaagggtcttgttttgttcacttaccttcatctggcagcggagccggaactggcccaggcacttaaagacaaaggcgtgaccgccatagcatacgaaacggtgtcggaaggccgtactctgccgttattaacgcccatgtctgaggtggcaggacgcatggcggctcaaattggcgcgcagttcctggaaaaacccaagggtggcaaaggcattttactggccggcgtgccgggcgtttcgcgtggcaaagtgaccattataggtgggggcgtggtgggcaccaatgcggccaagatggcggttggtctgggcgcagacgtaaccattattgaccttaatgcggatcggctgagacagctggatgatatttttgggcatcagattaaaaccctgattagcaacccggtgaacatcgccgacgctgtggcggaggccgatcttttgatttgcgccgtcctgataccaggagcgaaagccccgaccttggtgacggaagaaatggtgaaacagatgaagccgggctccgtaattgttgacgtggcaatcgaccaaggcggaattgttgaaaccgttgatcatatcacgacccatgatcaacccacgtacgagaaacatggcgtcgtccattacgctgtcgctaatatgccgggcgcggtgcctcgcacttcgacaattgccctgactaatgtcaccgttccctacgcgctgcagatcgctaacaagggcgcggtgaaagccctggctgacaacacggccttaagggcgggcttaaatacggcgaacggccatgttacctacgaagcagtggcccgcgaccttggatatgaatatgtcccggccgaaaaagccctgcaggatgagagtagcgtggctggagcctaacgtctgacggcgataacggatgtttccatggacgctttaatctgggatggtttaataagctaagtgggatgaaaggaggtaaggaatggcggtggattctccggatgaacgccttcagcgccgcattgcgcagctatttgcggaagacgagcaggtgaaagcggcacgtccgctggaggcggtgagcgcagcagtcagcgcacccggcatgaggctggcacaaatcgctgcgaccgtgatggcaggctatgccgaccgtccggccgcaggccagcgcgcatttgagctaaacaccgatgatgccacgggccggacgagcttacgcctcctgccccgttttgaaactattacctatcgcgaactgtggcagcgagtgggcgaagttgcggcagcgtggcatcatgatcccgaaaatccactgcgtgccggcgattttgtggccctgctgggttttacatcgatagattacgcaacactggatctggcggacattcatttaggggccgtgacagtaccgctgcaagcgtctgcggcggttagccaactgattgcaattctaaccgaaaccagccctcggctccttgcgtcaaccccggaacatctggatgcggcggtggaatgtttattggccggtacaacaccggaacgcctggtggtttttgactatcaccccgaagacgatgatcagcgcgcggcctttgaaagtgcgcgccgacgcttggccgacgcaggctcgctggtgattgttgagacgctcgatgctgtgcgcgcgcgcggacgtgatctaccggcagcacccctgtttgtcccggacacagacgacgatccactggccctgctgatttatacgagcgggagtaccggcacccctaaaggcgcgatgtacaccaatcgcctggcggcaaccatgtggcaaggtaacagcatgcttcagggcaatagccaacgcgttggtatcaaccttaattatatgccaatgtcacatattgcaggacgcatctcgttgtttggagtacttgcacgcggtggcaccgcatactttgcggcgaaatccgatatgtccacgttattcgaggatattggcttggtacgcccaacggagattttctttgtgccacgcgtatgtgatatggtgttccagcgctatcaaagcgaactggatcgccgctctgtagcaggtgcggatttagatacactggaccgagaagtgaaggcagacctgcgtcagaactacctgggtggtcgctttctcgttgcggtcgtgggtagcgcacccttggcagctgaaatgaaaacgttcatggaatcagtactggatttgccattacatgacggctatggctcaaccgaagccggcgcaagcgtactgcttgataaccagattcagcgccctccggttctggactacaagctggtggatgttccagaactcggttattttcgtactgataggccgcacccgcgcggtgaactcttattaaaggcagaaactactattccgggctattataaacgtccggaagtgaccgcggaaatctttgacgaggatggtttttacaaaactggcgatatcgtagcggagttagagcacgaccgcctggtatacgtggaccgccgaaataacgtgctcaagctgtcgcagggagagttcgtgactgtagcccacctagaagccgtgtttgcatccagcccgttgatccgacagatttttatctacggtagttctgagcggagctacctgttggcagtaattgtgccgaccgatgacgcgctgcggggacgcgataccgccaccctgaagtccgctttggcggaatcgattcaacgcatcgcgaaagatgcaaatctgcagccgtatgaaataccgcgcgatttcctgatcgaaacagaaccgtttacgatcgcaaatggtctgctttcgggcattgccaaacttttacgtcctaacctgaaagagcgctacggcgcccaactggagcagatgtacaccgatctagccaccgggcaggcagatgaactcttagcattacgtcgcgaagctgcggacctcccggtgctcgaaactgttagtcgcgcagcaaaggcgatgctgggcgtcgcgagtgcagatatgcgccccgatgcccattttactgacctgggtggcgattccctgtctgcgctgtccttttccaatctgctacacgaaattttcggtgtagaagtgcctgtaggtgtagtcgtcagcccggcaaacgaacttcgcgatctggcgaattacattgaggccgaaaggaacagtggcgcgaaacgccccacttttactagcgtacacggcggtggctccgaaattcgcgcggcagacctcactctggacaaatttattgatgcgcgcaccctggccgcggcagattcgattcctcacgcgcctgttccggcccagacggtgctgctgaccggcgcaaacgggtacttaggacgctttctttgtcttgaatggctggaacggctcgataaaacgggcggtacgctgatatgtgttgtgcgtggaagcgacgcggcggcggcgcgcaagcgcctagattcagcgttcgatagcggagatcccggcttgctggaacattatcaacagctggccgcccgcaccctggaagttctagcgggggatattggtgatcctaatctgggcctcgatgatgccacatggcagcgtctggcagaaacagttgacctgatcgtgcatccggcagcgttggtgaaccacgtgctgccgtatacgcaattgttcggtccgaatgtcgtcggcaccgcagaaatcgtacgtttagctatcactgcccgacgtaagcctgtgacgtacttgtcgactgtaggtgtggcggatcaggtggaccccgccgaatatcaggaagattcggatgtacgcgagatgagcgccgtgcgcgtagtgcgcgaaagctatgccaatgggtacggtaactcgaaatgggcgggtgaagtgctgttacgcgaagcacatgatctgtgcggtttgccggtcgcagtatttcggagcgatatgatcttagcgcactcgcgctacgcgggccagctgaatgtgcaggacgtgtttacccgtctgatactctcactggtggcgactggcatcgcaccgtatagtttctatcggacagatgcggatggtaatcgacagcgtgcacattatgatggccttcccgcggattttacggccgccgcgataacggcgctcggaatccaggctacggaaggctttcgcacttatgacgtcttgaacccctatgacgatggtatttctctggatgagtttgttgactggctcgtagaaagcggtcacccgatccagcgcattacagattactctgattggtttcatcgttttgaaaccgcgattcgtgcgttaccagaaaaacagcgccaagcgtcagtcctaccgctcttggatgcgtaccggaatccgtgtccggccgttcgcggtgcaatcttaccggcgaaagaatttcaagctgccgttcaaaccgctaaaattggcccggaacaggacattccgcaccttagtgcccctctgattgataaatatgtgagcgatctggaactgctgcaactgctctaacctgtagaaataattttgtttaactttaataaggagatataccatgggcaagatttacggaatttatatggaccgcccgctttcacaggaagaaaatgaacggttcatgactttcatatcacctgaaaaacgggagaaatgccggagattttatcataaagaagatgctcaccgcaccctgctgggagatgtgctcgttcgctcagtcataagcaggcagtatcagttggacaaatccgatatccgctttagcacgcaggaatacgggaagccgtgcatccctgatcttcccgacgctcatttcaacatttctcactccggccgctgggtcattggtgcgtttgattcacagccgatcggcatagatatcgaaaaaacgaaaccgatcagccttgagatcgccaagcgcttcttttcaaaaacagagtacagcgaccttttagcaaaagacaaggacgagcagacagactatttttatcatctatggtcaatgaaagaaagctttatcaaacaggaaggcaaaggcttatcgcttccgcttgattccttttcagtgcgcctgcatcaggacggacaagtatccattgagcttccggacagccattccccatgctatatcaaaacgtatgaggtcgatcccggctacaaaatggctgtatgcgccgcacaccctgatttccccgaggatatcacaatggtctcgtacgaagagcttttataacaaataaaacgaaaggctcagtcgaaagactgggcctttcgttttatctgttgtttgtcggtgaacgctctctactagagtcacactggctcaccttcgggtgggcctttctgcgtttatacctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcatgatcccctgcgccatcagatccttggcggcaagaaagccatccagtttactttgcagggcttcccaaccttaccagagggcgccccagctggcaattccgacgtcgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttca

SEQ ID NO: 27 - protein, 299 aa, Escherichia coli
MKREEICRLLADKVNKLKNKENSLSELLPDVRLLYGETPFARTPVMYEPGIIILFSGHKIGYINERVFRYDANEYLLLTVPLPFECETYATSEVPLAGLRLNVDILQLQELLMDIGEDEHFQPSMAASGINSATLSEEILCAAERLLDVMERPLDARILGKQIIREILYYVLTGPCGGALLALVSRQTHFSLISRVLKRIENKYTENLSVEQLAAEANMSVSAFHHNFKSVTSTSPLQYLKNYRLHKARMMIIHDGMKASAAAMRVGYESASQFSREFKRYFGVTPGEDAARMRAMQGN

SEQ ID NO: 28 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 29 - protein, 6 aa, synthetic construct
HHHHHH

(19) United States
(12) Patent Application Publication
(10) Pub. No.: US 2026/0265793 A1
(43) Pub. Date: Sep. 10, 2026

(54) AMINE SYNTHESIS

(71) Applicant: C3 BIOTECHNOLOGIES LIMITED, Lancaster (GB)

(72) Inventor: Jonathan WILKES, Lancaster (GB)

(21) Appl. No.: 19/480,005

(22) PCT Filed: May 1, 2024
(86) PCT No.: PCT/EP2024/061993

§ 371 Date: Oct. 29, 2025

(30) Foreign Application Priority Data
May 2, 2023 (GB) 2306433.0

(51) Int. Cl. C12P 13/00; C12N 1/20; C12N 9/02; C12N 9/06; C12N 9/10; C12N 9/12; C12N 15/70; C12P 17/18; C12R 1/19

(52) U.S. Cl. CPC C12P 13/001; C12N 1/20; C12N 9/0008; C12N 9/0016; C12N 9/1096; C12N 9/1288; C12N 15/70; C12P 17/182; C12R 2001/19; C12Y 102/0103; C12Y 104/01001; C12Y 206/01018; C12Y 207/08007

(57) ABSTRACT

Disclosed herein are methods for the production of amines, nucleic acids for use in methods for the production of amines, enzymes for use in methods for the production of amines, methods of producing a compound utilising an amine produced by a method disclosed herein, and organisms for the production of amines.

This application claims priority from GB2306433.0 filed 2 May 2023, the contents and elements of which are herein incorporated by reference for all purposes.

### FIELD OF THE INVENTION

The present invention relates to the fields of molecular biology and biotechnology and particularly, although not exclusively, to methods of producing amines.

### BACKGROUND

The synthesis of many commercially-important compounds is currently reliant on the availability of amine precursors derived from non-sustainable fossil fuels and highly toxic metal catalysts. This process results in the generation of hazardous waste which requires costly disposal. As a result, the widespread application of such compounds is limited due to the prohibitive costs of current synthesis routes.

Benzylamine is a precursor to the synthesis of many commercial-important compounds including motion-sickness treatments, anticonvulsants and the high-energy propellant hexanitrohexaazaisowurtzitane (CL-20). Biosynthetic routes for the synthesis of benzylamine have recently been explored due to the disadvantages associated with current synthesis routes.

Zhou et al. 2018 discloses biosynthetic routes to benzylamine under biotransformation conditions from L-phenylalanine and under fed-batch fermentation conditions from glucose (obtaining a titre of 0.46 g/L from glucose). Pandey et al. 2021 discloses a shorter biosynthetic route to benzylamine from phenylpyruvate but this did not improve titres (0.024 g/L). A much greater yield will be required to make these processes commercially viable at an industrial scale.

The present inventors have identified a novel biosynthetic route to benzylamine from benzoic acid utilising carboxylic acid reductase (CAR) and transaminase (TA) enzymes (FIG. 1) which has produced a benzylamine titre of 3.4 g/L.

### SUMMARY OF THE INVENTION

In one aspect, the present disclosure provides a method of producing an amine, wherein the method comprises: (a) the conversion of a carboxylic acid substrate into an aldehyde catalysed by a carboxylic acid reductase (CAR), wherein the carboxylic acid substate is a carboxylic acid, a conjugate base of a carboxylic acid, a salt of a carboxylic acid or an ester of a carboxylic acid; and (b) the conversion of the aldehyde into an amine catalysed by a transaminase (TA).

The present disclosure also provides a method of producing or synthesising an amine, wherein the method comprises providing a microorganism expressing a carboxylic acid reductase (CAR) and a transaminase (TA), and providing a carboxylic acid substrate, wherein the carboxylic acid substrate is contacted with the CAR and is converted to an aldehyde through CAR activity, and the aldehyde is contacted with the TA and is converted to an amine through TA activity.

In some embodiments, the carboxylic acid substrate is a carboxylic acid. In some embodiments, the carboxylic acid substrate is a conjugate base of a carboxylic acid (also known as a carboxylate). In some embodiments, the carboxylic acid substrate is a carboxylic acid or a conjugate base of a carboxylic acid. In some embodiments, the carboxylic acid substrate is selected from a carboxylic acid, a conjugate base of a carboxylic acid, a salt of a carboxylic acid (also known as a carboxylate salt) or an ester of a carboxylic acid (also known as a carboxylate ester).

The salt of a carboxylic acid may be, for example, a sodium salt, a potassium salt or a zinc salt.

In some embodiments, the carboxylic acid is an alkanoic acid (i.e. a straight-chain saturated carboxylic acid) (e.g. methanoic (formic) acid, ethanoic (acetic) acid, propanoic acid or butanoic acid), an unsaturated monocarboxylic acid (e.g. acrylic acid), a fatty acid (e.g. docosahexaenoic acid), an amino acid (e.g. alanine), a keto acid (e.g. acetoacetic acid), an aromatic carboxylic acid (e.g. benzoic acid), a dicarboxylic acid (e.g. adipic acid), a tricarboxylic acid (e.g. citric acid), an alpha hydroxy acid (e.g. glyceric acid), a beta hydroxy acid, an omega hydroxy acid or a divinylether fatty acid. In some embodiments, the carboxylic acid is benzoic acid.

In some embodiments, the conjugate base of a carboxylic acid is the conjugate base of any of the carboxylic acids disclosed herein. In some embodiments, the conjugate base of a carboxylic acid is benzoate.

In some embodiments, the salt of benzoic acid may be sodium benzoate, potassium benzoate or zinc benzoate.

In some embodiments, a carboxylic acid substate may be provided and then be converted into a different carboxylic acid substrate before being converted into an aldehyde.

References in the present disclosure to a particular type of carboxylic acid substrate are also references to the other types of carboxylic acid substrate disclosed herein. For example, a reference to a carboxylic acid is also a reference to a conjugate base of a carboxylic acid, a salt of a carboxylic acid and an ester of a carboxylic acid. In addition, a reference to benzoic acid is also a reference to the conjugate base of benzoic acid, a salt of benzoic acid and an ester of benzoic acid.

In some embodiments, the aldehyde is the aldehyde form of any of the carboxylic acid substrates disclosed herein. In some embodiments, the aldehyde is benzaldehyde.

In some embodiments, the amine is the amine form of any of the aldehydes disclosed herein. In some embodiments, the amine is benzylamine.

In some embodiments, the carboxylic acid substrate is provided exogenously to the microorganism. In some embodiments, the carboxylic acid substrate is an exogenous carboxylic acid substrate.

In some embodiments, the carboxylic acid is provided exogenously to the microorganism. In some embodiments, the carboxylic acid is exogenous carboxylic acid. In some embodiments, the benzoic acid is provided exogenously to the microorganism. In some embodiments, the benzoic acid is exogenous benzoic acid.

In some embodiments, the conjugate base of a carboxylic acid is provided exogenously to the microorganism. In some embodiments, the conjugate base of a carboxylic acid is an exogenous conjugate base of a carboxylic acid. In some embodiments, the benzoate is provided exogenously to the microorganism. In some embodiments, the benzoate is exogenous benzoate.

In some embodiments, the carboxylic acid is provided in an isolated or purified form. In some embodiments, the carboxylic acid is provided as a mixture. In some embodiments, the carboxylic acid is provided at a purity of, or at a purity of at least 50%. In some embodiments, the carboxylic acid is provided at a purity of, or at a purity of at least 55%, 60%, 65%, 70%, 75%, 80%, 85%, 90%, 95%, 96%, 97%, 98%, 99%, or 100%.

In some embodiments, carboxylic acid is provided at a concentration of less than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or less than 200 mM. In some embodiments, benzoic acid is provided at a concentration of less than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or less than 200 mM.

In some embodiments, carboxylic acid is provided at a concentration of more than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or more than 200 mM. In some embodiments, benzoic acid is provided at a concentration of more than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or more than 200 mM.

In some embodiments, carboxylic acid is provided at a concentration of 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or 200 mM. In some embodiments, benzoic acid is provided at a concentration of 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or 200 mM.

In some embodiments, carboxylic acid is provided at a concentration between 10 mM and 20 mM, 10 mM and 30 mM, 10 mM and 40 mM, 10 mM and 50 mM, 10 mM and 60 mM, 10 mM and 70 mM, 10 mM and 80 mM, 10 mM and 90 mM, 10 mM and 100 mM, 10 mM and 110 mM, 10 mM and 120 mM, 10 mM and 130 mM, 10 mM and 140 mM, 10 mM and 150 mM, 10 mM and 160 mM, 10 mM and 170 mM, 10 mM and 180 mM, 10 mM and 190 mM, or 10 mM and 200 mM.

In some embodiments, carboxylic acid is provided at a concentration between 20 mM and 30 mM, 20 mM and 40 mM, 20 mM and 50 mM, 20 mM and 60 mM, 20 mM and 70 mM, 20 mM and 80 mM, 20 mM and 90 mM, 20 mM and 100 mM, 20 mM and 110 mM, 20 mM and 120 mM, 20 mM and 130 mM, 20 mM and 140 mM, 20 mM and 150 mM, 20 mM and 160 mM, 20 mM and 170 mM, 20 mM and 180 mM, 20 mM and 190 mM, or 20 mM and 200 mM.

In some embodiments, carboxylic acid is provided at a concentration between 50 mM and 60 mM, 50 mM and 70 mM, 50 mM and 80 mM, 50 mM and 90 mM, 50 mM and 100 mM, 50 mM and 110 mM, 50 mM and 120 mM, 50 mM and 130 mM, 50 mM and 140 mM, 50 mM and 150 mM, 50 mM and 160 mM, 50 mM and 170 mM, 50 mM and 180 mM, 50 mM and 190 mM, or 50 mM and 200 mM.

In some embodiments, carboxylic acid is provided at a concentration between 100 mM and 110 mM, 100 mM and 120 mM, 100 mM and 130 mM, 100 mM and 140 mM, 100 mM and 150 mM, 100 mM and 160 mM, 100 mM and 170 mM, 100 mM and 180 mM, 100 mM and 190 mM, or 100 mM and 200 mM.

The inventors have investigated the effect of increasing the number of carboxylic acid doses. In some cases, increasing the dose can increase the yield of amine (e.g. benzylamine). For example, it is shown in FIG. 10 that benzylamine yield is considerably higher with three 50 mM doses (feeds) compared to a single 50 mM carboxylic acid dose.

In some embodiments, one dose of carboxylic acid is provided. In some embodiments, multiple doses of carboxylic acid are provided. In some embodiments, 2, 3, 4, 5, 6, 7, 8, 9, or 10 doses of carboxylic acid are provided. In some embodiments, each dose of carboxylic acid is provided at a concentration of less than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or less than 200 mM. In some embodiments, each dose of carboxylic acid is provided at a concentration of more than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or more than 200 mM. In some embodiments, each dose of carboxylic acid is provided at a concentration of 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or 200 mM.

In some embodiments, one dose of benzoic acid is provided. In some embodiments, multiple doses of benzoic acid are provided. In some embodiments, 2, 3, 4, 5, 6, 7, 8, 9, or 10 doses of carboxylic acid are provided. In some embodiments, each dose of benzoic acid is provided at a concentration of less than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or less than 200 mM. In some embodiments, each dose of benzoic acid is provided at a concentration of more than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or more than 200 mM. In some embodiments, each dose of benzoic acid is provided at a concentration of 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or 200 mM.

In some embodiments, the method comprises two stages: (i) a growth stage, and (ii) a conversion stage.

In some embodiments, the microorganism is cultured in a growth medium in the growth stage.

In some embodiments, a substrate is provided in the conversion stage. In some embodiments, carboxylic acid is provided in the conversion stage. In some embodiments, benzoic acid is provided in the conversion stage.

In some embodiments, the method comprises microbial fermentation. In some embodiments, the method comprises whole cell biocatalysis. In some embodiments, the method comprises biotransformation.

In some embodiments, the growth stage comprises microbial fermentation. In some embodiments, the conversion stage comprises biotransformation.

In some embodiments, the microorganism is separated from the media. In some embodiments, the microorganism is resuspended. In some embodiments, the microorganism is separated from the growth medium and resuspended in a buffer. In some embodiments, the microorganism is separated from the microbial growth medium and resuspended in a buffer for the conversion stage.

In some embodiments, the microorganism is resuspended at a cell density wherein an optical density (OD₆₀₀ₙₘ) measurement is above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or above 100.

In some embodiments, the microorganism is resuspended at a cell density wherein an OD₆₀₀ₙₘ measurement is below 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or below 100.

In some embodiments, the microorganism is resuspended at a cell density wherein an OD₆₀₀ₙₘ measurement is between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 1 and 16, 1 and 17, 1 and 18, 1 and 19, 1 and 20, 1 and 21, 1 and 22, 1 and 23, 1 and 24, 1 and 25, 1 and 26, 1 and 27, 1 and 28, 1 and 29, 1 and 30, 1 and 31, 1 and 32, 1 and 33, 1 and 34, 1 and 35, 1 and 36, 1 and 37, 1 and 38, 1 and 39, 1 and 40, 1 and 41, 1 and 42, 1 and 43, 1 and 44, 1 and 45, 1 and 46, 1 and 47, 1 and 48, 1 and 49, 1 and 50, 1 and 51, 1 and 52, 1 and 53, 1 and 54, 1 and 55, 1 and 56, 1 and 57, 1 and 58, 1 and 59, 1 and 60, 1 and 61, 1 and 62, 1 and 63, 1 and 64, 1 and 65, 1 and 66, 1 and 67, 1 and 68, 1 and 69, 1 and 70, 1 and 71, 1 and 72, 1 and 73, 1 and 74, 1 and 75, 1 and 76, 1 and 77, 1 and 78, 1 and 79, 1 and 80, 1 and 81, 1 and 82, 1 and 83, 1 and 84, 1 and 85, 1 and 86, 1 and 87, 1 and 88, 1 and 89, 1 and 90, 1 and 91, 1 and 92, 1 and 93, 1 and 94, 1 and 95, 1 and 96, 1 and 97, 1 and 98, 1 and 99, or between 1 and 100. In some embodiments, the microorganism is resuspended at a cell density wherein an OD₆₀₀ₙₘ measurement is between 10 and 20, 15 and 25, 20 and 30, 25 and 35, 30 and 40, 35 and 45, 40 and 50, 45 and 55, 50 and 60, 55 and 65, 60 and 70, 65 and 75, 70 and 80, 75 and 85, 80 and 90, 85 and 95, or between 90 and 100.

In some embodiments, the microorganism is fresh. In some embodiments, the microorganism is hydrated. In some embodiments, the microorganism is non-lyophilised. In some embodiments, the microorganism is viable. In some embodiments, the microorganism is not lyophilised before the conversion stage.

In some embodiments, the method comprises three stages: (i) a carboxylic acid production stage, (ii) a growth stage, and (iii) a conversion stage.

In some embodiments, the carboxylic acid production stage and the growth stage are performed separately. In some embodiments, the carboxylic acid production stage and the growth stage are performed separately and simultaneously. In some embodiments, the carboxylic acid production stage is performed before the growth stage.

In some embodiments, the microorganism is a bacterium, a fungus, a cyanobacterium, or an alga. In some embodiments, the microorganism is a bacterium. In some embodiments, the microorganism is a gram-positive bacterium. In some embodiments, the microorganism is a gram-negative bacterium.

In some embodiments, the bacterium is selected from the genus Escherichia, Listeria, Clostridium, Staphylococcus, Streptococcus, Pseudomonas, Helicobacter, Neisseria, Legionella, Halomonas, Klebsiella or Yersinia. In some embodiments, the bacterium is Escherichia coli.

In some embodiments, amine production occurs within the cell of the microorganism. In some embodiments, amine biosynthesis occurs within the cell of the microorganism. In some embodiments, conversion of carboxylic acid to amine occurs within the cell of the microorganism. In some embodiments, conversion of carboxylic acid to aldehyde occurs within the cell of the microorganism. In some embodiments, conversion of aldehyde to amine occurs within the cell of the microorganism.

In some embodiments, amine production occurs within the buffer or cell lysate. In some embodiments, amine biosynthesis occurs within the buffer or cell lysate. In some embodiments, conversion of carboxylic acid to amine occurs within the buffer or cell lysate. In some embodiments, conversion of carboxylic acid to aldehyde occurs within the buffer or cell lysate. In some embodiments, conversion of aldehyde to amine occurs within the buffer or cell lysate.

In some embodiments, the carboxylic acid or conjugate base is contacted with the CAR within the cell of the microorganism. In some embodiments, the aldehyde is contacted with the TA within the cell of the microorganism.

In some embodiments, the carboxylic acid or conjugate base is contacted with the CAR within the buffer or cell lysate. In some embodiments, the aldehyde is contacted with the TA within the buffer or cell lysate.

In some embodiments, the microorganism is genetically modified to express a heterologous CAR and/or a heterologous TA.

The CAR is functional. The CAR comprises carboxylic acid reductase activity.

In some embodiments, the CAR comprises an amino acid sequence with at least 40% sequence identity to SEQ ID NO:1, 2, 3, 4, 5, or 6. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NOs: 1, 2, 3, 4, 5, or 6.

In some embodiments, the CAR is a wild type or mutant CAR derived from Nocardia iowensis.

In some embodiments, the TA is an omega-TA (ω-TA).

The TA is functional. The TA comprises transaminase activity.

In some embodiments, the ω-TA comprises an amino acid sequence with at least 40% sequence identity to SEQ ID NO:7, 8, 9, 10, 11 or 25. In some embodiments, the ω-TA comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NOs: 7, 8, 9, 10, 11 or 25.

In some embodiments, the ω-TA is a wild type or mutant ω-TA derived from Chromobacterium violaceum or from Vibrio fluvialis.

Reduction is a chemical reaction in which a substrate gains electrons and/or the substate's oxidation state is decreased. In some embodiments, the microorganism has been modified to inhibit the reduction of aldehydes, preferably aromatic aldehydes, more preferably benzaldehyde. In some embodiments, the microorganism has been modified to inhibit the reduction of aldehydes into their corresponding alcohols, preferably the reduction of aromatic aldehydes into aromatic alcohols, more preferably the reduction of benzaldehyde into benzyl alcohol.

In some embodiments, the organism has been modified to reduce or eliminate the activity or expression of one or more enzymes which catalyse the reduction of aldehydes, preferably aldehyde reductases, aldo-keto reductases (AKRs) and/or alcohol dehydrogenases (ADHs). In some embodiments, the organism has been modified to reduce or eliminate the activity or expression of an aldehyde reductase, AKR or ADH enzyme which comprises an amino acid sequence with at least 40% sequence identity to one or more of SEQ ID NO:14, 15, 16, 17, 18, or 19. In some embodiments, the aldehyde reductase, AKR or ADH comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NOs: 14, 15, 16, 17, 18, or 19.

In some embodiments, the carboxylic acid is produced industrially. In some embodiments, the carboxylic acid is produced industrially by chemical oxidation. In some embodiments, the carboxylic acid is produced biologically.

In some embodiments, the carboxylic acid is produced by an organism capable of producing benzoic acid from phenylalanine. In some embodiments, the carboxylic acid is produced by an organism capable of producing benzoic acid from phenylalanine in the way demonstrated in FIG. 1. In some embodiments, the organism is a microorganism.

In some embodiments, the organism capable of producing benzoic acid from phenylalanine is capable of converting phenylalanine to cinnamate, converting cinnamate to cinnamoyl-CoA, converting cinnamoyl-CoA to 3-hydroxy-3-phenylpropionyl-CoA (3-HPP CoA), converting 3-HPP CoA to 3-keto-3-phenylpropionyl-CoA (3-KPP CoA), and converting 3-KPP CoA to benzoic acid.

In some embodiments, the organism is capable of producing benzoic acid. In some embodiments, the organism is capable of producing benzoic acid from phenylalanine. In some embodiments, the organism is capable of producing benzoic acid from glucose. In some embodiments, the organism is a microorganism.

In some embodiments, the organism capable of producing benzoic acid comprises the enzymes capable of producing benzoic acid from phenylalanine. In some embodiments, the organism capable of producing benzoic acid from phenylalanine comprises a phenylalanine ammonia lyase, a coumarate-CoA-ligase, and phenylpropanoid degradation pathway enzymes. In some embodiments, the organism capable of producing benzoic acid from phenylalanine comprises the enzymes shown in Pathway IIIA of FIG. 1.

In some embodiments, the organism capable of producing benzoic acid comprises an enzyme comprising an amino acid sequence with at least 40% sequence identity to the amino acid sequence of Arabidopsis thaliana phenylalanine ammonia-lyase 2 (PAL2; AtPAL2) (UniProt: P45724). In some embodiments, the enzyme comprises an amino acid sequence with at least 40% sequence identity to SEQ ID NO:20. In some embodiments, the enzyme comprises, or consists, of an amino acid sequence having at least 45%, 50%, 55%, 60%, 65%, 70%, 75%, 80%, 85%, 90%, 95%, 99%, or 100% sequence identity to SEQ ID NO: 20.

In some embodiments, the organism capable of producing benzoic acid comprises an enzyme comprising an amino acid sequence with at least 40% sequence identity to the amino acid sequence of Glycine max 4-coumarate: CoA ligase (4CL; Gm4CL) (UniProt: Q8S5C2). In some embodiments, the enzyme comprises an amino acid sequence with at least 40% sequence identity to SEQ ID NO:21. In some embodiments, the Gm4CL comprises, or consists, of an amino acid sequence having at least 45%, 50%, 55%, 60%, 65%, 70%, 75%, 80%, 85%, 90%, 95%, 99%, or 100% sequence identity to SEQ ID NO:21.

In some embodiments, the organism capable of producing benzoic acid comprises one or more Corynebacterium glutamicum phd enzymes (phdB, phdC and/or phdE), variants of Corynebacterium glutamicum phd enzymes, or enzymes which are homologous to Corynebacterium glutamicum phd enzymes. In some embodiments, the organism capable of producing benzoic acid comprises one or more of Corynebacterium glutamicum phdB (3-hydroxyacyl-CoA dehydrogenase), Corynebacterium glutamicum phdC (3-oxoacyl-CoA ketohydrolase) (acetyl-CoA forming), or Corynebacterium glutamicum phdE (enoyl-CoA hydratase).

In some embodiments, the organism capable of producing benzoic acid comprises an enzyme comprising an amino acid sequence with at least 40% sequence identity to the amino acid sequence of phdE from Corynebacterium glutamicum (GENBANK: AGN20978.1). In some embodiments, the enzyme comprises an amino acid sequence with at least 40% sequence identity to SEQ ID NO:22. In some embodiments, the phdE comprises, or consists, of an amino acid sequence having at least 45%, 50%, 55%, 60%, 65%, 70%, 75%, 80%, 85%, 90%, 95%, 99%, or 100% sequence identity to SEQ ID NO:22.

In some embodiments, the organism capable of producing benzoic acid comprises an enzyme comprising an amino acid sequence with at least 40% sequence identity to the amino acid sequence of phdB from Corynebacterium glutamicum (GENBANK: UniProt: AGN20975.1). In some embodiments, the enzyme comprises an amino acid sequence with at least 40% sequence identity to SEQ ID NO:23. In some embodiments, the phdB comprises, or consists, of an amino acid sequence having at least 45%, 50%, 55%, 60%, 65%, 70%, 75%, 80%, 85%, 90%, 95%, 99%, or 100% sequence identity to SEQ ID NO:23.

In some embodiments, the organism capable of producing benzoic acid from phenylalanine comprises an enzyme which comprises an amino acid sequence with at least 40% sequence identity to the amino acid sequence of phdC from Corynebacterium glutamicum (GENBANK: UniProt: AGN20976.1). In some embodiments, the enzyme comprises an amino acid sequence with at least 40% sequence identity to SEQ ID NO:24. In some embodiments, the phdC comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:24.

In some embodiments, the yield of benzylamine is greater than 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, 2.0 g/L, 2.1 g/L, 2.2 g/L, 2.3 g/L, 2.4 g/L, 2.5 g/L, 2.6 g/L, 2.7 g/L, 2.8 g/L, 2.9 g/L, 3.0 g/L, 3.1 g/L, 3.2 g/L, or greater than 3.3 g/L.

In some embodiments, the yield of benzylamine is greater than 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, or greater than 31 mM.

In some embodiments, the percentage yield of benzylamine from benzoic acid is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the percentage yield of benzylamine from benzaldehyde is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the percentage conversion of benzylamine from benzoic acid is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the percentage conversion of benzylamine from benzaldehyde is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the method further comprises providing a phosphopantetheinyl transferase (PPTase) and/or an alanine dehydrogenase (AlaDH) activity.

In some embodiments, the method further comprises PPTase and/or an alanine dehydrogenase AlaDH activity.

In some embodiments, the microorganism that expresses the CAR and TA also expresses the PPTase. In some embodiments, the microorganism that expresses the CAR and TA also expresses the AlaDH. In some embodiments, the microorganism that expresses the CAR and TA also expresses the PPTase and the AlaDH.

In some embodiments, the PPTase comprises an amino acid sequence with at least 40% sequence identity to SEQ ID NO:12. In some embodiments, the PPTase comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:12.

In some embodiments, the PPTase is a wild type or mutant Sfp derived from Bacillus subtilis.

In some embodiments, the AlaDH comprises an amino acid sequence with at least 40% sequence identity to SEQ ID NO:13. In some embodiments, the AlaDH comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:13.

In some embodiments, the AlaDH is a wild type or mutant AlaDH derived from Bacillus subtilis.

In some embodiments, the microorganism has been transformed or transfected with a plasmid. In some embodiments the microorganism has been transformed or transfected with a plasmid comprising a CAR gene and/or a TA gene. In some embodiments, the microorganism has been transformed or transfected with a plasmid comprising a CAR gene and another plasmid comprising a TA gene. In some embodiments, the microorganism is transformed or transfected with a plasmid described in the examples. In some embodiments, the microorganism has been transformed or transfected with pBbE2c NiCAR_Sfp and/or pBbA1k CvTA_BsAlaDH (P3B-CvTA). In some embodiments, the microorganism has been transformed or transfected with a plasmid comprising a CAR gene and a PPTase gene and/or a plasmid containing a TA gene and an AlaDH gene.

In some embodiments, the expression of enzymes or genes encoding enzymes is inducible. In some embodiments, the enzymes or genes encoding enzymes are constitutively expressed. In some embodiments, the expression of the CAR, the TA, the PPTase and/or the AlaDH is inducible. In some embodiments, the gene encoding the CAR, the gene encoding the TA, the gene encoding the PPTase, and/or the gene encoding the AlaDH is present on an inducible plasmid. In some embodiments, the production of the CAR and/or TA enzymes is induced by a specific inducer. In some embodiments, the level of production of CAR, the TA, the PPTase and/or the AlaDH enzyme is controlled by the concentration of inducer that is provided. In some embodiments, the inducer is IPTG.

In some embodiments, the expression of enzymes or genes encoding enzymes is induced in the conversion stage. In some embodiments, the expression or production of the CAR, the TA, the PPTase and/or the AlaDH is induced in the conversion stage. In some embodiments, the expression or production of the CAR, the TA, the PPTase and/or the AlaDH is not induced in the growth stage.

In some embodiments, the microorganism comprises a plasmid. In some embodiments, the microorganism comprises a plasmid comprising a CAR gene and/or a TA gene. In some embodiments, the microorganism comprises a plasmid comprising a CAR gene and another plasmid comprising a TA gene. In some embodiments, the microorganism comprises a plasmid described in the examples. In some embodiments, the microorganism comprises pBbE2c NiCAR_Sfp and/or pBbA1k CvTA_BsAlaDH (P3B-CvTA). In some embodiments, the microorganism comprises a plasmid containing a CAR gene and a PPTase gene and/or a plasmid containing a TA gene and an AlaDH gene.

In some embodiments, the method does not include the provision of an amine donor. In some embodiments, the method does not include the provision of an exogenous amine donor. Alanine is an exemplary amine donor. In some embodiments, the method does not include the provision of exogenous alanine.

In some embodiments, an amine donor is provided. An amine donor may alternatively be described as an amino donor. In some embodiments, the amine donor is alanine. In some embodiments, an amine donor is provided to facilitate step (b). In some embodiments, an amine donor is provided for the conversion of the aldehyde into an amine. In some embodiments, an amine donor provides an amino group for the conversion of the aldehyde into an amine. In some embodiments, an amine donor provides an amino group for the conversion of the aldehyde into an amine catalysed by a transaminase (TA).

In some embodiments, exogenous amine is provided at a concentration lower than 250 mM. In some embodiments, exogenous amine is provided a concentration lower than 200 mM, lower than 150 mM, lower than 100 mM, lower than 50 mM, lower than 40 mM, lower than 30 mM, lower than 20 mM, lower than 10 mM, lower than 5 mM, lower than 4 mM, lower than 3 mM, lower than 2 mM, or lower than 1 mM.

In some embodiments, exogenous alanine is provided at a concentration lower than 250 mM. In some embodiments, exogenous alanine is provided at a concentration lower than 200 mM, lower than 150 mM, lower than 100 mM, lower than 50 mM, lower than 40 mM, lower than 30 mM, lower than 20 mM, lower than 10 mM, lower than 5 mM, lower than 4 mM, lower than 3 mM, lower than 2 mM, or lower than 1 mM.

In some embodiments, exogenous glucose is provided. In some embodiments, exogenous glucose is provided in a buffer solution. In some embodiments, exogenous glucose is provided at a concentration lower than 250 mM. In some embodiments, exogenous glucose is provided a concentration lower than 200 mM, lower than 150 mM, lower than 100 mM, lower than 50 mM, lower than 40 mM, lower than 30 mM, lower than 20 mM, lower than 10 mM, lower than 5 mM, lower than 4 mM, lower than 3 mM, lower than 2 mM, or lower than 1 mM.

In some embodiments, exogenous glucose is provided at a concentration greater than 100 mM. In some embodiments, exogenous glucose is provided at a concentration greater than 100 mM, greater than 50 mM, greater than 40 mM, greater than 30 mM, greater than 20 mM, greater than 10 mM, greater than 5 mM, greater than 4 mM, greater than 3 mM, greater than 2 mM, or greater than 1 mM.

In some embodiments, exogenous glucose is provided to facilitate the production of an amine. In some embodiments, exogenous glucose is provided to facilitate the conversion of carboxylic acid to amine. In some embodiments, exogenous glucose is provided to facilitate the conversion of aldehyde to amine.

In some embodiments, exogenous glucose is provided in a resting stage. In some embodiments, exogenous glucose is provided following a growth stage. In some embodiments, exogenous glucose is provided in a resting stage following a growth stage.

In some embodiments, the method comprises biotransformation of carboxylic acid to amine in a buffer with a volume greater than 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 150 ml, 200 ml, 250 ml, 300 ml, 350 ml, 400 ml, 450 ml, 500 ml, 600 ml, 700 ml, 800 ml, 900 ml, 1000 ml, 2000 ml, 3000 ml, 4000 ml, 5000 ml, 6000 ml, 7000 ml, 8000 ml, 9000 ml, or greater than 10000 ml.

In some embodiments, the method comprises biotransformation of benzoic acid to benzylamine in a buffer with a volume greater than 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 150 ml, 200 ml, 250 ml, 300 ml, 350 ml, 400 ml, 450 ml, 500 ml, 600 ml, 700 ml, 800 ml, 900 ml, 1000 ml, 2000 ml, 3000 ml, 4000 ml, 5000 ml, 6000 ml, 7000 ml, 8000 ml, 9000 ml, or greater than 10000 ml.

In some embodiments, the method comprises biotransformation of carboxylic acid to amine in a buffer with a volume less than 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 150 ml, 200 ml, 250 ml, 300 ml, 350 ml, 400 ml, 450 ml, 500 ml, 600 ml, 700 ml, 800 ml, 900 ml, 1000 ml, 2000 ml, 3000 ml, 4000 ml, 5000 ml, 6000 ml, 7000 ml, 8000 ml, 9000 ml, or less than 10000 ml.

In some embodiments, the method comprises biotransformation of benzoic acid to benzylamine in a buffer with a volume less than 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 150 ml, 200 ml, 250 ml, 300 ml, 350 ml, 400 ml, 450 ml, 500 ml, 600 ml, 700 ml, 800 ml, 900 ml, 1000 ml, 2000 ml, 3000 ml, 4000 ml, 5000 ml, 6000 ml, 7000 ml, 8000 ml, 9000 ml, or less than 10000 ml.

In some embodiments, the method comprises biotransformation of carboxylic acid to amine in a buffer with a volume between 1 ml and 2 ml, 1 ml and 3 ml, 1 ml and 4 ml, 1 ml and 5 ml, 1 ml and 6 ml, 1 ml and 7 ml, 1 ml and 8 ml, 1 ml and 9 ml, 1 ml and 10 ml, 1 ml and 20 ml, 1 ml and 30 ml, 1 ml and 40 ml, 1 ml and 50 ml, 1 ml and 60 ml, 1 ml and 70 ml, 1 ml and 80 ml, 1 ml and 90 ml, 1 ml and 100 ml, 1 ml and 150 ml, 1 ml and 200 ml, 1 ml and 250 ml, 1 ml and 300 ml, 1 ml and 350 ml, 1 ml and 400 ml, 1 ml and 450 ml, 1 ml and 500 ml, 1 ml and 600 ml, 1 ml and 700 ml, 1 ml and 800 ml, 1 ml and 900 ml, 1 ml and 1000 ml, 1 ml and 2000 ml, 1 ml and 3000 ml, 1 ml and 4000 ml, 1 ml and 5000 ml, 1 ml and 6000 ml, 1 ml and 7000 ml, 1 ml and 8000 ml, 1 ml and 9000 ml, or between 1 ml and 10000 ml.

In some embodiments, the method comprises biotransformation of carboxylic acid to amine in a buffer with a volume between 5 ml and 6 ml, 5 ml and 7 ml, 5 ml and 8 ml, 5 ml and 9 ml, 5 ml and 10 ml, 5 ml and 20 ml, 5 ml and 30 ml, 5 ml and 40 ml, 5 ml and 50 ml, 5 ml and 60 ml, 5 ml and 70 ml, 5 ml and 80 ml, 5 ml and 90 ml, 5 ml and 100 ml, 5 ml and 150 ml, 5 ml and 200 ml, 5 ml and 250 ml, 5 ml and 300 ml, 5 ml and 350 ml, 5 ml and 400 ml, 5 ml and 450 ml, 5 ml and 500 ml, 5 ml and 600 ml, 5 ml and 700 ml, 5 ml and 800 ml, 5 ml and 900 ml, 5 ml and 1000 ml, 5 ml and 2000 ml, 5 ml and 3000 ml, 5 ml and 4000 ml, 5 ml and 5000 ml, 5 ml and 6000 ml, 5 ml and 7000 ml, 5 ml and 8000 ml, 5 ml and 9000 ml, or between 5 ml and 10000 ml.

In some embodiments, the method comprises biotransformation of carboxylic acid to amine in a buffer with a volume between 500 ml and 600 ml, 500 ml and 700 ml, 500 ml and 800 ml, 500 ml and 900 ml, 500 ml and 1000 ml, 500 ml and 2000 ml, 500 ml and 3000 ml, 500 ml and 4000 ml, 500 ml and 5000 ml, 500 ml and 6000 ml, 500 ml and 7000 ml, 500 ml and 8000 ml, 500 ml and 9000 ml, or between 500 ml and 10000 ml.

In some embodiments, the method comprises biotransformation of carboxylic acid to amine in a buffer with a volume between 1000 ml and 2000 ml, 1000 ml and 3000 ml, 1000 ml and 4000 ml, 1000 ml and 5000 ml, 1000 ml and 6000 ml, 1000 ml and 7000 ml, 1000 ml and 8000 ml, 1000 ml and 9000 ml, or between 1000 ml and 10000 ml.

In a further aspect, a method of producing a compound is provided, wherein the method comprises: (a) the method of producing an amine according to any previous claim; and (b) the production of a compound utilising the amine produced in step (a), preferably wherein the amine produced in step (a) is benzylamine and the compound produced in step (b) is hexanitrohexaazaisowurtzitane (CL-20).

In a further aspect, a method of producing a propellant is provided. In some embodiments, the method comprises a method of producing an amine described in this disclosure, and further comprises the conversion of the amine to a propellant.

In some embodiments, the propellant is hexanitrohexaazaisowurtzitane (CL-20).

In a further aspect, an organism for the production of amine is provided, wherein the microorganism expresses a heterologous TA and a heterologous CAR.

In some embodiments, the organism is a microorganism. In some embodiments, the organism is a bacterium.

The TA is functional. The TA comprises transaminase activity.

In some embodiments, the TA is an omega-TA (ω-TA).

In some embodiments, the ω-TA comprises an amino acid sequence with at least 40% sequence identity to SEQ ID NO: 7, 8, 9, 10, 11 or 25. In some embodiments, the ω-TA comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NOs: 7, 8, 9, 10, 11 or 25. In some embodiments, the ω-TA is a wild type or mutant ω-TA derived from Chromobacterium violaceum or from Vibrio fluvialis.

The CAR is functional. The CAR comprises carboxylic acid reductase activity.

In some embodiments, the CAR comprises an amino acid sequence with at least 40% sequence identity to SEQ ID NO: 1, 2, 3, 4, 5, or 6. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NOs: 1, 2, 3, 4, 5, or 6.

In some embodiments, the CAR is a wild type or mutant CAR derived from Nocardia iowensis.

In some embodiments, the organism has been modified to inhibit aldehyde reduction. In some embodiments, the organism has been modified to reduce or eliminate the activity or expression of one or more aldo-keto reductases (AKRs) and/or alcohol dehydrogenases (ADHs). In some embodiments, the activity or expression of an AKR or ADH enzyme which comprises an amino acid sequence with at least 40% sequence identity to SEQ ID NO:14, 15, 16, 17, 18, or 19 is reduced or eliminated. In some embodiments, the AKR or ADH comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NOs: 14, 15, 16, 17, 18, or 19.

In some embodiments, the organism further comprises phosphopantetheinyl transferase (PPTase) and/or an alanine dehydrogenase (AlaDH).

In some embodiments, the organism that expresses the CAR and TA also expresses the PPTase. In some embodiments, the organism that expresses the CAR and TA also expresses the AlaDH. In some embodiments, the organism that expresses the CAR and TA also expresses the PPTase and the AlaDH.

In some embodiments, the PPTase comprises an amino acid sequence with at least 40% sequence identity to SEQ ID NO: 12. In some embodiments, the PPTase comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:12.

In some embodiments, the PPTase is a wild type or mutant PPTase derived from Bacillus subtilis.

In some embodiments, the AlaDH comprises an amino acid sequence with at least 40% sequence identity to SEQ ID NO:13. In some embodiments, the AlaDH comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NOs: 13.

In some embodiments, the AlaDH is a wild type or mutant AlaDH derived from Bacillus subtilis.

In one aspect, use of an organism as described in this disclosure is envisaged. In some embodiments, use of an organism expressing a heterologous TA and a heterologous CAR in the production of an amine is envisaged. In some embodiments, the use comprises contacting a carboxylic acid or conjugate base with the CAR, and further comprises contacting the aldehyde with the TA. In some embodiments, carboxylic acid is converted to an aldehyde through CAR activity, and the aldehyde is converted to an amine through TA activity.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1: Schematic overview of possible enzymatic cascades for the synthesis of benzoate and benzylamine.

FIG. 2: Diagrammatic overview of possible benzylamine synthesis processes via biotransformation of (A) petrochemical-derived benzoate and (B) biologically-derived benzoate. Diagrams made using BioRender.com.

FIG. 3: Comparison of benzylamine synthesis between E. coli MG1655 and E. coli MG1655 RARE strains each transformed with pBbE2c NiCAR_Sfp and pBbA1k CvTA following the addition of 50 mM benzoic acid to induced biomass.

FIG. 4: Input variables and measured outputs from the Design of Experiments (DoE) guided optimisation of benzoic acid biotransformation.

FIG. 5: Benzylamine titres (A) and yield (B) obtained during the DoE guided biotransformation of benzoic acid.

FIG. 6: JMP Pro 16 DoE outputs for the bioconversion of benzoic acid to benzylamine (A) effect summary for terms included in the Generalised Linear Model (GLM), (B) prediction profiler plot for optimal input variable configuration and (C) interaction profiler plot under the optimal parameter configuration.

FIG. 7: Benzylamine titres from E. coli MG1655 RARE P3B-CvTA grown in baffled shake flasks at 10 mL, 100 mL and 1 L reaction volumes.

FIG. 8: Benzylamine titres from E. coli MG1655 RARE P3B-CvTA and VfTA at varying initial biomass loadings (OD₆₀₀ₙₘ 25, 50 and 100) in the presence and absence of 100 mM alanine.

FIG. 9: Benzylamine titres obtained using freeze-dried E. coli MG1655 RARE P3B-CvTA and VfTA biomass.

FIG. 10: Benzylamine titres for fed-batch fermentative conversion of benzoic acid using a BioLector microbioreactor system. Benzoic acid was added once cultures reached an OD₆₀₀ₙₘ of 18. For wells with multiple doses, addition of benzoic acid was performed at 4 h intervals.

FIG. 11: BioLector outputs for fed-batch fermentative conversion of benzoic acid to benzylamine using E. coli MG1655 RARE. • indicates induction and * indicates benzoic acid feeding. (A) OD₆₀₀ₙₘ for un-induced control, induced control and experimental culture fed 100 mM benzoic acid (B) dissolved oxygen (DO) for experimental culture fed 100 mM benzoic acid and (C) pH for experimental culture fed 100 mM benzoic acid.

FIG. 12: Plasmid map of pBbA1-P3B-CvTA_BsAla_DH_NiCAR_Sfp.

FIG. 13: (A) CAR-TA cascade from benzoic acid to benzylamine, (B) effect of cell line and plasmid content on benzylamine titres from benzoic acid, (C) effect of cell line and plasmid content on benzylamine titres from benzaldehyde, (D) effect of cell line and plasmid content on the side reaction of benzaldehyde to benzoic acid in the absence of exogenous alanine.

FIG. 14: (A) Reaction of AlaDH of oxidative deamination of L-alanine in forward reaction and reductive amination of pyruvate in backward reaction, (B) effect of exogenously added alanine and glucose on benzylamine titre in 5 mL reactions, (C) effect of exogenously added alanine and glucose on benzylamine titre in 10 mL reactions, (D) effect of exogenously added alanine and glucose on benzylamine titre in 50 mL reactions.

FIG. 15: (A) Benzylamine cascade reaction (5 mL) over 24 h with three E. coli strains as whole cell biocatalysts, (B) benzylamine cascade reaction (10 mL) over 24 h with three E. coli strains as whole cell biocatalysts.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present invention is based on the construction of a novel biosynthetic pathway for the production of amines, and novel methods of utilising this novel pathway.

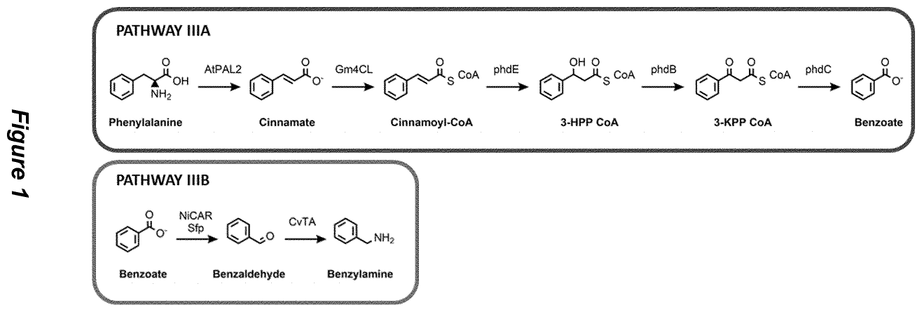

The present inventors have identified a novel biosynthetic route to amines (e.g. benzylamine) from carboxylic acids (e.g. benzoic acid) utilising carboxylic acid reductase (CAR) and w-transaminase (ω-TA) enzymes (FIG. 1) which has produced a benzylamine titre of 3.4 g/L.

Additionally, while biosynthetic routes to amines are known, the present inventors also found that splitting the process into more than one stage (or module), rather than following one continuous pathway, can improve benzylamine yield and improve conversion.

Splitting the method into stages allows the carboxylic acid substrate to be produced at high levels in one stage, biomass to be grown in another stage, and the addition of carboxylic acid to the biomass in a further stage. Producing carboxylic acid separately allows the provision of carboxylic acid at a higher concentration than would otherwise accumulate in cells. The method may comprise a carboxylic acid production stage, a growth stage and/or a conversion stage.

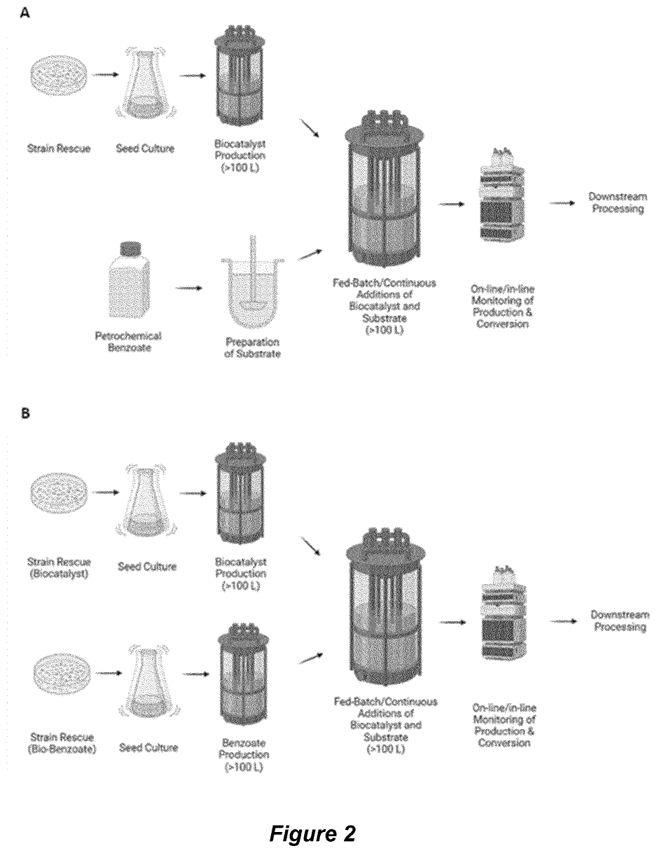

FIG. 2 provides an overview of possible benzylamine synthesis processes utilising chemically-derived substrate with two separate bioreactors (FIG. 2A) and utilising biologically-derived substrate with three separate bioreactors (FIG. 2B). An additional option is to supplement chemically-derived substrate directly into the bioreactor in which the biocatalyst is produced therefore requiring only one bioreactor.

### Amines

Amines are compounds that contain a basic nitrogen atom with a lone pair. Amines are formally derivatives of ammonia (NH₃), wherein one or more hydrogen atoms have been replaced by a substituent such as an alkyl or aryl group.

Amines can be classified according to the nature and number of substituents on nitrogen. Aliphatic amines contain only H and alkyl substituents.

Aromatic amines have the nitrogen atom connected to an aromatic ring. It is a broad class of compounds that encompasses anilines, but also many more complex aromatic rings and many amine substituents beyond NH₂. Such compounds occur widely.

In some embodiments, the amine is a primary amine. Primary amines arise when one of three hydrogen atoms in ammonia is replaced by an alkyl or aromatic group. Important primary alkyl amines include, methylamine, most amino acids, and the buffering agent TRIS, while primary aromatic amines include aniline and benzylamine.

In some embodiments, the amine is an aromatic amine. Aromatic amines are widely used as precursor to pesticides, pharmaceuticals, and dyes. Benzylamine, aniline, toluidines, and phenylenediamine are aromatic amines. In some embodiments, the aromatic amine is benzylamine, aniline, phenylenediamine, and/or toluidine. In some embodiments, the aromatic amine is benzylamine, aniline, and/or toluidine.

Amines may comprise substitutions (e.g., one of the CH₂ groups may be substituted with a different functional group). Substitutions may be performed through downstream processing after an amine is produced. In some embodiments, one of the CH₂ groups of an amine is substituted with a different functional group. In some embodiments, one of the CH₂ groups of an amine is substituted with an alkyl, alkenyl, alkynyl, phenyl, amino, hydroxy, alkoxy, thiol, or amide.

In some embodiments, the amine is an aromatic amine comprising a substitution on the aromatic ring. In some embodiments, the substitution comprises the addition of an alkyl, alkenyl, alkynyl, phenyl, amino, hydroxy, alkoxy, thiol, or amide to the aromatic ring. In some embodiments, the amine is an aromatic amine comprising an alkyl substitution on the aromatic ring. In some embodiments, the amine is an aromatic amine comprising a methyl substitution on the aromatic ring. In some embodiments, the amine is an aromatic amine comprising a hydroxy group on the aromatic ring.

In some embodiments, the amine is an aromatic amine comprising an alkyl group on the aromatic ring. In some embodiments, the amine is an aromatic amine comprising an methyl group on the aromatic ring. In some embodiments, the amine is an aromatic amine comprising a hydroxy group on the aromatic ring.

In some embodiments, the amine is a primary aromatic amine. In some embodiments, the primary aromatic amine is benzylamine, aniline, phenylenediamine, and/or toluidine. In some embodiments, the primary aromatic amine is benzylamine, aniline, and/or toluidine.

In some embodiments, the amine is benzylamine.

Benzylamine has the molecular formula C₇H₉N or C₆H₅CH₂NH₂, and is also known as phenylmethanamine, benzenemethanamine, and monobenzylamine. Benzylamine is a primary amine compound having benzyl as the N-substituent. It is a colourless to light yellow liquid with a strong odour of ammonia, and it floats and mixes with water. Benzylamine is a precursor to the synthesis of many commercially-important compounds including motion-sickness treatments, anticonvulsants and the high-energy propellant hexanitrohexaazaisowurtzitane (CL-20).

In some embodiments, the amine is benzylamine or a substituted benzylamine. In some embodiments, the amine is a substituted benzylamine. In some embodiments, benzylamine is produced through a method herein and subsequently modified to produce a substituted benzylamine. Methods of modifying benzylamine to produce substituted benzylamine, and the use of such substituted benzylamines, are known in the art (e.g., Kramer et al., Org. Lett. 2019, 21, 1, 65-69, and WO2004007457A2), which are hereby incorporated by reference in their entirety). In some embodiments, the substituted benzylamine comprises a an alkyl, alkenyl, alkynyl, phenyl, amino, hydroxy, alkoxy, thiol, or amide substitution.

In some embodiments, the amine is a salt of benzylamine. In some embodiments, the substituted benzylamine is a salt of benzylamine (i.e., a benzylamine salt). Benzylamine salts are substances which are particularly useful as synthetic intermediates for dyestuffs, pesticides, medicines or the like, as well as other various industrial chemicals. Benzylamine salts may be produced from benzylamine through any method known in the art. Crystal structures of seven molecular salts derived from benzylamine and organic acidic components are described by Wen et al. (2017. Journal of Molecular Structure, Volume 1139, p. 87-103), which is hereby incorporated by reference in its entirety. In some embodiments, the benzylamine salt is a hydrochloride salt of benzylamine. The hydrochloride salt of benzylamine, or benzylamine hydrochloride (C₇H₁₀ClN, C₆H₅CH₂NH₃Cl or C₆H₅CH₂NH. HCl), is prepared by reacting benzylamine with hydrochloric acid.

In some embodiments, the amine comprises a single amino group. In some embodiments, the amine is not a diamine, triamine, or tetraamine.

### Carboxylic Acid Substrates

A carboxylic acid substrate is a carboxylic acid, a conjugate base of a carboxylic acid (also known as a carboxylate), a salt of a carboxylic acid (also known as a carboxylate salt) or an ester of a carboxylic acid (also known as a carboxylate ester).

A carboxylic acid is an organic compound containing a carboxyl functional group. They occur widely in nature and can be synthetically manufactured. Upon deprotonation, carboxylic acids yield a carboxylate anion with the general formula R—COO—, which can form a variety of useful salts such as soaps.

In some embodiments, the carboxylic acid is an aromatic carboxylic acid, or an aromatic acid. Aromatic acids include compounds that contain a COOH group bonded to an aromatic ring. Benzoic acid, salicylic acid, gallic acid, toluic acid, phthalic acid, isophthalic acid, and terephthalic acid are aromatic carboxylic acids. The simplest aromatic acid is benzoic acid.

In some embodiments, the carboxylic acid is benzoic acid.

Benzoic acid, C₆H₅COOH, is a colourless crystalline solid and the simplest aromatic carboxylic acid. Benzoic acid occurs naturally free and bound as benzoic acid esters in many plant and animal species. Benzoate is the conjugate base of benzoic acid, comprising a benzoic acid core with a proton missing to give a negative charge (⁻¹).

In some embodiments, the conjugate base of a carboxylic acid is benzoate.

### Aldehydes

Aldehydes, like ketones, contain a carbonyl functional group, C═O. Specifically, aldehydes comprise a carbon connected by a double bond to oxygen (carbonyl), a single bond to hydrogen, and single bond to a third substituent.

In some embodiments, the aldehyde is an aromatic aldehyde. Aromatic aldehydes include compounds that comprise a carbon connected by a double bond to oxygen (carbonyl), a single bond to hydrogen, and single bond to an aromatic ring. Benzaldehyde and phenylethanone (acetophenone) are examples of aromatic amines.

In some embodiments, the aldehyde is benzaldehyde.

Benzaldehyde, C₇H₆O or C₆H₅CHO, is an arenecarbaldehyde that consists of benzene bearing a single formyl substituent; the simplest aromatic aldehyde and parent of the class of benzaldehydes.

### Carboxylic Acid Reductase (CAR) Enzymes

CAR enzymes comprise enzymatic activity which facilitates the conversion of carboxylic acids (e.g. benzoic acid) to aldehydes (e.g. benzaldehydes). Individual CAR enzymes, e.g. the Nocardia iowensis CAR, are capable of catalysing the reduction of a wide range of carboxylic acids.

Two enzyme classes are able to reduce carboxylic acids to aldehydes. The aldehyde oxidoreductases (AORs) oxidize organic aldehydes reversibly to their respective acids. The oxidized product is more thermodynamically favorable, and so the equilibrium tends towards this product. Therefore, AORs are more useful for syntheses that require the oxidation of aldehydes. In contrast, CARs catalyze the reduction of a carboxylic acid to an aldehyde at the expense of adenosine triphosphate (ATP) and NADPH to produce adenosine monophosphate (AMP), pyrophosphate (PPi), and NADP+ as byproducts (Finnegan et al., 2017).

CARs are relatively large, multidomain enzymes of around 130 kDa. They feature an N-terminal adenylation domain, a C-terminal thioester reductase domain that likely adopts a Rossmann fold, and a central phosphopantetheine binding domain (Marchler-Bauer et al., 2015. Nucleic Acids Res, 43, D222-D2226).

The CAR comprises carboxylic acid reductase activity. This activity can be assayed through any method known in the art, for example those described in Finnegan et al., 2017, Khusnutdinova et al., Biotechnol J. 2017 November; 12(11): 10.1002/biot.201600751, and/or Marchler-Bauer et al., 2015. Nucleic Acids Res, 43, D222-D2226. To summarise, carboxylic acid reductase activity of purified CARs against different carboxylic acids can be determined spectrophotometrically using an NADPH oxidation-based assay by following the decrease in absorbance at 340 nm. In one example of this assay, a reaction mixture (0.2 ml) containing HEPES-K (100 mM, pH 7.5), 1 mM NADPH, 2.5 mM ATP, 10 mM MgCl₂, 10 mM substrate (e.g. benzoic acid or decanoic acid), and 2.5-5.0 μg of purified CAR (10 min incubation at 30° C.) could be employed to assay CAR activity.

The methods of the present disclosure employ microorganisms which express a CAR. In some embodiments, the CAR is a wild type or mutant CAR derived from a species of the following group of genera: Nocardia, Mycobacterium, Streptomyces, Segniliparus, or Tsukamurella.

In some embodiments, the CAR is a wild type or mutant CAR derived from Nocardia iowensis, Mycobacterium phlei, Mycobacterium smegmatis, Mycobacterium marinum, Nocardia otitidiscaviarum, or Tsukamurella paurometabola.

In some embodiments, the CAR is a wild type or mutant CAR derived from Nocardia iowensis, Mycobacterium phlei, Mycobacterium smegmatis, Nocardia otitidiscaviarum, or Tsukamurella paurometabola.

In some embodiments, the CAR is a wild type or mutant CAR derived from Nocardia iowensis or Mycobacterium marinum. In some embodiments, the CAR is a wild type or mutant CAR derived from Nocardia iowensis. In some embodiments, the CAR is a wild type or mutant CAR derived from Mycobacterium marinum. In some embodiments, the CAR is a Nocardia iowensis CAR. In some embodiments, the CAR is a Mycobacterium marinum CAR.

In some embodiments, the CAR comprises an amino acid sequence with at least 40% sequence identity to the amino acid sequence of the Nocardia iowensis CAR (UniProt Q6RKB1). In some embodiments, the CAR comprises an amino acid sequence with at least 40% sequence identity to SEQ ID NO:1. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:1.

The Nocardia iowensis CAR catalyzes the reduction of a very wide range of aliphatic carboxylic acids as well as many aryl carboxylic acids. Aryl carboxylic acid substrates include substituted benzoic acids, phenyl-substituted aliphatic acids, heterocyclic carboxylic acids, and polyaromatic ring carboxylic acids.

In some embodiments, the CAR comprises an amino acid sequence with at least 40% sequence identity to the amino acid sequence of the Mycobacterium phlei CAR (UniProt: A0A5N5VEC4). In some embodiments, the CAR comprises an amino acid sequence with at least 40% sequence identity to SEQ ID NO: 2. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:2.

In some embodiments, the CAR comprises an amino acid sequence with at least 40% sequence identity to the amino acid sequence of the Mycobacterium smegmatis CAR1 (UniProt: A0A653FAQ0). In some embodiments, the CAR comprises an amino acid sequence with at least 40% sequence identity to SEQ ID NO: 3. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:3.

In some embodiments, the CAR comprises an amino acid sequence with at least 40% sequence identity to the amino acid sequence of the Mycobacterium smegmatis CAR2 (UniProt: A0A653FCI4). In some embodiments, the CAR comprises an amino acid sequence with at least 40% sequence identity to SEQ ID NO: 4. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:4.

In some embodiments, the CAR comprises an amino acid sequence with at least 40% sequence identity to the amino acid sequence of the Nocardia otitidiscaviarum CAR (UniProt: A0A516NPD0). In some embodiments, the CAR comprises an amino acid sequence with at least 40% sequence identity to SEQ ID NO: 5. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:5.

In some embodiments, the CAR comprises an amino acid sequence with at least 40% sequence identity to the amino acid sequence of the Tsukamurella paurometabola CAR (UniProt: D5UWX9). In some embodiments, the CAR comprises an amino acid sequence with at least 40% sequence identity to SEQ ID NO: 6. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:6.

In some embodiments, the CAR comprises an amino acid sequence with at least 40% sequence identity to the amino acid sequence of the Mycobacterium marinum CAR (UniProt: B2HN69). In some embodiments, the CAR comprises an amino acid sequence with at least 40% sequence identity to SEQ ID NO: 28. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:28.

### Transaminase (TA) Enzymes

TAs, also known as aminotransferases, are a group of enzymes that mediate the transfer of an amine group between an amino acid and a keto acid. TA enzymes are capable of the enzymatic conversion of an aldehyde (e.g. benzaldehyde) to an amine (e.g. benzylamine). It has been shown that ω-transaminases (ω-TAs) can be efficient enzymes for the conversion of aldehyde to amine (Fuchs et al., 2012).

The methods of the present disclosure employ microorganisms which express a TA. In some embodiments, the TA is an ω-TA. In some embodiments, the TA is a wild type or mutant TA derived from a species of the following group of genera: Chromobacterium, Vibrio, Alcaligenes, Pseudomonas, or Paracoccus. In some embodiments, the TA is a wild type or mutant TA derived from Chromobacterium violaceum, Vibrio fluvialis, Alcaligenes denitrificans (also known as Achromobacter denitrificans), Pseudomonas aeruginosa, or Paracoccus denitrificans. In some embodiments, the TA is a wild type or mutant TA derived from Chromobacterium violaceum or Vibrio fluvialis.

The TA comprises transaminase activity. This activity can be assayed through any transaminase activity assay that is known in the art. For example, the method of Smith and Taylor (J. clin. Path., 1973, 26, 42-47), or Fuchs et al. (2012) may be employed. Alternatively, commercially available enzyme assay kits can be employed, such as those made available by Abcam (Alanine Transaminase Activity Assay Kit (ab105134/K752-100)) or ScienCell (Alanine Transaminase Assay, Catalog #8478, ScienCell Research Laboratories). In one example, an assay is based on the oxidization of NADH to NAD in the presence of pyruvate and lactate dehydrogenase. The transaminase activity is determined by assaying the rate of NADH oxidation, which is proportional to the reduction in absorbance at 340 nm over time (ΔOD₃₄₀ nm/min).

In some embodiments, the TA comprises an amino acid sequence with at least 40% sequence identity to the amino acid sequence of the Chromobacterium violaceum TA (CvTA; UNIPROT:Q7NWG4). In some embodiments, the TA comprises an amino acid sequence with at least 40% sequence identity to SEQ ID NO: 7. In some embodiments, the TA comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:7.

In some embodiments, the TA comprises an amino acid sequence with at least 40% sequence identity to the amino acid sequence of the Vibrio fluvialis TA (VfTA; UNIPROT:F2XBU9). In some embodiments, the TA comprises an amino acid sequence with at least 40% sequence identity to SEQ ID NO:8. In some embodiments, the TA comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:8.

In some embodiments, the TA comprises an amino acid sequence with at least 40% sequence identity to the amino acid sequence of the Alcaligenes denitrificans TA (AdTA; UNIPROT:Q7WWK8). In some embodiments, the TA comprises an amino acid sequence with at least 40% sequence identity to SEQ ID NO: 9. In some embodiments, the TA comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:9.

In some embodiments, the TA comprises an amino acid sequence with at least 40% sequence identity to the amino acid sequence of the Pseudomonas putida TA (PpTA; UNIPROT:Q88KV9). In some embodiments, the TA comprises an amino acid sequence with at least 40% sequence identity to SEQ ID NO: 10. In some embodiments, the TA comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:10.

In some embodiments, the TA comprises an amino acid sequence with at least 40% sequence identity to the amino acid sequence of the Paracoccus denitrificans TA (PdTA; UNIPROT:A1B956). In some embodiments, the TA comprises an amino acid sequence with at least 40% sequence identity to SEQ ID NO: 11. In some embodiments, the TA comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:11.

In some embodiments, the TA comprises an amino acid sequence with at least 40% sequence identity to the amino acid sequence of the Pseudomonas aeruginosa TA (PaTA; UNIPROT:Q9I6J). In some embodiments, the TA comprises an amino acid sequence with at least 40% sequence identity to SEQ ID NO: 25. In some embodiments, the TA comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:25.

### Phosphopantetheinyl Transferase (PPTase) Enzymes

Phosphopantetheinyl transferases (PPTases) are essential for cell viability across all three domains of life: bacteria, archaea and eukaryota. These enzymes are known to the skilled person, and reviewed, for example, in Beld et al. Nat Prod Rep. 2014 January; 31 (1): 61-108.

PPTases are utilised in this disclosure to enhance the loading of a phosphopantetheine group onto CAR enzymes. Any PPTase may be suitable for this function.

In some embodiments, the method comprises the provision of an PPTase. In some embodiments, the method comprises PPTase activity.

In some embodiments, the PPTase is expressed by a microorganism, comprised within a cell lysate, or is provided exogenously, e.g. in a purified form.

In some embodiments, the PPTase is a microbial PPTase. In some embodiments, the PPTase is a bacterial PPTase, or mutant PPTase derived from a bacterium.

In some embodiments, the PPTase is a wild type or mutant surfactin phosphopantetheinyl transferase (Sfp) derived from Bacillus subtilis.

The PPTase comprises phosphopantetheinyl transferase activity. This activity can be assayed through any method that is known in the art. For example, phosphopantetheinyl transferase activity can be assayed through the methods highlighted in Owen et al. (Biochem J. 2011 Jun. 15; 436(3):709-17. doi: 10.1042/BJ20110321). In one embodiment, an indigoidine-synthesizing non-ribosomal peptide synthetase BpsA is utilised as a reporter for PPTase activity.

In some embodiments, the PPTase comprises an amino acid sequence with at least 40% sequence identity to the amino acid sequence of the Bacillus subtilis Sfp (Uniprot: P39135). In some embodiments, the PPTase comprises an amino acid sequence with at least 40% sequence identity to SEQ ID NO: 12. In some embodiments, the PPTase comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:12.

### Alanine Dehydrogenase (AlaDH) Enzymes

AlaDH (E.C.1.4.1.1) is an enzyme that catalyzes a reversible conversion of L-alanine to pyruvate. Inter-conversion of alanine and pyruvate by AlaDH is central to metabolism in microorganisms. These enzymes are known to the skilled person, and reviewed, for example, by Dave and Kadeppagari, (Crit Rev Biotechnol. 2019 August; 39(5):648-664. doi: 10.1080/07388551.2019.1594153).

The use of AlaDH is the present disclosure is one way in which amine groups can be provided for the conversion of aldehydes to amines. Specifically, alanine can be recycled from pyruvate via (formal) reductive amination using AlaDH enzymes. The use of AlaDH, or similar enzymes, can therefore prevent or reduce the need to provide exogenous amine, such as exogenous alanine.

In some embodiments, the method comprises the provision of an enzyme with AlaDH activity. In some embodiments, the method comprises the provision of an AlaDH. In some embodiments, the method comprises AlaDH activity.

As highlighted above, AlaDH activity is the catalysation of a reversible conversion of L-alanine to pyruvate. In some embodiments, AlaDH activity increases amine yield. In some embodiments, AlaDH activity increases the amine yield of a method of producing an amine, compared to a method of producing an amine which does not comprise AlaDH activity.

In some embodiments, the use of an AlaDH enzyme increases amine yield. In some embodiments, the use of an AlaDH enzyme increases the amine yield of a method of producing an amine, compared to a method of producing an amine which does not comprise the use of an AlaDH enzyme.

In some embodiments, the AlaDH is expressed by a microorganism, comprised within a cell lysate, or is provided exogenously, e.g. in a purified form.

In some embodiments, the AlaDH is a microbial AlaDH. In some embodiments, the AlaDH is a bacterial AlaDH, or mutant AlaDH derived from a bacterium.

In some embodiments, the AlaDH is a wild type or mutant AlaDH derived from Bacillus subtilis.

The AlaDH comprises alanine dehydrogenase activity. This activity can be assayed through any method known in the art. For example, activity of an AlaDH can be assayed by incubating the AlaDH with pyruvate and monitoring the level of alanine generated. Alternatively, the method described by Bae et al. (2003. J. Microbiol. Biotechnol. 13(4), 628-631) could be used.

In some embodiments, the AlaDH comprises an amino acid sequence with at least 40% sequence identity to the amino acid sequence of the Bacillus subtilis AlaDH (Uniprot: Q08352). In some embodiments, the AlaDH comprises an amino acid sequence with at least 40% sequence identity to SEQ ID NO: 13. In some embodiments, the AlaDH comprises, or consists, of an amino acid sequence having at least 45%, 50%, 55%, 60%, 65%, 70%, 75%, 80%, 85%, 90%, 95%, 99%, or 100% sequence identity to the amino acid sequence of SEQ ID NO:13.

### Methods of Producing Amines

The present disclosure provides methods of producing an amine, comprising the provision of a microorganism and the provision of a carboxylic acid, wherein the microorganism expresses a carboxylic acid reductase (CAR) and a transaminase (TA), and the carboxylic acid is converted to an aldehyde through CAR activity, and the aldehyde is converted to an amine through TA activity.

Specific methods of producing an amine according to the present disclosure are disclosed within the examples herein. Additionally, the chemical and biochemical components of the method have been discussed in previous sections.

The method may comprise more than one stage. For example, the method may comprise one or more of a carboxylic acid production stage, a growth stage, and a conversion stage.

The method may comprise more than one method of growing or utilising a microorganism. The method may comprise more than one substrate conversion process. The method may comprise fermentation, whole cell biocatalysis, biotransformation, and/or product isolation.

In some embodiments, the method of producing an amine comprises whole cell biocatalysis. In some embodiments, the method of producing an amine is a whole cell biocatalysis method of producing an amine.

Whole cell biocatalysis is reviewed in detail in Lin and Tao (Microbial Cell Factories volume 16, Article number: 106 (2017)). Whole-cell catalysis approaches can broadly be classified into biotransformation (biocatalysis) and fermentation bioprocesses. In fermentations, the products are synthesized from growth substrates via the host cells' native metabolism and are accompanied in the fermentation broth by metabolic intermediates that make downstream processing complicated. In biotransformations, cell growth (the enzyme manufacturing phase) and production phase (conversion stage) are separated.

Substrates are converted to the desired products by resting cells. The key advantages of whole-cell biocatalysis are its abilities to use cheap and abundant raw materials and to catalyse multistep reactions.

The method may involve culture or fermentation of a microorganism. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted or expressed peptide or polypeptide. Culture, fermentation and separation techniques are well known to those of skill in the art, and are described, for example, in Green and Sambrook, Molecular Cloning: A Laboratory Manual (4th Edition; incorporated by reference herein above).

In some examples, a fed batch method is utilised. In some examples, a fed batch fermentation is utilised. In some embodiments, a dose of carboxylic acid (or more than one dose) is provided during microorganism growth. In some embodiments, a dose of carboxylic acid (or more than one dose) is provided during exponential microorganism growth. In some embodiments, a dose of carboxylic acid (or more than one dose) is provided during mid-late exponential microorganism growth.

Cultures of a microorganism, or microbial cultures, or cultures or microbiological cultures, generally comprise a culture vessel, a growth medium, and at least one microorganism cell. In some embodiments, the culture is a lab-scale culture. In some embodiments, the culture is an industrial-scale culture. In some embodiments of the present disclosure, the microorganism cell is a bacterial cell.

A microbial medium, microbial growth medium or microbial culture medium is a liquid, semi-solid or solid designed to support the growth and proliferation of microbial cells. Microbial culture media are well known by scientists in the area of cell culture. Microbial cell culture media types and methods are comprehensively reviewed in A Rouf, Varsha Kanojia, H R Naik, Bazilla Naseer and Tahiya Qadri (2017) An overview of microbial cell culture, Journal of Pharmacognosy and Phytochemistry, Vol. 6, Issue 6 p 1923-1928, the contents of which are incorporated by reference.

Microbial culture media may comprise an appropriate source of energy and compounds which regulate the cell cycle. In addition to nutrients, the medium also helps maintain pH and osmolality. However, in some embodiments, there is no glucose and/or no carbon source present in the media.

Microbial culture media may be liquid (i.e. aqueous), semi-solid (i.e. gelatinous), or completely solid. Semi-solid and solid media may contain agar, silicagel, acrylamide, gellan gum, or other solidification agents. Liquid media generally do not contain solidification agents.

In some embodiments, the culture media is Luria broth (LB), high salt Luria broth (LB60), minimal media, high salt minimal media, or another suitable media known by the skilled person.

LB Miller contains 5 g/L yeast extract, 10 g/L tryptone and 10 g/L NaCl pH 7.0, and is widely used and well known to the skilled person. High salt Luria broth (LB60) is identical to LB, but has a higher salt content (60 g/L NaCl). Minimal medium is culture medium for microorganisms that contains the minimal necessities for growth-containing only inorganic salts, water, and optionally a carbon source. High salinity minimal medium contains higher salt content than typical minimal medium. An exemplary high salinity minimal medium is filter sterilised sea water with or without a supplemental carbon source.

In some embodiments, the microbial culture media is taken directly from the environment, for example from sea water, brackish water, river water, lake water, pond water. In some embodiments the cells are grown directly in the water taken from the environment, in some embodiments the environmental water is processed (e.g. filtered and/or autoclaved) before use as culture media, in some embodiments further additives (e.g. carbon, mineral, and/or amino acids) are added to the water to support growth of the cells. In some embodiments, the water is polluted with excess carbon and/or excess sulfur containing compounds. The sulfur containing compound may be a sulfate, a thiosulfate, and/or a sulfite.

In some embodiments, an amine donor is provided. An amine donor may alternatively be described as an amino donor. In some embodiments, the amine donor is alanine. In some embodiments, an amine donor is provided for the conversion of the aldehyde into an amine. In some embodiments, an amine donor provides an amino group for the conversion of the aldehyde into an amine. In some embodiments, an amine donor provides an amino group for the conversion of the aldehyde into an amine catalysed by a transaminase (TA).

In some embodiments, the method does not include the provision of an amine donor. In some embodiments, the method does not include the provision of an exogenous amine donor.

In some embodiments, the amine donor is alanine. In some embodiments, the method does not comprise the provision of exogenous alanine.

In some embodiments, an exogenous amine donor is provided at a concentration lower than 250 mM. In some embodiments, an exogenous amine donor is provided at a concentration lower than 200 mM, lower than 150 mM, lower than 100 mM, lower than 50 mM, lower than 40 mM, lower than 30 mM, lower than 20 mM, lower than 10 mM, lower than 5 mM, lower than 4 mM, lower than 3 mM, lower than 2 mM, or lower than 1 mM.

In some embodiments, exogenous alanine is provided at a concentration lower than 250 mM. In some embodiments, exogenous alanine is provided at a concentration lower than 200 mM, lower than 150 mM, lower than 100 mM, lower than 50 mM, lower than 40 mM, lower than 30 mM, lower than 20 mM, lower than 10 mM, lower than 5 mM, lower than 4 mM, lower than 3 mM, lower than 2 mM, or lower than 1 mM.

In some embodiments, exogenous glucose is provided. In some embodiments, exogenous glucose is provided at a concentration lower than 250 mM. In some embodiments, exogenous glucose is provided at a concentration lower than 200 mM, lower than 150 mM, lower than 100 mM, lower than 50 mM, lower than 40 mM, lower than 30 mM, lower than 20 mM, lower than 10 mM, lower than 5 mM, lower than 4 mM, lower than 3 mM, lower than 2 mM, or lower than 1 mM.

In some embodiments, exogenous glucose is provided at a concentration greater than 100 mM, lower than 50 mM, lower than 40 mM, lower than 30 mM, lower than 20 mM, lower than 10 mM, lower than 5 mM, lower than 4 mM, lower than 3 mM, lower than 2 mM, or lower than 1 mM.

In some embodiments, exogenous glucose is provided to facilitate the production of an amine. In some embodiments, exogenous glucose is provided to facilitate the production of benzylamine. In some embodiments, exogenous glucose is provided to facilitate the conversion of carboxylic acid to amine. In some embodiments, exogenous glucose is provided to facilitate the conversion of aldehyde to amine.

In some embodiments, exogenous glucose is provided in a conversion stage. In some embodiments, exogenous glucose is provided following a growth stage. In some embodiments, exogenous glucose is provided in a conversion stage following a growth stage.

### Growth Stage

In some embodiments, the method of producing an amine comprises a growth stage.

In some embodiments, the carboxylic acid substrate is provided exogenously to the microorganism while the microorganism is in a growth stage.

The growth stage, also described as a growth phase or growth process, is the stage of the method of producing an amine in which growth of the microorganism occurs.

In some embodiments, the microorganism is grown in a microbial growth medium.

The growth stage may involve culture or fermentation of cells modified to express the relevant polypeptide(s). The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted or expressed peptide or polypeptide. Culture, fermentation and separation techniques are well known to those of skill in the art, and are described, for example, in Green and Sambrook, Molecular Cloning: A Laboratory Manual (4th Edition; incorporated by reference herein), and Doran, Chapter 1—Bioprocess Development: An Interdisciplinary Challenge, Bioprocess Engineering Principles (Second Edition; incorporated by reference herein) 2013, Pages 3-11.

In some embodiments, the growth stage comprises culture or fermentation of a microorganism. The culture or fermentation may be performed in a bioreactor provided with an appropriate supply of nutrients, air/oxygen and/or growth factors. Culture, fermentation and separation techniques are well known to those of skill in the art, and are described, for example, in Green and Sambrook, Molecular Cloning: A Laboratory Manual (4th Edition; incorporated by reference herein), and Doran, Chapter 1—Bioprocess Development: An Interdisciplinary Challenge, Bioprocess Engineering Principles (Second Edition; incorporated by reference herein) 2013, Pages 3-11. Any culture vessel or media, for example those described in this disclosure, can be used in the growth stage.

In some embodiments, the growth stage occurs until the culture reaches a certain density, or the culture comprises a specific amount of biomass.

The biomass of a culture can be accurately estimated using optical density (OD) assays.

In some embodiments, microorganisms are moved from the growth stage to the conversion stage when OD at a wavelength of 600 nm (OD₆₀₀ₙₘ) is above a pre-specified threshold. In some embodiments, microorganisms are grown until the culture reaches an OD₆₀₀ₙₘ of at least 0.1. In some embodiments, microorganisms are grown until the culture reaches an OD₆₀₀ₙₘ of at least 0.2, 0.3, 0.4, 0.5, 0.6, 0.7, 0.8, 0.9, or at least 1.0.

### Conversion Stage

In some embodiments, the carboxylic acid substrate is provided exogenously to the microorganism while the microorganism is in a resting stage following a growth stage.

A resting stage may also be known as a conversion stage.

In some embodiments, microorganisms are moved from a growth stage to a conversion stage. In some embodiments, microorganisms are moved from the culture media to a buffer. In some embodiments, microorganisms are moved from the culture media in the growth stage to a buffer for the conversion stage. In some embodiments, a buffer is a solution containing either a weak acid and its salt or a weak base and its salt, which is resistant to significant changes in pH within a certain range (buffer capacity).

When moving microorganisms from a culture media, the solid cell biomass must be separated from the liquid media. The media is a liquid fraction or a liquid portion which lies above a sediment formed by the solid microorganisms. The microorganism fraction is the solid sediment fraction which forms below the liquid fraction. The microorganism fraction can be separated from the liquid fraction in a number of ways known by the skilled person, including filtration, chromatography, evaporation, sedimentation, and centrifugation.

In many known methods of whole cell biocatalysis, microorganisms are freeze dried (lyophilised) after the growth stage. In some embodiments, microorganisms are lyophilised.

Inventors have found that lyophilisation can have a negative impact on amine yields (shown in Example 6). In some embodiments, microorganisms are not lyophilised. In some embodiments, the microorganism is fresh. In some embodiments, the microorganism is hydrated. In some embodiments, the microorganism is non-lyophilised. In some embodiments, the microorganism is viable. In some embodiments, the microorganism is not lyophilised before the conversion stage. In some embodiments, the microorganism is re-hydrated in an aqueous liquid following lyophilisation.

Following separation from the media (culture media, liquid fraction, liquid portion), the microorganisms are resuspended in a liquid for the conversion stage. In some embodiments, the liquid for the conversion stage is a buffer. In some embodiments, the buffer is phosphate-citrate buffer (see Example 1 for an exemplar recipe). In some embodiments, the microorganisms are washed before resuspension.

Microorganisms can be resuspended at a concentration that is higher than is typically possible through fermentation methods. The effect of biomass loading is considered in Example 5 of this disclosure.

The biomass of a culture can be accurately estimated using optical density (OD) assays, e.g. OD at a wavelength of 600 nm (OD₆₀₀ₙₘ).

In some embodiments, the microorganism is resuspended at a cell density wherein an OD₆₀₀ₙₘ measurement is above or below a pre-specified threshold.

In some embodiments, the microorganism is resuspended at a cell density wherein an OD₆₀₀ₙₘ measurement is above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or above 100.

In some embodiments, the microorganism is resuspended at a cell density wherein an OD₆₀₀ₙₘ measurement is below 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or below 100.

After resuspension, the microorganisms are utilised in a method of biotransformation.

In some embodiments, carboxylic acid is provided in the conversion stage.

The inventors have found that carboxylic acid can be provided in a number of ways. In some embodiments, carboxylic acid is provided directly in the conversion stage. In some embodiments, the carboxylic acid is petrochemical derived carboxylic acid. In some embodiments, the carboxylic acid is carboxylic acid produced by a different organism.

The inventors have also found that providing carboxylic acid in the conversion stage can increase amine yields. Providing carboxylic acid in the conversion stage enables high concentrations of carboxylic acid to be provided, e.g. at levels much higher than the microorganism could produce themselves. This enables a large amount of carboxylic acid to be processed through the microorganism in the conversion stage.

In some embodiments, the carboxylic acid is provided exogenously to the microorganism. In some embodiments, the carboxylic acid is exogenous carboxylic acid. In some embodiments, the benzoic acid is provided exogenously to the microorganism. In some embodiments, the benzoic acid is exogenous benzoic acid.

In some embodiments, the conjugate base is provided exogenously to the microorganism. In some embodiments, the conjugate base is exogenous conjugate base. In some embodiments, the benzoate is provided exogenously to the microorganism. In some embodiments, the benzoate is exogenous benzoate.

In some embodiments, the carboxylic acid (e.g. benzoic acid) is provided in an isolated or purified form. In some embodiments, the carboxylic acid is provided as a mixture. In some embodiments, the carboxylic acid is provided at a purity of at least 50%. In some embodiments, the carboxylic acid is provided at a purity of at least 55%, 60%, 65%, 70%, 75%, 80%, 85%, 90%, 95%, 96%, 97%, 98%, or at least 99%.

In some embodiments, carboxylic acid (e.g benzoic acid) is provided at a concentration of less than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or less than 200 mM.

In some embodiments, carboxylic acid (e.g. benzoic acid) is provided at a concentration of less than 200 mM. In some embodiments, carboxylic acid (e.g. benzoic acid) is provided at a concentration of less than 150 mM. In some embodiments, carboxylic acid (e.g. benzoic acid) is provided at a concentration of less than 100 mM.

In some embodiments, carboxylic acid (e.g. benzoic acid) is provided at a concentration of more than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or more than 200 mM.

In some embodiments, carboxylic acid (e.g. benzoic acid) is provided at a concentration of more than 10 mM. In some embodiments, carboxylic acid (e.g. benzoic acid) is provided at a concentration of more than 20 mM. In some embodiments, carboxylic acid (e.g. benzoic acid) is provided at a concentration of more than 30 mM. In some embodiments, carboxylic acid (e.g. benzoic acid) is provided at a concentration of more than 40 mM. In some embodiments, carboxylic acid (e.g. benzoic acid) is provided at a concentration of more than 50 mM.

In some embodiments, carboxylic acid (e.g. benzoic acid) is provided at a concentration of ≥10 mM, ≥20 mM, ≥30 mM, ≥40 mM, ≥50 mM, ≥60 mM, ≥70 mM, ≥80 mM, ≥90 mM, ≥100 mM, ≥110 mM, ≥120 mM, ≥130 mM, ≥140 mM, ≥150 mM, ≥160 mM, ≥170 mM, ≥180 mM, ≥190 mM, or ≥200 mM. In some embodiments, carboxylic acid (e.g. benzoic acid) is provided at a concentration of ≥50 mM.

In some embodiments, carboxylic acid (e.g. benzoic acid) is provided at a concentration of ≤10 mM, ≤20 mM, ≤30 mM, ≤40 mM, ≤50 mM, ≤60 mM, ≤70 mM, ≤80 mM, ≤90 mM, ≤100 mM, ≤110 mM, ≤120 mM, ≤130 mM, ≤140 mM, ≤150 mM, ≤160 mM, ≤170 mM, ≤180 mM, ≤190 mM, or ≤200 mM. In some embodiments, carboxylic acid (e.g. benzoic acid) is provided at a concentration of ≤50 mM.

In some embodiments, carboxylic acid (e.g. benzoic acid) is provided at a concentration of more than 10 mM and less than 150 mM. In some embodiments, carboxylic acid (e.g. benzoic acid) is provided at a concentration of more than 10 mM and less than 100 mM. In some embodiments, carboxylic acid (e.g benzoic acid) is provided at a concentration of more than 10 mM and less than 90 mM. In some embodiments, carboxylic acid (e.g. benzoic acid) is provided at a concentration of more than 10 mM and less than 80 mM. In some embodiments, carboxylic acid (e.g. benzoic acid) is provided at a concentration of more than 10 mM and less than 70 mM. In some embodiments, carboxylic acid (e.g. benzoic acid) is provided at a concentration of more than 10 mM and less than 60 mM. In some embodiments, carboxylic acid (e.g. benzoic acid) is provided at a concentration of more than 10 mM and less than 50 mM.

In some embodiments, carboxylic acid (e.g. benzoic acid) is provided at a starting concentration of less than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or less than 200 mM. In some embodiments, carboxylic acid (e.g. benzoic acid) is provided at a starting concentration of less than 100 mM.

In some embodiments, carboxylic acid (e.g. benzoic acid) is provided at a starting concentration of more than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or more than 200 mM. In some embodiments, carboxylic acid (e.g. benzoic acid) is provided at a starting concentration of more than 10 mM.

In some embodiments, carboxylic acid (e.g. benzoic acid) is provided at a starting concentration of more than 10 mM and less than 150 mM. In some embodiments, carboxylic acid (e.g. benzoic acid) is provided at a starting concentration of more than 10 mM and less than 100 mM. In some embodiments, carboxylic acid (e.g. benzoic acid) is provided at a starting concentration of more than 10 mM and less than 90 mM. In some embodiments, carboxylic acid (e.g. benzoic acid) is provided at a starting concentration of more than 10 mM and less than 80 mM. In some embodiments, carboxylic acid (e.g. benzoic acid) is provided at a starting concentration of more than 10 mM and less than 70 mM. In some embodiments, carboxylic acid (e.g. benzoic acid) is provided at a starting concentration of more than 10 mM and less than 60 mM. In some embodiments, carboxylic acid (e.g. benzoic acid) is provided at a starting concentration of more than 10 mM and less than 50 mM.

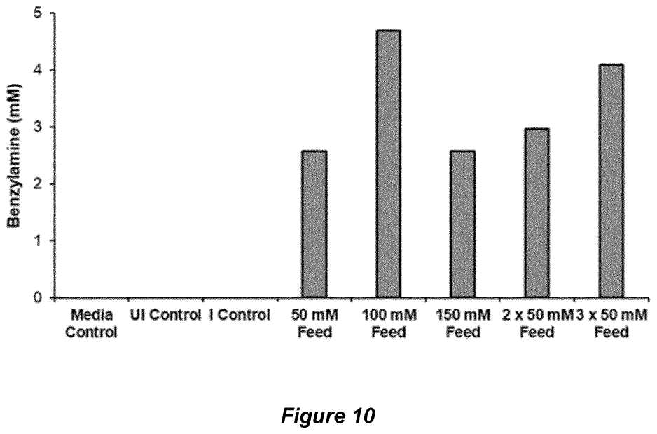

The inventors have investigated the effect of increasing the number of carboxylic acid doses. In some cases, increasing the dose can increase the yield of amine (e.g. benzylamine). For example, it is shown in FIG. 10 that benzylamine yield is considerably higher with three 50 mM doses (feeds) compared to a single 50 mM carboxylic acid dose.

In some embodiments, one dose of carboxylic acid (e.g benzoic acid) is provided. In some embodiments, multiple doses of carboxylic acid (e.g benzoic acid) are provided. In some embodiments, 2, 3, 4, 5, 6, 7, 8, 9, or 10 doses of carboxylic acid (e.g benzoic acid) are provided. In some embodiments, each dose of carboxylic acid (e.g benzoic acid) is provided at a concentration of less than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or less than 200 mM. In some embodiments, each dose of carboxylic (e.g benzoic acid) acid is provided at a concentration of more than 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or more than 200 mM. In some embodiments, each dose of carboxylic acid (e.g benzoic acid) is provided at a concentration of 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or 200 mM.

In some embodiments, amine production occurs within the cell of the microorganism. In some embodiments, amine biosynthesis occurs within the cell of the microorganism. In some embodiments, conversion of carboxylic acid to amine occurs within the cell of the microorganism. In some embodiments, conversion of carboxylic acid to aldehyde occurs within the cell of the microorganism. In some embodiments, conversion of aldehyde to amine occurs within the cell of the microorganism.

In some embodiments, amine production occurs within the buffer or cell lysate. In some embodiments, amine biosynthesis occurs within the buffer or cell lysate. In some embodiments, conversion of carboxylic acid to amine occurs within the buffer or cell lysate. In some embodiments, conversion of carboxylic acid to aldehyde occurs within the buffer or cell lysate. In some embodiments, conversion of aldehyde to amine occurs within the buffer or cell lysate.

In some embodiments, the carboxylic acid or conjugate base is contacted with the CAR within the cell of the microorganism. In some embodiments, the aldehyde is contacted with the TA within the cell of the microorganism.

In some embodiments, the carboxylic acid or conjugate base is contacted with the CAR within the buffer or cell lysate. In some embodiments, the aldehyde is contacted with the TA within the buffer or cell lysate.

Cell lysate comprises a liquid component (e.g. media or a buffer) and the contents of lysed cells (e.g. proteins such as enzymes).

### Carboxylic Acid Substrate Production

There are numerous ways in which carboxylic acid substrates can be produced.

Within this document, methods of carboxylic acid production should be understood to also include methods of producing conjugate bases of carboxylic acids, for example, methods of benzoic acid production include methods of benzoate production.

The present disclosure is compatible with all methods of carboxylic acid production.

This disclosure encompasses methods of producing an amine, comprising the provision of a microorganism and the provision of a carboxylic acid or the conjugate base of a carboxylic acid.

In some embodiments, there is a carboxylic acid production stage. In some embodiments, the carboxylic acid production stage and the growth stage are performed separately. In some embodiments, the carboxylic acid production stage and the growth stage are performed separately and simultaneously. In some embodiments, the carboxylic acid production stage is performed before the growth stage.

The carboxylic acid or the conjugate base of a carboxylic acid can be provided from any source. The carboxylic acid can be purchased from a supplier or can be produced by a person using the method of this disclosure. The carboxylic acid can be produced chemically, industrially, biologically, or through any other method known by the skilled person.

Carboxylic acids can be produced chemically from petrochemical sources, for example by oxidising primary alcohols or aldehydes. Primary alcohols and aldehydes are normally oxidised to carboxylic acids using potassium dichromate(VI) solution in the presence of dilute sulphuric acid. This process is not renewable or energy efficient, but is convenient for end-users.

Carboxylic acids can also be produced biochemically by organisms, such as microorganisms, which comprise suitable enzymatic activities. Biosynthetic routes to benzoic acid have previously been disclosed (Luo & Lee, 2020; Zhou et al., 2020), the contents of these publications are incorporated in their entirety. One example of an enzymatic pathway for the production of a carboxylic acid is shown in Pathway IIIA of FIG. 1.

In some embodiments, the organism is capable of producing benzoic acid. In some embodiments, the organism is capable of producing benzoic acid from phenylalanine. In some embodiments, the organism is capable of producing benzoic acid from glucose.

In some embodiments, the organism capable of producing benzoic acid comprises the enzymes capable of producing benzoic acid from phenylalanine. In some embodiments, the organism capable of producing benzoic acid from phenylalanine comprises a phenylalanine ammonia lyase, a coumarate-CoA-ligase, and phenylpropanoid degradation pathway enzymes. In some embodiments, the organism capable of producing benzoic acid from phenylalanine comprises the enzymes shown in Pathway IIIA of FIG. 1.

In some embodiments, the organism capable of producing benzoic acid comprises an enzyme comprising an amino acid sequence with at least 40% sequence identity to the amino acid sequence of Arabidopsis thaliana phenylalanine ammonia-lyase 2 (PAL2; AtPAL2) (UniProt: P45724). In some embodiments, the enzyme comprises an amino acid sequence with at least 40% sequence identity to SEQ ID NO:20. In some embodiments, the AtPAL2 comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:20.

In some embodiments, the organism capable of producing benzoic acid comprises an enzyme comprising an amino acid sequence with at least 40% sequence identity to the amino acid sequence of Glycine max 4-coumarate: CoA ligase (4CL; Gm4CL) (UniProt: Q8S5C2). In some embodiments, the enzyme comprises an amino acid sequence with at least 40% sequence identity to SEQ ID NO:21. In some embodiments, the Gm4CL comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:21.

In some embodiments, the organism capable of producing benzoic acid comprises one or more Corynebacterium glutamicum phd enzymes, variants of Corynebacterium glutamicum phd enzymes, or enzymes which are homologous to Corynebacterium glutamicum phd enzymes. In some embodiments, the organism capable of producing benzoic acid comprises one or more of Corynebacterium glutamicum phdB (3-hydroxyacyl-CoA dehydrogenase), Corynebacterium glutamicum phdC (3-oxoacyl-CoA ketohydrolase) (acetyl-CoA forming), or Corynebacterium glutamicum phdE (enoyl-CoA hydratase).

In some embodiments, the organism capable of producing benzoic acid comprises an enzyme capable of converting cinnamoyl-CoA to 3-HPP CoA. In some embodiments, the organism capable of producing benzoic acid comprises an enzyme comprising an amino acid sequence with at least 40% sequence identity to the amino acid sequence of phdE from Corynebacterium glutamicum. In some embodiments, the organism capable of producing benzoic acid comprises an enzyme comprising an amino acid sequence with at least 40% sequence identity to the amino acid sequence of phdE from Corynebacterium glutamicum (GENBANK ID: AGN20978.1). In some embodiments, the enzyme comprises an amino acid sequence with at least 40% sequence identity to SEQ ID NO:22. In some embodiments, the phdE comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:22.

In some embodiments, the organism capable of producing benzoic acid comprises an enzyme capable of converting 3-HPP CoA to 3-KPP CoA. In some embodiments, the organism capable of producing benzoic acid comprises an enzyme comprising an amino acid sequence with at least 40% sequence identity to the amino acid sequence of phdB from Corynebacterium glutamicum. In some embodiments, the organism capable of producing benzoic acid comprises an enzyme comprising an amino acid sequence with at least 40% sequence identity to the amino acid sequence of phdB from Corynebacterium glutamicum (GENBANK ID: AGN20975). In some embodiments, the enzyme comprises an amino acid sequence with at least 40% sequence identity to SEQ ID NO:23. In some embodiments, the phdB comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO: 23.

In some embodiments, the organism capable of producing benzoic acid from phenylalanine comprises an enzyme capable of converting 3-KPP CoA to benzoic acid. In some embodiments, the organism capable of producing benzoic acid from phenylalanine comprises an enzyme which comprises an amino acid sequence with at least 40% sequence identity to the amino acid sequence of phdC from Corynebacterium glutamicum. In some embodiments, the organism capable of producing benzoic acid from phenylalanine comprises an enzyme which comprises an amino acid sequence with at least 40% sequence identity to the amino acid sequence of phdC from Corynebacterium glutamicum (GENBANK ID: AGN20976.1). In some embodiments, the enzyme comprises an amino acid sequence with at least 40% sequence identity to SEQ ID NO:24. In some embodiments, the phdC comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:24.

In some embodiments, the organism capable of producing a carboxylic acid is a microorganism. In some embodiments, the organism capable of producing benzoic acid from phenylalanine is a microorganism. In some embodiments, the organism is a bacterium, fungus, a cyanobacterium, or an alga. In some embodiments, the microorganism is a gram-positive bacterium. In some embodiments, the microorganism is a gram-negative bacterium.

In some embodiments, the bacterium is selected from the genus Escherichia, Listeria, Clostridium, Staphylococcus, Streptococcus, Pseudomonas, Helicobacter, Neisseria, Legionella, Halomonas, Klebsiella or Yersinia. In some embodiments, the bacterium is Escherichia coli.

### Microorganisms

Aspects of the present invention relate to methods of producing amines, wherein microorganisms are utilised in said methods.

Microorganisms may be provided in isolated form and/or in culture. Microorganisms may be provided in vitro.

A microorganism comprising an enzyme according to the present invention may do so through expression from a nucleic acid/expression vector according to the present invention that has been introduced into the microorganism.

Microorganisms contemplated for use with the present invention include prokaryotic and eukaryotic cells. For example, the prokaryotic cell may be a bacteria or archaea, and the eukaryotic microorganism may be a fungi, protist, or microscopic animal or microscopic plant organism.

In preferred aspects, the microorganism is a bacterium. Any bacterium may be used, such as laboratory strains (such as E. coli or Bacillus subtilis), or field strains. Preferred bacteria will be those that are organotrophic, e.g. chemoheterotrophic bacteria, capable of using biomass or compounds derived therefrom as an energy source.

In particular embodiments, Escherichia bacteria such as E. coli, Saccharomyces yeast such as S. cerevisiae and cyanobacteria are contemplated for use in the present invention.

Preferred bacteria are robust bacteria, such as soil bacteria and/or extremophilic bacteria. Especially preferred are halophilic bacteria. These are capable of growing in open non-sterile conditions. As these strains are salt tolerant, they will not be outcompeted by contaminating bacteria or other microorganisms so long as there is a high enough salt content. Furthermore, the addition of a high salt buffer (e.g. at least a 3% salt solution) can be used to control competing bacteria. Halophilic bacteria include those of the genus Halomonas.

The microorganism may be modified to inhibit aldehyde reduction. The microorganism may be modified to inhibit/reduce/prevent the conversion of aldehydes to alcohols. For example, the microorganism may be modified to inhibit/reduce/prevent the conversion of aldehydes to alcohols by endogenous enzyme activity.

The microorganism may be modified to suppress, inhibit, knock-down, knock-out, and/or mutate endogenous enzymes or the genes encoding enzymes with activity which converts aldehydes to alcohols.

The suppression or inhibition may be partial. Preferred degrees of suppression/inhibition are at least 50%, more preferably one of at least 60, 70, 80, 85 or 90%. A level of suppression between 90% and 100% is considered a ‘silencing’ of expression.

The microorganism may be modified through any known gene silencing methodology. For example, the microorganism may be modified through methodologies involving, siRNA, miRNA, shRNA, TALEN, CRISPR and/or RNAi.

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNAs are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present invention provides the use of these sequences for down-regulating the expression of genes encoding enzymes with activity which converts aldehydes to alcohols.

Methods of RNAi are known in the art, and are described in: Hannon, G. J. 2002. RNA interference. Nature. 418:244-51.

CRISPR (clustered regularly interspaced short palindromic repeats)-Cas (CRISPR associated) systems are prokaryotic adaptive immune system that bind and cleave foreign nucleic acids. The most frequently used type II CRISPR system is composed of two components: Cas9 nuclease and an artificial single guide RNA (sgRNA), a fusion of a CRISPR RNA (crRNA) and a trans-activating crRNA (tracrRNA). When the SpCas9-sgRNA complex recognizes an NGG (N=A, T, C, or G) protospacer-adjacent motif (PAM) sequence, the spacer of the sgRNA pairs with the target DNA strand to form an “R-loop” structure. Subsequently, the Cas9 nuclease cleaves the DNA strands and produces a blunt-end DSB 3 bp upstream of the PAM into the protospacer. CRISPR-Cas gene editing tools are flexible, highly efficient, and inexpensive and have been widely applied. Recently, various Cas orthologs and variants with useful additional properties have been identified and harnessed for use in gene editing. Moreover, novel tools for precise gene modification, such as base editors (BEs) and prime editors (PEs), have greatly expanded the applications of gene editing and have been leveraged for use in a variety of fields of research. Methods of CRISPR-Cas gene editing are reviewed in Liu et al. Mol Cell. 2022 Jan. 20; 82(2):333-347. doi: 10.1016/j.molcel.2021.12.002, and Kantor et al. Int J Mol Sci. 2020 Aug. 28; 21(17):6240. doi: 10.3390/ijms21176240.

In some embodiments, the microorganism has been modified to suppress, inhibit, reduce, or eliminate the activity or expression of one or more aldehyde reductases, aldo-keto reductases (AKRs) and/or alcohol dehydrogenases (ADHs). In some embodiments, the microorganism has been modified to suppress, inhibit, reduce, or eliminate the activity or expression of one or more transcriptional regulators.

In some embodiments, the microorganism has been modified to suppress, inhibit, reduce, or eliminate the activity or expression of one or more of the following enzymes: dkgA, dkgB, yeaE, yahK, yqhD and yjgB, or enzymes with homology to dkgA, dkgB, yeaE, yahK, yqhD and yjgB.

In some embodiments, the microorganism has been modified to suppress, inhibit, reduce, or eliminate the activity or expression of one or more of the following enzymes: yqhC, yqhD, yahK, yeaE, dkgA and yjgB or enzymes with homology to yqhC, yqhD, yahK, yeaE, dkgA and yjgB.

In some embodiments, the microorganism is an E. coli bacterium which has been modified to suppress, inhibit, reduce, or eliminate the activity or expression of one or more aldehyde reductases, aldo-keto reductases (AKRs) and/or alcohol dehydrogenases (ADHs). In some embodiments, the E. coli bacterium has been modified to suppress, inhibit, reduce, or eliminate the activity or expression of one or more transcriptional regulators.

In some embodiments, the transcriptional regulator is yqhC. YqhC regulates transcription of the adjacent E. coli genes yqhD and dkgA.

In some embodiments, the microorganism is an E. coli bacterium which has been modified to suppress, inhibit, reduce, or eliminate the activity or expression of one or more transcriptional regulators, and has been modified to express a heterologous gene described herein.

In some embodiments, the microorganism is an Escherichia coli MG1655 Reduced Aromatic Aldehyde Reduction (RARE) bacterium. The Escherichia coli MG1655 RARE strain is described and utilised in the Examples herein, and is further described in Kunjapur et al. (J Am Chem Soc. 2014 Aug. 20; 136(33):11644-54), which is hereby incorporated by reference in its entirety.

In some embodiments, the microorganism is an Escherichia coli MG1655 Reduced Aromatic Aldehyde Reduction (RARE) strain which has been modified to express a heterologous gene described herein. In some embodiments, the microorganism is an Escherichia coli MG1655 Reduced Aromatic Aldehyde Reduction (RARE) strain which has been modified to express a heterologous CAR and a heterologous TA. In some embodiments, the microorganism is an Escherichia coli MG1655 Reduced Aromatic Aldehyde Reduction (RARE) strain which has been modified to express a heterologous CAR, a heterologous TA, and a heterologous PPTase. In some embodiments, the microorganism is an Escherichia coli MG1655 Reduced Aromatic Aldehyde Reduction (RARE) strain which has been modified to express a heterologous CAR, a heterologous TA, and a heterologous AlaDH. In some embodiments, the microorganism is an Escherichia coli MG1655 Reduced Aromatic Aldehyde Reduction (RARE) strain which has been modified to express a heterologous CAR, a heterologous TA, a heterologous AlaDH, and a heterologous PPTase.

In some embodiments, the microorganism is an E. coli BL21(DE3) BZE bacterium. The E. coli BL21 (DE3) BZE strain is described and utilised in the Examples herein, and is further described in Zhao et al. (ACS Sustainable Chem. Eng. 2021, 9, 18, 6400-6409), which is hereby incorporated by reference in its entirety.

In some embodiments, the microorganism is an E. coli BL21(DE3) BZE strain which has been modified to express a heterologous gene described herein. In some embodiments, the microorganism is an E. coli BL21(DE3) BZE strain which has been modified to express a heterologous CAR and a heterologous TA. In some embodiments, the microorganism is an E. coli BL21(DE3) BZE strain which has been modified to express a heterologous CAR, a heterologous TA, and a heterologous PPTase. In some embodiments, the microorganism is an E. coli BL21(DE3) BZE which has been modified to express a heterologous CAR, a heterologous TA, and a heterologous AlaDH. In some embodiments, the microorganism is an E. coli BL21(DE3) BZE strain which has been modified to express a heterologous CAR, a heterologous TA, a heterologous AlaDH, and a heterologous PPTase.

In some embodiments, the microorganism has been modified to suppress, inhibit, reduce, or eliminate the activity or expression of an enzyme with at least 40% sequence identity to the amino acid sequence of E. coli 2,5-diketo-D-gluconic acid reductase A (DKGA, dgkA) (UniProt: Q46857). In some embodiments, the DGTA comprises an amino acid sequence with at least 40% sequence identity to SEQ ID NO:14. In some embodiments, the DGTA comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO: 14.

In some embodiments, the microorganism has been modified to suppress, inhibit, reduce, or eliminate the activity or expression of an enzyme with at least 40% sequence identity to the amino acid sequence of E. coli 2,5-diketo-D-gluconic acid reductase B (DKGB, dgkB) (Uniprot: P30863). In some embodiments, the DKGB comprises an amino acid sequence with at least 40% sequence identity to SEQ ID NO:15. In some embodiments, the DKGB comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:15.

In some embodiments, the microorganism has been modified to suppress, inhibit, reduce, or eliminate the activity or expression of an enzyme with at least 40% sequence identity to the amino acid sequence of E. coli yeaE (UniProt: A0A0Q2YWH0). In some embodiments, the yeaE comprises an amino acid sequence with at least 40% sequence identity to SEQ ID NO:16. In some embodiments, the yeaE comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO: 16.

In some embodiments, the microorganism has been modified to suppress, inhibit, reduce, or eliminate the activity or expression of an enzyme with at least 40% sequence identity to the amino acid sequence of E. coli aldehyde reductase (YAHK, YahK) (UniProt: P75691). In some embodiments, the YAHK comprises an amino acid sequence with at least 40% sequence identity to SEQ ID NO:17. In some embodiments, the YAHK comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:17.

In some embodiments, the microorganism has been modified to suppress, inhibit, reduce, or eliminate the activity or expression of an enzyme with at least 40% sequence identity to the amino acid sequence of E. coli yghD (UniProt: Q46856). In some embodiments, the yghD comprises an amino acid sequence with at least 40% sequence identity to SEQ ID NO:18. In some embodiments, the yghD comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:18.

In some embodiments, the microorganism has been modified to suppress, inhibit, reduce, or eliminate the activity or expression of an enzyme with at least 40% sequence identity to the amino acid sequence of E. coli aldehyde reductase (AHR, Ahr, YJGB, yjgB) (UniProt: P27250). In some embodiments, the yjgB comprises an amino acid sequence with at least 40% sequence identity to SEQ ID NO: 19. In some embodiments, the yjgB comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:19.

In some embodiments, the microorganism has been modified to suppress, inhibit, reduce, or eliminate the activity or expression of an enzyme with at least 40% sequence identity to the amino acid sequence of the E. coli transcriptional regulator yqhD (Uniprot: A0A0H2Z2M4). In some embodiments, the yqhD comprises an amino acid sequence with at least 40% sequence identity to SEQ ID NO:27. In some embodiments, the yqhD comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:27.

In some embodiments, the microorganism is E. coli, and the E. coli has been modified to reduce or eliminate the activity or expression of one or more of the following enzymes: dkgA, dkgB, yeaE, yahK, yqhD and yjgB. In some embodiments, the microorganism is the E. coli MG1655 RARE strain (Kunjapur et al., dx.doi.org/10.1021/ja506664a|J. Am. Chem. Soc. 2014, 136, 11644-11654).

In some embodiments, the microorganism is E. coli, and the E. coli has been modified to reduce or eliminate the activity or expression of one or more of the following enzymes: yqhC, yqhD, yahK, yeaE, dkgA and yjgB. In some embodiments, the microorganism is the E. coli BL21(DE3) BZE strain (Zhao et al. ACS Sustainable Chem. Eng. 2021, 9, 18, 6400-6409).

### Isolating Amines

In some embodiments, amines are present in the media and are isolated from the liquid fraction (e.g. media or buffer). In other embodiments, amines are isolated from a microorganism fraction. In some embodiments, amines are isolated from the liquid fraction and/or from microorganisms.

The first step for isolation methods is usually to separate the microorganism fraction from the liquid fraction. The liquid fraction is the liquid portion (e.g. culture media or buffer) which lies above a sediment formed by the solid microorganisms. The microorganism fraction is the solid sediment fraction which forms below the liquid fraction.

The microorganism fraction can be separated from the liquid fraction in a number of ways, including filtration, chromatography, evaporation, sedimentation, and centrifugation. Following this crude separation, the compounds of interest can be separated from their fraction.

Compounds that are secreted into the media from microorganisms and present in the liquid fraction can be separated in a number of ways, including physical separation, distillation and pervaporation. For example, the separation of amines from the other components of the media liquid fraction is essentially the separation of ethanol from water with the addition of impurities.

Some amines (e.g. benzylamine) are not soluble in water and therefore naturally separate from water and are easily removed from liquid fractions. In some embodiments, amines are physically separated from other components of the liquid fraction. In some embodiments, the amine is substantially pure after separation from other components of the liquid fraction.

Distillation is the process of separating the components or substances from a liquid mixture by using selective boiling and condensation. The process takes advantage of the fact that different compounds have different boiling points. There are a number of different types of distillation that could be used to separate carbon compounds from media liquid fraction: simple distillation, fractional distillation, vacuum distillation, and azeotropic distillation, to name a few.

Compounds of interest (e.g. amines) which are not secreted, and need to be isolated from the microorganism fraction, require an additional processing step to isolate the compound. To make the compound available for separation, the cells may need to be lysed. Cells can be lysed through mechanical homogenization, ultrasonic homogenisation, pressure homogenisation, freeze-thaw treatment, heat treatment, osmotic lysis and chemical lysis. Following this, the compounds can be isolated through methods known in the art.

Remaining cell mass from the microorganism fraction can be harvested and used as either fertiliser or in animal feeds, or as feedstock for other biotechnological processes. These products are often high in nutrients, minerals, protein, oil, and/or carbohydrates, and have value as fertiliser or in animal feeds. The remaining cell mass can be either whole cells or the solid fraction of lysed cells.

### Yields and Percentage Conversion

The yield, (also referred to as titre, or reaction yield) is the amount of product obtained in a reaction or enzymatic process. The absolute yield can be given as the weight in grams or in moles (molar yield). The fractional yield or relative yield, which serve to measure the effectiveness of a synthetic procedure, is calculated by dividing the amount of the obtained product in moles by the theoretical yield in moles. To obtain a percentage yield, the fractional yield is simply multiplied by 100.

Zhou et al. (2018) discloses biosynthetic routes to benzylamine under biotransformation conditions from L-phenylalanine and under fed-batch fermentation conditions from glucose (obtaining a titre of 0.46 g/L from glucose). Pandey et al. (2021) discloses a shorter biosynthetic route to benzylamine from phenylpyruvate but this did not improve titres (0.024 g/L).

The present method has been shown to produce an amine yield of over 3.3 g/L (over 31 mM).

The present method has been shown to produce benzylamine yield of over 3.3 g/L. This is over 7.3 times higher than the yield demonstrated by Zhou et al. (2018) and over 141.6 times higher than the yield of Pandey et al. (2021).

In some embodiments, the yield of benzylamine is greater than 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, 2.0 g/L, 2.1 g/L, 2.2 g/L, 2.3 g/L, 2.4 g/L, 2.5 g/L, 2.6 g/L, 2.7 g/L, 2.8 g/L, 2.9 g/L, 3.0 g/L, 3.1 g/L, 3.2 g/L, or greater than 3.3 g/L.

In some embodiments, the yield of benzylamine is greater than 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, or greater than 31 mM.

In some embodiments, the percentage yield of benzylamine from benzoic acid is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the percentage yield of benzylamine from benzaldehyde is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In chemistry and biochemistry, and within this document, conversion refers to the conversion of molecule A to molecule B, or molecule A to molecule C (via molecule B). This can be calculated in a similar way to percentage yield.

In the present disclosure, the conversion of aldehyde to amine, and the conversion of carboxylic acid (or conjugate base) to benzylamine (via benzaldehyde), are of interest.

In some embodiments, the conversion of benzaldehyde to benzylamine, and the conversion of benzoic acid to benzylamine (via benzaldehyde), are of interest.

In some embodiments, the percentage conversion of aldehyde to amine is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the percentage conversion of carboxylic acid to amine is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the percentage conversion of benzaldehyde to benzylamine is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

In some embodiments, the percentage conversion of benzoic acid to benzylamine is greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99%.

### Methods of Producing Compositions and Compounds

Amines, such as benzylamine, are commodity chemicals used in the production of numerous products.

In one aspect, the present invention provides a method of producing a composition, wherein the method comprises: (a) any method of producing an amine disclosed herein; and (b) the production of a composition comprising the amine produced in step (a).

In some embodiments, the amine produced in step (a) and comprised within the composition produced in step (b) is benzylamine.

In one aspect, the present invention provides a method of producing a compound, wherein the method comprises: (a) any method of producing an amine disclosed herein; and (b) the production of a compound utilising the amine produced in step (a).

The amine produced in step (a) can be utilised in step (b) as a pre-cursor and/or as an intermediate.

In some embodiments, the amine produced in step (a) is benzylamine.

In some embodiments, step (b) may comprise one or more steps to produce the compound. When step (b) comprises more than one step to produce the compound, either one step, a sub-set of the steps or all of the steps of step (b) may directly utilise the amine.

In some embodiments, the compound produced in step (b) may be one or more of: any compound other than the amine produced in step (a), any compound that can be produced from an amine, any compound that can be produced from the amine produced in step (a), a fuel, an explosive, a propellant and/or CL-20.

In embodiments wherein the amine produced in step (a) is benzylamine, the compound produced in step (b) may be one or more of: any compound other than benzylamine, any compound that can be produced from benzylamine, a fuel, an explosive, a propellant and/or CL-20.

In embodiments wherein the compound produced in step (b) is CL-20, step (b) may comprise any method known in the art of producing CL-20 utilising an amine or utilising benzylamine. In some embodiments, step (b) may comprise any method of producing CL-20 disclosed in Shang et al. (ACS Omega 2022, 7, 25, 21912-21924) or in Nair et al. (Combustion, Explosion and Shock Waves volume 41, pages 121-132 (2005)).

Hexanitrohexaazaisowurtzitane is also known as 2,4,6,8,10,12-hexanitro-2,4,6,8,10,12-hexaazaisowurtzitane, HNW or CL-20. CL-20 is a powerful high energy material and has potential use as an explosive or propellant Nair et al. (Combustion, Explosion and Shock Waves volume 41, pages 121-132 (2005)). CL-20 is a polycyclic nitroamine explosive with the formula C6H6N12O12. In the 1980s, CL-20 was developed by the China Lake facility, primarily to be used in propellants. It has a better oxidizer-to-fuel ratio than conventional HMX or RDX. It releases 20% more energy than traditional HMX-based propellants, and is widely superior to conventional high-energy propellants and explosives.

CL-20 can only be synthesized using an amine with a benzyl group. Shang et al. (ACS Omega 2022, 7, 25, 21912-21924) report that the benzyl group can reduce the energy of the intermediate and the energy barrier of the rate-determining step due to the π-π stack interaction between two benzene rings of the benzyl group.

In one aspect, the present invention provides a method of producing a composition, wherein the method comprises: (a) any method of producing an amine disclosed herein; (b) the production of a compound utilising the amine produced in step (a); and (c) the production of a composition comprising the compound produced in step (b).

The amine produced in step (a), the method of step (b) and the compound produced by step (b) may be as disclosed above.

In some embodiments, the composition produced in step (c) is a fuel composition, an explosive composition and/or a propellant composition. In some embodiments, the composition comprises CL-20.

### Products

In some aspects, the present invention provides: an amine produced according to any method disclosed herein, benzylamine produced according to any method disclosed herein, a compound produced by any method utilising an amine disclosed herein, a composition comprising an amine produced according to any method disclosed herein, a composition comprising a compound produced by any method utilising an amine disclosed herein or CL-20 produced according to any method disclosed herein.

### Nucleic Acids, Expression Vectors, Cells and Compositions

The present invention also provides a nucleic acid, or a plurality of nucleic acids, encoding an enzyme such as a CAR, TA, PPTase, and/or AlaDH.

In some embodiments the nucleic acid is DNA. In some embodiments the nucleic acid is RNA. The nucleic acid may be single-stranded or double-stranded. The nucleic acid may be provided in isolated/purified form, or within a host cell.

In general, short polynucleotides can be produced by synthetic means, involving a stepwise manufacture of the desired nucleic acid sequence one nucleotide at a time. Techniques for accomplishing this using automated techniques are readily available in the art. Longer polynucleotides will generally be produced using recombinant means, for example using PCR (polymerase chain reaction) cloning techniques. In some embodiments this will involve making a pair of primers (e.g. of about 15-30 nucleotides) to a region of the gene which it is desired to clone, bringing the primers into contact with DNA, performing a polymerase chain reaction under conditions which bring about amplification of the desired region, isolating the amplified fragment (e.g. by separating the reaction mixture on an agarose gel) and recovering and purifying the amplified DNA.

The primers may be designed to contain suitable restriction enzyme recognition sites so that the amplified DNA can be cloned into a suitable cloning vector. Although in general the techniques mentioned herein are well known in the art, reference may be made in particular to Sambrook et al., 2001, Molecular Cloning: a laboratory manual, 3rd edition, Cold Harbour Laboratory Press. Alternatively, InFusion cloning (described e.g. in Throop and LaBaer, Curr Protoc Mol Biol. (2015) 110: 3.20.1-3.20.23) or other cloning techniques may be used, such as Gibson Assembly (Gibson et al., Nat. Methods 2009; 6, 343-345), CRISPR/Cas9-based methods (Wang et al., (2015) BioTechniques 58:161-170), Sequence and Ligation Independent Cloning (SLIC; Nucleic Acids Res. 2012, 40: e55) and Modular Overlap-Directed Assembly with Linkers (MODAL; Nucleic Acids Res. (2014) 42.1: e7-e7).

The present invention further provides a vector, particularly an expression vector, comprising a nucleic acid or plurality of nucleic acids according to the present invention. The vector may be used to replicate the nucleic acid in a compatible host cell. Therefore, nucleic acids according to the present invention can be produced by introducing a polynucleotide into a replicable vector, introducing the vector into a compatible host cell and growing the host cell under conditions that bring about replication of the vector.

A “vector” as used herein is an oligonucleotide molecule (DNA or RNA) used as a vehicle to transfer foreign genetic material into a cell. The vector may be an expression vector for expression of the foreign genetic material in the cell. Such vectors may include a promoter and/or a ribosome binding site (RBS) sequence operably linked to the nucleotide sequence encoding the sequence to be expressed. A vector may also include a termination codon and expression enhancers. Such expression vectors are routinely constructed in the art of molecular biology and may for example involve the use of plasmid DNA and appropriate initiators, promoters, RBS, enhancers and other elements, such as for example polyadenylation signals, which may be necessary and which are positioned in the correct orientation in order to allow for protein expression.

Any suitable vectors, promoters, enhancers and termination codons known in the art may be used to express a polypeptide from a vector according to the invention. In some embodiments, the vector may be a plasmid, phage, MAC, virus, etc.

In some embodiments the vector may be a prokaryotic expression vector, e.g. a bacterial expression vector. In some embodiments the vector is a pBbE2c vector or a pBbA1k vector.

In some embodiments, the vector may be a eukaryotic expression vector. In some embodiments, the vector may be a eukaryotic expression vector, e.g. a vector comprising the elements necessary for expression of protein from the vector in a eukaryotic cell. In some embodiments, the vector may be a mammalian expression vector, e.g. comprising a cytomegalovirus (CMV) or SV40 promoter to drive protein expression.

Other suitable vectors would be apparent to persons skilled in the art. By way of further example in this regard we refer to Sambrook et al., 2001, Molecular Cloning: a laboratory manual, 3rd edition, Cold Harbour Laboratory Press.

The term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. The resulting transcript may then be translated into a desired peptide or polypeptide. The promoter may be a T7 promoter.

In some embodiments, the vector may comprise an element for facilitating translation of encoded protein from mRNA transcribed from the construct. For example, the construct may comprise a ribosomal binding site (RBS) such as a Shine-Dalgarno (SD) sequence upstream of the start codon.

In some embodiments, the vector may encode one or more response elements for modulating expression of the encoded protein(s). In some embodiments, the response element is an element that causes upregulation of gene or protein expression in response to treatment with a particular agent. For example, the agent may induce transcription of DNA encoding the protein(s) from a vector including a response element for the agent. In some embodiments the agent may be isopropyl β-D-1-thiogalactopyranoside (IPTG), and the vector may comprise a lac operator. Other induction agent/response element combinations are known in the art.

In some embodiments, the vector may encode one or more response elements for constitutive expression of the encoded protein(s), such that no induction is necessary.

In some embodiments the vector may comprise a transcription terminator sequence downstream of the sequences encoding to the protein or proteins of interest. In some embodiments the terminator may be a T7 terminator sequence. In some embodiments the vector may comprise a sequence encoding a detectable marker in-frame with the sequence encoding the protein of interest to facilitate detection of expression of the protein, and/or purification or isolation of the protein (e.g. a His, (e.g. 6×His), Myc, GST, MBP, FLAG, HA, E, or Biotin tag, optionally at the N- or C-terminus).

In some embodiments, nucleic acids (e.g., genes) disclosed herein are provided within a plasmid for the production of enzymes described herein (e.g., a CAR, TA, PPTase, and/or AlaDH) in a host cell. In some embodiments, the plasmid is a plasmid described herein. In some embodiments, the plasmid is a plasmid described in the Examples herein.

In some embodiments, the plasmid encodes a CAR and/or a TA. In some embodiments, the plasmid encodes a CAR. In some embodiments, the plasmid encodes a TA.

In some embodiments, the plasmid encodes a CAR, TA, PPTase, and/or AlaDH. In some embodiments, the plasmid comprises nucleic acids encoding a CAR and a PPTase. In some embodiments, the plasmid comprises nucleic acids encoding a TA and an AlaDH. In some embodiments, the plasmid encodes a CAR, TA, PPTase, and AlaDH.

In some embodiments, genes are provided in a two-plasmid system. A two-plasmid system is defined herein as two plasmids which together comprise the genes necessary for the expression of an enzymatic cascade. For example, a two-plasmid system for the production of an amine may comprise the genes necessary for the expression of (i) a CAR and a TA, (ii) a CAR, a TA, and a PPTase, (iii) a CAR, a TA, and an AlaDH, or (iv) a CAR, a TA, a PPTase, and an AlaDH.

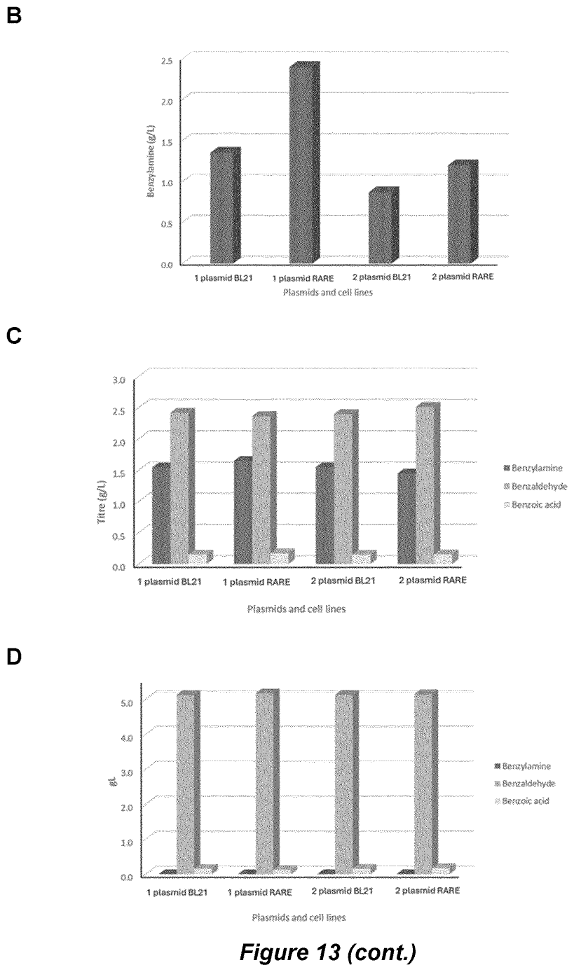

An enzymatic cascade is defined herein as a sequence of successive enzymatic reactions. An enzyme activity may comprise the activity of (i) a CAR and a TA, (ii) a CAR, a TA, and a PPTase, (iii) a CAR, a TA, and an AlaDH, or (iv) a CAR, a TA, a PPTase, and an AlaDH. An exemplary enzymatic cascade is shown in FIG. 13A.

In some embodiments, one plasmid encodes a CAR, TA, PPTase, and/or AlaDH, and another plasmid encodes a CAR, TA, PPTase, and/or AlaDH.

In some embodiments, one plasmid comprises a gene encoding a CAR and a gene encoding a PPTase, and another plasmid comprises a gene encoding a TA and a gene encoding an AlaDH. In some embodiments, a first plasmid comprises a gene encoding a CAR and a gene encoding a PPTase, and a second plasmid comprises a gene encoding a TA and a gene encoding an AlaDH.

In some embodiments, the two-plasmid system comprises a pBbE2c NiCAR_Sfp plasmid and pBbA1k CvTA_BsAlaDH (P3B-CvTA) plasmid. The pBbE2c NiCAR_Sfp plasmid is described in the Examples (e.g., Example 2). The pBbA1k CvTA_BsAlaDH (P3B-CvTA) plasmid is described in the Examples (e.g., Example 2).

In some embodiments, a first plasmid encodes a CAR, TA, PPTase, and/or AlaDH, and a second plasmid encodes a CAR, TA, PPTase, and/or AlaDH.

In some embodiments, a first plasmid comprises nucleic acids encoding a CAR and a PPTase, and a second plasmid comprises nucleic acids encoding a TA and an AlaDH.

In some embodiments, genes are provided in a single-plasmid system. A single-plasmid system is defined herein as a plasmid which comprises the genes necessary for the expression of an enzyme cascade.

In some embodiments, the plasmid comprises nucleic acids encoding a CAR, TA, PPTase, and/or AlaDH. In some embodiments, the plasmid comprises nucleic acids encoding a TA. In some embodiments, the plasmid comprises nucleic acids encoding a CAR. In some embodiments, the plasmid comprises nucleic acids encoding a PPTase. In some embodiments, the plasmid comprises nucleic acids encoding an AlaDH. In some embodiments, the plasmid comprises nucleic acids encoding a CAR, TA, PPTase, and AlaDH.

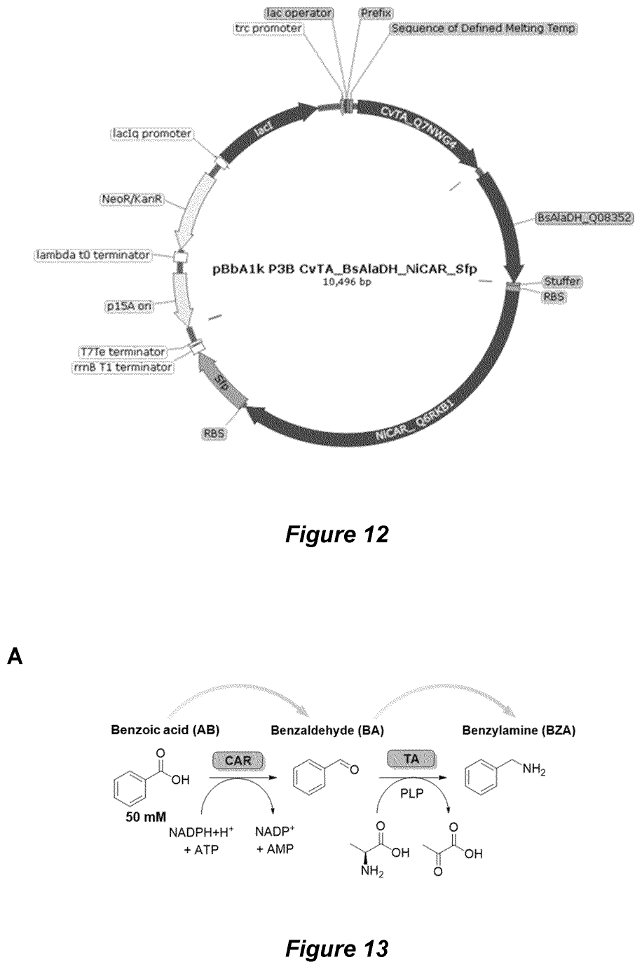

In some embodiments, the plasmid is a pBbA1-P3B-CvTA_BsAla_DH_NiCAR_Sfp (FIG. 12). The pBbA1-P3B-CvTA_BsAla_DH_NiCAR_Sfp plasmid is described in the Examples (e.g., Example 9).Also provided by the present invention is a cell comprising a TA and a CAR, a nucleic acid or plurality of nucleic acids, or an expression vector according to the present invention.

The nucleic acids/expression vectors can be introduced into a cell by any suitable means, which are well known to the skilled person. In some embodiments the nucleic acids/expression vectors are introduced into a cell by transformation, transduction, conjugation, transfection or electroporation.

A cell comprising a TA and/or a CAR according to the present invention may do so through expression from a nucleic acid/expression vector according to the present invention that has been introduced into the cell.

Cells and microorganisms contemplated for use with the present invention include prokaryotic and eukaryotic cells. For example, the prokaryotic cell may be a bacteria or archaea, and the eukaryotic microorganism may be a fungi, protist, or microscopic animal or microscopic plant organism. In some embodiments, the cells are isolated cells from a multicellular organism.

Microorganisms commonly used in commercial and industrial processes are contemplated, including microorganisms used for the commercial or industrial production of chemicals, enzymes or other biological molecules. In preferred aspects, the cells are of a bacterium. In some embodiments, the bacterium may be a Gram-positive bacterium. Gram-positive bacteria include bacteria from the genus Bacillus, bacteria from the genus Listeria, Clostridium (e.g. C. difficile), or cocci such as Staphylococcus (e.g. S. aureus), or Streptococcus. In some embodiments the bacterium may be a Gram-negative bacterium. Gram-negative bacteria may be defined as a class of bacteria that do not retain the crystal violet stain used in the Gram staining method of bacterial differentiation, making positive identification possible. Gram-negative bacteria include proteobacteria or bacteria of the family Enterobacteriaceae, such as Escherichia coli, Salmonella sp, Shigella sp, or bacteria selected from the genus Pseudomonas, Helicobacter, Neisseria, Legionella, Halomonas, Klebsiella or Yersinia bacterium.

In some embodiments, the fungi may be Blastocladiomycota, Chytridiomycota, Glomeromycota, Microsporidia, or Neocallimastigomycota. In some embodiments, the fungi may be Dikarya (including Deuteromycota), such as fungi of the Ascomycota, including Pezizomycotina, Saccharomycotina, and Taphrinomycotina; or Basidiomycota, including Agaricomycotina, Pucciniomycotina, and Ustilaginomycotina. In some embodiments, the fungi may be fungi of the Entomophthoromycotina, Kickxellomycotina, Mucoromycotina, or Zoopagomycotina.

In some embodiments, Escherichia bacteria such as E. coli, Saccharomyces yeast such as S. cerevisiae and cyanobacteria/microalgae are contemplated for use in the present invention. In some embodiments the polypeptides may be prepared by cell-free-protein synthesis (CFPS), e.g. according to a system described in Zemella et al. Chembiochem (2015) 16(17): 2420-2431, which is hereby incorporated by reference in its entirety.

The present invention also provides compositions comprising the cells, nucleic acids, expression vectors, and enzymes/combinations of enzymes according to the present invention. The compositions find use e.g. in methods for amine production according to the present invention.

Any bacterium may be used, such as laboratory strains (such as E. coli or B. subtilis), or field strains. Preferred bacteria will be those that are organotrophic, e.g. chemoheterotrophic bacteria, capable of using biomass or compounds derived therefrom as an energy source.

Preferred bacteria are robust bacteria, such as soil bacteria and/or extremophilic bacteria. Extremophilic bacteria include slight halophiles (able to grow in 1.7 to 4.8% NaCl), moderate halophiles (able to grow in 4.7 to 20% NaCl), extreme halophiles (able to grow in 20 to 30% NaCl), acidophiles (able to grow in conditions of low pH, such as below pH 5.0, e.g. pH 2 or below), alkaliphiles (able to grow in conditions of pH 8.5 or above), metallotolerant bacteria (able to survive in environments containing high concentrations of dissolved heavy metals), thermophiles or hyperthermophiles (with an optimal growth temperature between about 41 and 122° C., e.g. strains of Caldicellulosiruptor, Thermotoga, Thermoanaerobacterium, Pyrococcus, and Aeropyrum), or polyextremophiles (bacterial possessing two or more extremophilic characteristics).

Especially preferred are halophilic bacteria. These are capable of growing in open non-sterile conditions. As these strains are salt tolerant, they will not be outcompeted so long as there is a high enough salt content. Furthermore, the addition of a high salt buffer (e.g. at least a 3% salt solution) can be used to control competing bacteria. Halophilic bacteria include those of the genus Halomonas. Exemplary species of Halomonas have been described, including H. alimentaria, H. alkaliantarctica, H. alkaliphila, H. almeriensis, H. andesensis, H. anticariensis, H. aquamarina, H. arcis, H. axialensis, H. beimenensis, H. bluephagenesis, H. boliviensis, H. campaniensis, H. campisalis, H. caseinilytica, H. cerina, H. cibimaris, H. cupida, H. daqiaonensis, H. daqingensis, H. denitrificans, H. desiderata, H. elongata, H. eurihalina, H. flava, H. fontilapidosi, H. garicola, H. gomseomensis, H. gudaonensis, H. halmophila, H. halocynthiae, H. halodenitrificans, halophila, H. hamiltonii, H. heilongjiangensis, H. huangheensis, H. hydrothermalis, H. ilicicola, H. janggokensis, H. jeotgali, H. johnsoniae, H. kenyensis, H. koreensis, H. korlensis, H. kribbensis, H. lutea, H. lutescence, H. magadiensis, H. maura, H. meridian, H. mongoliensis, H. muralis, H. nanhaiensis, H. neptunia, H. nitroreducens, H. olivaria, H. organivorans, H. pacifica, H. pantelleriensis, H. qiaohouensis, H. qijiaojingensis, H. ramblicola, H. rifensis, H. rowanensis, H. sabkhae, H. saccharevitans, H. salicampi, H. salifodinae, H. salina, H. sediminicola, H. shengliensis, H. sinaiensis, H. smyrnensis, H. songnenensis, H. stenophila, H. stevensii, H. subglaciescola, H. subterranean, H. sulfidaeris, H. taeanensis, H. titanicae, H. urumqiensis, H. variabilis, H. ventosae, H. venusta, H. vilamensis, H. xianhensis, H. xinjiangensis, H. zhangjiangensis, and H. zincidurans.

Preferred Halomonas strains include H. bluephagenesis st. TQ10, H. bluephagenesis st. TD1.0 and H. bluephagenesis st. TD01. Strains TD1.0 and TQ10 are genetically modified versions of the native TD01 strain where the gene encoding MmP1 has been chromosomally integrated into the bacterium. The gene MmP1 is a T7-like promoter that enables the IPTG-inducible expression of recombinant proteins in Halomonas (Zhao H et al 2017 Novel T7-like expression systems used for Halomonas. Metabolic Engineering 39: p. 128-140 which is herein incorporated by reference in its entirety). Preferably, the Halomonas strain comprises the MmP1 gene, either chromosomally integrated or on a vector or plasmid. Strain TQ10 additionally has been genetically modified to knock out gene(s) involved in polyhydroxyalkanoate (PHA) biosynthesis.

### General Definitions

As used herein, a “fragment”, “variant” or “homologue” of a protein may optionally be characterised as having at least 50%, preferably one of 60%, 70%, 75%, 80%, 85%, 90%, 91%, 92%, 93%, 94%, 95%, 96%, 97%, 98%, 99% or 100% amino acid sequence identity to the amino acid sequence of the reference protein. Fragments, variants, isoforms and homologues of a reference protein may be characterised by the ability to perform a function performed by the reference protein.

Pairwise and multiple sequence alignment for the purpose of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

A “fragment” generally refers to a fraction of the reference protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein.

A “fragment” of a reference protein may be of any length (by number of amino acids), although may optionally be at least 25% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

A fragment of a polypeptide may have a minimum length of one of 10, 20, 30, 40, 50, 60, 70, 80, 90 or 100 amino acids, and may have a maximum length of one of 15, 20, 25, 30, 40, 50, 100, 110, 120 or 130 amino acids.

### Sequence Identity

Pairwise and multiple sequence alignment for the purpose of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Nocardia iowensis | MAVDSPDERLQRRIAQLFAEDEQVKAARPLEAVSAAVSAPGMRLAQIAATVMAGYADRPAAGQR |
| | CAR (UniProt: | AFELNTDDATGRTSLRLLPRFETITYRELWQRVGEVAAAWHHDPENPLRAGDFVALLGFTSIDYAT |
| | Q6RKB1) | LDLADIHLGAVTVPLQASAAVSQLIAILTETSPRLLASTPEHLDAAVECLLAGTTPERLVVFDYHPED |
| | | DDQRAAFESARRRLADAGSLVIVETLDAVRARGRDLPAAPLFVPDTDDDPLALLIYTSGSTGTPKG |
| | | AMYTNRLAATMWQGNSMLQGNSQRVGINLNYMPMSHIAGRISLFGVLARGGTAYFAAKSDMSTL |
| | | FEDIGLVRPTEIFFVPRVCDMVFQRYQSELDRRSVAGADLDTLDREVKADLRQNYLGGRFLVAVV |
| | | GSAPLAAEMKTFMESVLDLPLHDGYGSTEAGASVLLDNQIQRPPVLDYKLVDVPELGYFRTDRPH |
| | | PRGELLLKAETTIPGYYKRPEVTAEIFDEDGFYKTGDIVAELEHDRLVYVDRRNNVLKLSQGEFVTV |
| | | AHLEAVFASSPLIRQIFIYGSSERSYLLAVIVPTDDALRGRDTATLKSALAESIQRIAKDANLQPYEIP |
| | | RDFLIETEPFTIANGLLSGIAKLLRPNLKERYGAQLEQMYTDLATGQADELLALRREAADLPVLETVS |
| | | RAAKAMLGVASADMRPDAHFTDLGGDSLSALSFSNLLHEIFGVEVPVGVVVSPANELRDLANYIEA |
| | | ERNSGAKRPTFTSVHGGGSEIRAADLTLDKFIDARTLAAADSIPHAPVPAQTVLLTGANGYLGRFLC |
| | | LEWLERLDKTGGTLICVVRGSDAAAARKRLDSAFDSGDPGLLEHYQQLAARTLEVLAGDIGDPNL |
| | | GLDDATWQRLAETVDLIVHPAALVNHVLPYTQLFGPNVVGTAEIVRLAITARRKPVTYLSTVGVADQ |
| | | VDPAEYQEDSDVREMSAVRVVRESYANGYGNSKWAGEVLLREAHDLCGLPVAVFRSDMILAHSR |
| | | YAGQLNVQDVFTRLILSLVATGIAPYSFYRTDADGNRQRAHYDGLPADFTAAAITALGIQATEGFRT |
| | | YDVLNPYDDGISLDEFVDWLVESGHPIQRITDYSDWFHRFETAIRALPEKQRQASVLPLLDAYRNP |
| | | CPAVRGAILPAKEFQAAVQTAKIGPEQDIPHLSAPLIDKYVSDLELLQLL |
| 2 | Mycobacterium | MASESRDVRLQRRIAELYDTDPQFAAARPDEAVARAVNAPGLTLSQVIRTVLDNYADRPALGYRA |
| | phlei CAR | VEFAADPASARTVARLLPRFDTITYRELGERIDATTAALGHDGLRPGERVAILGFSSVDYTTIDIAAF |
| | (UniProt: | NLGAVSVPLQTSAPPSQLRPMTAETEPAVIAASVDFLDDALELIRTGHAPRRLVVFDFHPEIDDHRD |
| | A0A5N5VEC4) | ALAAATTGLADTAMTVETLDSLLTRGRTLPAPTGYRDRDDDLALLIYTSGSTGAPKGAMYQRRMV |
| | | TNMWRRATTAIWGGKEAAPWLTLNFMPMSHVMGRGILSTTLCSGGTAYFAARSDLSTLLEDLALV |
| | | RPTQLTFVPRIWEMIFQEYQRDVARRPEAEVLADLREHRLGGRFLAAMTGSAPMSPEMTAFAESV |
| | | LDLHLVDGYGSTEAGSIVLDGQVLRPPVLDYKLVDVPELGYFSTDRPYPRGELLVKTELMFPGYYK |
| | | RPDITAEVFDEDGYYRTGDIVAELGPDRLAYVDRRNNVLKLSQGEFVTVSKLEAAFAASPLVRQIYI |
| | | YGNSAHPYLLAVVVPTEDALTRYDAATLKTAISESLQDVGRAAGLQSYEIPRDFLVETTPFTLENGL |
| | | LTGLRKLARPKLKEHYGERLEQLYTELADGQAEELKELRTHGAQQPTLTTVSRAATALLGTASAEV |
| | | RPDAHFTDLGGDSLSALTFGNLLGEIYAVEVPVGVIVSPANDLAAIADYIDTARRPGDGRPTFAGVH |
| | | GDDAAEVHARDLTLDRFLDDATLAAATALPGPAPEIRTVLLTGATGFLGRYLALEWLERMAMVGG |
| | | TLICLVRGRDDAAARARLDQIFDSGDPELLRHYRELADRHLEVIAGDKSDAVLGLDRRTWQRLADT |
| | | VDLIVDPAALVNHVLPYRELFGPNVVGTAELIRLALTGRLKPYLYTSTIAVGAGIAPGQFTEDADIRQI |
| | | SATRTLDDSYANGYATSKWAGEVLLREAHDLCGLPVAVFRCDMILADTSYAGQLNVPDMFTRLILS |
| | | LVATGIAPLSFYELDAAGHRQRAHYDGLPVEFVAEAVSALGLDVAEDGGFATYHVMNPYDDGIGL |
| | | DEFVDWLTGAGYPIERVNDYGTWFQRFETAIRGLPERQRQASLLPLLHNYQRPQPPIRGSAAPTD |
| | | RFRSAVQDAKIGPDKDIPHITPEVIVKYVTDLRLLGLL |
| 3 | Mycobacterium | MTIETREDRFNRRIDHLFETDPQFAAARPDEAISAAAADPELRLPAAVKQILAGYADRPALGKRAVE |
| | smegmatis CAR1 | FVTDEEGRTTAKLLPRFDTITYRQLAGRIQAVTNAWHNHPVNAGDRVAILGFTSVDYTTIDIALLELG |
| | (UniProt: | AVSVPLQTSAPVAQLQPIVAETEPKVIASSVDFLADAVALVESGPAPSRLVVFDYSHEVDDQREAF |
| | A0A653FAQ0) | EAAKGKLAGTGVVVETITDALDRGRSLADAPLYVPDEADPLTLLIYTSGSTGTPKGAMYPESKTAT |
| | | MWQAGSKARWDETLGVMPSITLNFMPMSHVMGRGILCSTLASGGTAYFAARSDLSTFLEDLALV |
| | | RPTQLNFVPRIWDMLFQEYQSRLDNRRAEGSEDRAEAAVLEEVRTQLLGGRFVSALTGSAPISAE |
| | | MKSWVEDLLDMHLLEGYGSTEAGAVFIDGQIQRPPVIDYKLVDVPDLGYFATDRPYPRGELLVKSE |
| | | QMFPGYYKRPEITAEMFDEDGYYRTGDIVAELGPDHLEYLDRRNNVLKLSQGEFVTVSKLEAVFG |
| | | DSPLVRQIYVYGNSARSYLLAVVVPTEEALSRWDGDELKSRISDSLQDAARAAGLQSYEIPRDFLV |
| | | ETTPFTLENGLLTGIRKLARPKLKAHYGERLEQLYTDLAEGQANELRELRRNGADRPVVETVSRAA |
| | | VALLGASVTDLRSDAHFTDLGGDSLSALSFSNLLHEIFDVDVPVGVIVSPATDLAGVAAYIEGELRG |
| | | SKRPTYASVHGRDATEVRARDLALGKFIDAKTLSAAPGLPRSGTEIRTVLLTGATGFLGRYLALEW |
| | | LERMDLVDGKVICLVRARSDDEARARLDATFDTGDATLLEHYRALAADHLEVIAGDKGEADLGLDH |
| | | DTWQRLADTVDLIVDPAALVNHVLPYSQMFGPNALGTAELIRIALTTTIKPYVYVSTIGVGQGISPEA |
| | | FVEDADIREISATRRVDDSYANGYGNSKWAGEVLLREAHDWCGLPVSVFRCDMILADTTYSGQLN |
| | | LPDMFTRLMLSLVATGIAPGSFYELDADGNRQRAHYDGLPVEFIAEAISTIGSQVTDGFETFHVMN |
| | | PYDDGIGLDEYVDWLIEAGYPVHRVDDYATWLSRFETALRALPERQRQASLLPLLHNYQQPSPPV |
| | | CGAMAPTDRFRAAVQDAKIGPDKDIPHVTADVIVKYISNLQMLGLL |
| 4 | Mycobacterium | MTSDVHDATDGVTETALDDEQSTRRIAELYATDPEFAAAAPLPAVVDAAHKPGLRLAEILQTLFTG |
| | smegmatis CAR2 | YGDRPALGYRARELATDEGGRTVTRLLPRFDTLTYAQVWSRVQAVAAALRHNFAQPIYPGDAVAT |
| | (UniProt: | IGFASPDYLTLDLVCAYLGLVSVPLQHNAPVSRLAPILAEVEPRILTVSAEYLDLAVESVRDVNSVSQ |
| | A0A653FCI4) | LVVFDHHPEVDDHRDALARAREQLAGKGIAVTTLDAIADEGAGLPAEPIYTADHDQRLAMILYTSGS |
| | | TGAPKGAMYTEAMVARLWTMSFITGDPTPVINVNFMPLNHLGGRIPISTAVQNGGTSYFVPESDM |
| | | STLFEDLALVRPTELGLVPRVADMLYQHHLATVDRLVTQGADELTAEKQAGAELREQVLGGRVITG |
| | | FVSTAPLAAEMRAFLDITLGAHIVDGYGLTETGAVTRDGVIVRPPVIDYKLIDVPELGYFSTDKPYPR |
| | | GELLVRSQTLTPGYYKRPEVTASVFDRDGYYHTGDVMAETAPDHLVYVDRRNNVLKLAQGEFVA |
| | | VANLEAVESGAALVRQIFVYGNSERSFLLAVVVPTPEALEQYDPAALKAALADSLQRTARDAELQS |
| | | YEVPADFIVETEPFSAANGLLSGVGKLLRPNLKDRYGQRLEQMYADIAATQANQLRELRRAAATQP |
| | | VIDTLTQAAATILGTGSEVASDAHFTDLGGDSLSALTLSNLLSDFFGFEVPVGTIVNPATNLAQLAQ |
| | | HIEAQRTAGDRRPSFTTVHGADATEIRASELTLDKFIDAETLRAAPGLPKVTTEPRTVLLSGANGWL |
| | | GRFLTLQWLERLAPVGGTLITIVRGRDDAAARARLTQAYDTDPELSRRFAELADRHLRVVAGDIGD |
| | | PNLGLTPEIWHRLAAEVDLVVHPAALVNHVLPYRQLFGPNVVGTAEVIKLALTERIKPVTYLSTVSV |
| | | AMGIPDFEEDGDIRTVSPVRPLDGGYANGYGNSKWAGEVLLREAHDLCGLPVATFRSDMILAHPR |
| | | YRGQVNVPDMFTRLLLSLLITGVAPRSFYIGDGERPRAHYPGLTVDFVAEAVTTLGAQQREGYVSY |
| | | DVMNPHDDGISLDVFVDWLIRAGHPIDRVDDYDDWVRRFETALTALPEKRRAQTVLPLLHAFRAP |
| | | QAPLRGAPEPTEVFHAAVRTAKVGPGDIPHLDEALIDKYIRDLREFGLI |
| 5 | Nocardia | MLDDARAERRERRIADALADDQVREAVADAAVSESVRRVEVRLARIVDAVMSGYGDRAALAWRR |
| | otitidiscaviarum | SELVDGAVRLLPEYSTMTYRELWRRAGAVAAEWGADAESPVRAGDFVCTLGFTSPDYTVVDLAL |
| | CAR (UniProt: | VRLGAVAVPLQASASVAQWRSIMAETEPRMLATSAETLPAAVEAVLGGFAPRRVLVFDYRPELEA |
| | A0A516NPD0) | HRSAVDSARERLAEVGCRVATVADAVDRGASLPAPLCIPSDRERLALLIYTSGSTGAPKGAMYTD |
| | | RLAAGLWLSANDIRVPALTMNYMPLSHVAGRMSLYGTLMRGGTAYFAAASDMSTLLDDFGLARPT |
| | | ELFLVPRVCELLHQRYQSELDRRVLAGDDVETTATDVKAELRERVLGGRYLTALSGSAPLAAEMK |
| | | TFMESLLDDELHDGYGSTEAGGSVLLDNRIKRPPVLDYRLVDVPELGYFRTDKPHPRGELLLKTES |
| | | MFPGYYKRPEITAEMFDADGFYRTGDVVAELGPDQLVYVDRRNNVLKLSQGEFVTVAALEAVYAT |
| | | SPLIRQIFVYGSSERAYLLAVVVPTDEVLALPPARARAEVGESLHRIAKESGLQPYEIPREVLIESEP |
| | | FTIDNGLLSGIGKLLRPKLKEHYGEQLERLYTELAQQREDELTTLRRGAHERPVLETVTRAAGAVLD |
| | | LAAGEVSPDAHFADLGGDSLSALSFSTLLRDIFGVEVPVGVIVGPATDLTRIAEYLVAERDSGTRPT |
| | | AATVHGDDGLLRADDLTLEAFLDPGTLAAATRLSAPTDPPRTVLLTGANGYLGRFLALEWLQRLDV |
| | | SGGTLICLVRGSDADAARRRLDAVFATGDPELEAHYRDLAGRRLRVLAGDIGEPNLGLSEQDWRD |
| | | LAESVDVIVHPAALVNHVLPYAQLFGPNVVGTAEVIRLALTARLKPVTYLSTVAVSAGIDPETFTEDG |
| | | DIREISPVRRLDDGYANGY GNSKWAGEVLLRAAHDRFGLPVAVFRSDMILAHSHYAGQLNVPDMF |
| | | TRLLLSVLATGLAPGSFHDAHGERHRAHYDGLPADFTAAAVTTLGAQATSGFDTYDVLNPHDDGL |
| | | SLDTFIDWLIDSGHPIDRIDDYADWFTRFETAVRALPEPQRQHSLLPLLHAYRHPTPPLRGAALPAK |
| | | HFRAAVQQAKLGPDADIPHLTRELIEKYVSDLRLLGLLG |
| 6 | Tsukamurella | MSIETVQNGVPAEGSVPPADQQTERLPQVIARIFAQFADRPAFATREAGPGTPYATVSYREIWRRV |
| | paurometabola | TALVASWQSEVAPGDFVAILGFTSSDFVTVDLATTLLGAPNVPLQAGAPAARIATILDETRPKILAVS |
| | CAR (UniProt: | ADQVDLAQEALAESAATPRVVVFDGERDGYEGIEADILSGSALPAPEFFAPEPGTDPLVTLIYTSGS |
| | D5UWX9) | TGTPKGAMYTEQLVRDAWLKVDSIVDIDMPAESLLHFLPMSHMYGRNWLIAGLASGGTGYFAGAS |
| | | DMSTLFDDLAAARPTAIGLVPRVCELIHQRYLAVEADTDAETARVELRDRVLGGRLQAAMCGSAAL |
| | | SSELQTFMEWLLGIDIQIGYGSTEAGGVIRDGVVVRPPVTEYKLIDVPELGYFVTDSPHPRGELLVK |
| | | STQLIPGYYNSDKRIRDDEGFYRTGDVMAELGPDRLEYVDRRSNVIKLAQGEFVPIAQLEAIYAAGP |
| | | DVHQIFLYGTSERSYLIGVVVPAPGPDGETDAQTRTRVLDGLAAIARENDLAAYEVPRDVLIERDPF |
| | | SQENGLRSGIGKLVRPALIARYGDRLHDLYAQADTRQREGLRALDASGPIIDTVLGAAALTLGADIA |
| | | DFDADTRFGDLGGDSLSALSLATTLEGLYDVPVPVQTIVGPTATLGGVARHIEKARSGGVAAPTAD |
| | | SVHGVGASVARATDLTLEKFIDPELLALAPTLPAATGEPNTVLLTGSTGYLGRFLLLDWLRRVAPH |
| | | GGTVIALVRGADADDARRRVTAAIGDSDPDLTQEFTSLAEHHLHVIAGDFGSPALGLDDATWSDLA |
| | | GRVDHVVHCGALVNHVLPYDQLFGPNVVATGEVVRLALTTRRKSVDYVSTVAVVPQDDGRVLVE |
| | | DDDVRELGAERRIGADAYANGYAVSKWAGEVLLHEAADLADLPVRVFRSDMILAHSRFHGQFNEV |
| | | DQFTRLLLSIAETGLAPASFYTPDPSGHRPHYDGLPVDFTAEAITTLSAAGRSGYRTFHVLNANDD |
| | | GVSLDSFVDWIAASGRSIERIDDYDTWFARFEQALQQLPDEARQRSVLPLLHAVREPAPAAGTSAL |
| | | SVDRFRGAVRETGVGPGDIPVLDRALIEKYLRDFETAGWLAPGARD |
| 7 | Chromobacterium | MQKQRTTSQWRELDAAHHLHPFTDTASLNQAGARVMTRGEGVYLWDSEGNKIIDGMAGLWCVN |
| | violaceum TA | VGYGRKDFAEAARRQMEELPFYNTFFKTTHPAVVELSSLLAEVTPAGFDRVFYTNSGSESVDTMI |
| | (UniProt: | RMVRRYWDVQGKPEKKTLIGRWNGYHGSTIGGASLGGMKYMHEQGDLPIPGMAHIEQPWWYKH |
| | Q7NWG4) | GKDMTPDEFGVVAARWLEEKILEIGADKVAAFVGEPIQGAGGVIVPPATYWPEIERICRKYDVLLVA |
| | | DEVICGFGRTGEWFGHQHFGFQPDLFTAAKGLSSGYLP\|GAVFVGKRVAEGLIAGGDFNHGFTYS |
| | | GHPVCAAVAHANVAALRDEGIVQRVKDDIGPYMQKRWRETFSRFEHVDDVRGVGMVQAFTLVKN |
| | | KAKRELFPDFGEIGTLCRDIFFRNNLIMRACGDHIVSAPPLVMTRAEVDEMLAVAERCLEEFEQTLK |
| | | ARGLA |
| 8 | Vibrio fluvialis TA | MNKPQSWEARAETYSLYGFTDMPSLHQRGTVVVTHGEGPYIVDVNGRRYLDANSGLWNMVAGF |
| | (UniProt: F2XBU9) | DHKGLIDAAKAQYERFPGYHAFFGRMSDQTVMLSEKLVEVSPFDSGRVFYTNSGSEANDTMVKM |
| | | LWFLHAAEGKPQKRKILTRWNAYHGVTAVSASMTGKPYNSVFGLPLPGFVHLTCPHYWRYGEEG |
| | | ETEEQFVARLARELEETIQREGADTIAGFFAEPVMGAGGVIPPAKGYFQAILPILRKYDIPVISDEVIC |
| | | GFGRTGNTWGCVTYDFTPDAIISSKNLTAGFFPMGAVILGPELSKRLETAIEAIEEFPHGFTASGHP |
| | | VGCAIALKAIDVVMNEGLAENVRRLAPRFEERLKHIAERPNIGEYRGIGFMWALEAVKDKASKTPFD |
| | | GNLSVSERIANTCTDLGLICRPLGQSVVLCPPFILTEAQMDEMFDKLEKALDKVFAEVA |
| 9 | Alcaligenes | MSAAKLPDLSHLWMPFTANRQFKANPRLLASAKGMYYTSFDGRQILDGTAGLWCVNAGHCREEI |
| | denitrificans TA | VSAIASQAGVMDYAPGFQLGHPLAFEAATAVAGLMPQGLDRVFFTNSGSESVDTALKIALAYHRA |
| | (UniProt: | RGEAQRTRLIGRERGYHGVGFGGISVGGISPNRKTFSGALLPAVDHLPHTHSLEHNAFTRGQPEW |
| | Q7WWK8) | GAHLADELERIIALHDASTIAAVIVEPMAGSTGVLVPPKGYLEKLREITARHGILLIFDEVITAYGRLGE |
| | | ATAAAYFGVTPDLITMAKGVSNAAVPAGAVAVRREVHDAIVNGPQGGIEFFHGYTYSAHPLAAAAV |
| | | LATLDIYRREDLFARARKLSAAFEEAAHSLKGAPHVIDVRNIGLVAGIELSPREGAPGARAAEAFQK |
| | | CFDTGLMVRYTGDILAVSPPLIVDENQIGQIFEGIGKVLKEVA |
| 10 | Pseudomonas | MSEQNSQTLAWQTMSRDHHLAPFSDVRQLAEKGPRIITSAKGVYLWDSEGNKILDGMAGLWCVA |
| | putida TA (UniProt: | VGYGREELAEVASQQMKQLPYYNLFFQTAHPPALELAKAIAEVAPQGMNHVFFTGSGSEGNDTVL |
| | Q88KV9) | RMVRHYWALKGQKNKKVIIGRINGYHGSTVAGAALGGMSGMHQQGGVIPDVVHIPQPYWFGEGG |
| | | DMTEADFGVWAAEQLEKKILEVGVDNVAAFIAEPIQGAGGVIIPPQTYWPKIKEILARYDILFVADEVI |
| | | CGFGRTGEWFGTDYYDLKPDLMTIAKGLTSGYIPMGGVIVRDEVAKVISEGGDFNHGFTYSGHPV |
| | | AAAVGLENLRILRDEQIIQQVHDKTAPYLQQRLRELADHPLVGEVRGLGMLGAIELVKDKATRARH |
| | | EGKGVGMICRQHCFDNGLIMRAVGDTMIIAPPLVISIEEIDELVEKARKCLDLTYEAVR |
| 11 | Paracoccus | MNQPQSWEARAETYSLYGFTDMPSVHQRGTVVVTHGEGPYIVDVHGRRYLDANSGLWNMVAGF |
| | denitrificans TA | DHKGLIEAAKAQYDRFPGYHAFFGRMSDQTVMLSEKLVEVSPFDNGRVFYTNSGSEANDTMVKM |
| | (UniProt: A1B956) | LWFLHAAEGKPQKRKILTRWNAYHGVTAVSASMTGKPYNSVFGLPLPGFIHLTCPHYWRYGEEG |
| | | ETEAQFVARLARELEDTITREGADTIAGFFAEPVMGAGGVIPPAKGYFQAILPILRKYDIPMISDEVIC |
| | | GFGRTGNTWGCLTYDFMPDAIISSKNLTAGFFPMGAVILGPDLAKRVEAAVEAIEEFPHGFTASGH |
| | | PVGCAIALKAIDVVMNEGLAENVRRLAPRFEAGLKRIADRPNIGEYRGIGFMWALEAVKDKPTKTPF |
| | | DANLSVSERIANTCTDLGLICRPLGQSIVLCPPFILTEAQMDEMFEKLEKALDKVFAEVA |
| 12 | Bacillus subtilis Sfp | MKIYGIYMDRPLSQEENERFMSFISPEKREKCRRFYHKEDAHRTLLGDVLVRSVISRQYQLDKSDI |
| | (Uniprot: P39135) | RFSTQEYGKPCIPDLPDAHFNISHSGRWVICAFDSQPIGIDIEKTKPISLEIAKRFFSKTEYSDLLAKD |
| | | KDEQTDYFYHLWSMKESFIKQEGKGLSLPLDSFSVRLHQDGQVSIELPDSHSPCYIKTYEVDPGYK |
| | | MAVCAAHPDFPEDITMVSYEELL |
| 13 | Bacillus subtilis | MIIGVPKEIKNNENRVALTPGGVSQLISNGHRVLVETGAGLGSGFENEAYESAGAEIIADPKQVWD |
| | AlaDH (Uniprot: | AEMVMKVKEPLPEEYVYFRKGLVLFTYLHLAAEPELAQALKDKGVTAIAYETVSEGRTLPLLTPMS |
| | Q08352) | EVAGRMAAQIGAQFLEKPKGGKGILLAGVPGVSRGKVTIIGGGVVGTNAAKMAVGLGADVTIIDLN |
| | | ADRLRQLDDIFGHQIKTLISNPVNIADAVAEADLLICAVLIPGAKAPTLVTEEMVKQMKPGSVIVDVAI |
| | | DQGGIVETVDHITTHDQPTYEKHGVVHYAVANMPGAVPRTSTIALTNVTVPYALQIANKGAVKALA |
| | | DNTALRAGLNTANGHVTYEAVARDLGYEYVPAEKALQDESSVAGA |
| 14 | E. coli 2,5-diketo- | MANPTVIKLQDGNVMPQLGLGVWQASNEEVITAIQKALEVGYRSIDTAAAYKNEEGVGKALKNASV |
| | D-gluconic acid | NREELFITTKLWNDDHKRPREALLDSLKKLQLDYIDLYLMHWPVPAIDHYVEAWKGMIELQKEGLIK |
| | reductase A | SIGVCNFQIHHLQRLIDETGVTPVINQIELHPLMQQRQLHAWNATHKIQTESWSPLAQGGKGVFDQ |
| | (UniProt: Q46857) | KVIRDLADKYGKTPAQIVIRWHLDSGLVVIPKSVTPSRIAENFDVWDFRLDKDELGEIAKLDQGKRL |
| | | GPDPDQFGG |
| 15 | E. coli 2,5-diketo- | MAIPAFGLGTFRLKDDVVISSVITALELGYRAIDTAQIYDNEAAVGQAIAESGVPRHELYITTKIWIENL |
| | D-gluconic acid | SKDKLIPSLKESLQKLRTDYVDLTLIHWPSPNDEVSVEEFMQALLEAKKQGLTREIGISNFTIPLMEK |
| | reductase B | AIAAVGAENIATNQIELSPYLQNRKVVAWAKQHGIHITSYMTLAYGKALKDEVIARIAAKHNATPAQV |
| | (Uniprot: P30863) | ILAWAMGEGYSVIPSSTKRKNLESNLKAQNLQLDAEDKKAIAALDCNDRLVSPEGLAPEWD |
| 16 | E. coli yeaE | MQQKMIQFSGDVSLPAIGQGTWYMGEDASQRKTEVAALRAGIELGLTLIDTAEMYADGGAEKVVG |
| | (UniProt: | EALTGLREKVFLVSKVYPWNAGGQKAINACEASLRRLNTDYLDLYLLHWSGSFALEETVAAMEKL |
| | A0A0Q2YWH0) | VAQGKIRRWGVSNLDYADMQELWQLPGGNQCATNQVLYHLGSRGIEYDLLPWCQQQQIPVMAY |
| | | SPLAQAGRLRNGLLKNAVVNEIAHAHNISSAQVLLAWVISHQGVMAIPKAATIAHVQQNAAVLEVEL |
| | | SPAELAMLDKAYPAPKGKTALDMV |
| 17 | E. coli Aldehyde | MKIKAVGAYSAKQPLEPMDITRREPGPNDVKIEIAYCGVCHSDLHQVRSEWAGTVYPCVPGHEIV |
| | reductase (YAHK, | GRVVAVGDQVEKYAPGDLVGVGCIVDSCKHCEECEDGLENYCDHMTGTYNSPTPDEPGHTLGG |
| | YahK) (UniProt: | YSQQIVVHERYVLRIRHPQEQLAAVAPLLCAGITTYSPLRHWQAGPGKKVGVVGIGGLGHMGIKLA |
| | P75691) | HAMGAHVVAFTTSEAKREAAKALGADEVVNSRNADEMAAHLKSFDFILNTVAAPHNLDDFTTLLKR |
| | | DGTMTLVGAPATPHKSPEVFNLIMKRRAIAGSMIGGIPETQEMLDFCAEHGIVADIEMIRADQINEA |
| | | YERMLRGDVKYRFVIDNRTLTD |
| 18 | E. coli yqhD | MNNFNLHTPTRILFGKGAIAGLREQIPHDARVLITYGGGSVKKTGVLDQVLDALKGMDVLEFGGIEP |
| | (UniProt: Q46856) | NPAYETLMNAVKLVREQKVTFLLAVGGGSVLDGTKFIAAAANYPENIDPWHILQTGGKEIKSAIPMG |
| | | CVLTLPATGSESNAGAVISRKTTGDKQAFHSAHVQPVFAVLDPVYTYTLPPRQVANGVVDAFVHT |
| | | VEQYVTKPVDAKIQDRFAEGILLTLIEDGPKALKEPENYDVRANVMWAATQALNGLIGAGVPQDWA |
| | | THMLGHELTAMHGLDHAQTLAIVLPALWNEKRDTKRAKLLQYAERVWNITEGSDDERIDAAIAATR |
| | | NFFEQLGVPTHLSDYGLDGSSIPALLKKLEEHGMTQLGENHDITLDVSRRIYEAAR |
| 19 | Aldehyde | MSMIKSYAAKEAGGELEVYEYDPGELRPQDVEVQVDYCGICHSDLSMIDNEWGFSQYPLVAGHE |
| | reductase (AHR, | VIGRVVALGSAAQDKGLQVGQRVGIGWTARSCGHCDACISGNQINCEQGAVPTIMNRGGFAEKL |
| | Ahr, YJGB, yjgB) | RADWQWVIPLPENIDIESAGPLLCGGITVFKPLLMHHITATSRVGVIGIGGLGHIAIKLLHAMGCEVT |
| | (UniProt: P27250) | AFSSNPAKEQEVLAMGADKVVNSRDPQALKALAGQFDLIINTVNVSLDWQPYFEALTYGGNFHTV |
| | | GAVLTPLSVPAFTLIAGDRSVSGSATGTPYELRKLMRFAARSKVAPTTELFPMSKINDAIQHVRDGK |
| | | ARYRVVLKADF |
| 20 | Arabidopsis | MDQIEAMLCGGGEKTKVAVTTKTLADPLNWGLAADQMKGSHLDEVKKMVEEYRRPVVNLGGETL |
| | thaliana | TIGQVAAISTVGGSVKVELAETSRAGVKASSDWVMESMNKGTDSYGVTTGFGATSHRRTKNGTA |
| | Phenylalanine | LQTELIRFLNAGIFGNTKETCHTLPQSATRAAMLVRVNTLLQGYSGIRFEILEAITSLLNHNISPSLPL |
| | ammonia-lyase 2 | RGTITASGDLVPLSYIAGLLTGRPNSKATGPDGESLTAKEAFEKAGISTGFFDLQPKEGLALVNGTA |
| | (PAL2; AtPAL2) | VGSGMASMVLFEANVQAVLAEVLSAIFAEVMSGKPEFTDHLTHRLKHHPGQIEAAAIMEHILDGSS |
| | (UniProt: P45724) | YMKLAQKVHEMDPLQKPKQDRYALRTSPQWLGPQIEVIRQATKSIEREINSVNDNPLIDVSRNKAIH |
| | | GGNFQGTPIGVSMDNTRLAIAAIGKLMFAQFSELVNDFYNNGLPSNLTASSNPSLDYGFKGAEIAM |
| | | ASYCSELQYLANPVTSHVQSAEQHNQDVNSLGLISSRKTSEAVDILKLMSTTFLVGICQAVDLRHLE |
| | | ENLRQTVKNTVSQVAKKVLTTGINGELHPSRFCEKDLLKVVDREQVFTYVDDPCSATYPLMQRLR |
| | | QVIVDHALSNGETEKNAVTSIFQKIGAFEEELKAVLPKEVEAARAAYGNGTAPIPNRIKECRSYPLY |
| | | RFVREELGTKLLTGEKVVSPGEEFDKVFTAMCEGKLIDPLMDCLKEWNGAPIPIC |
| 21 | Glycine max 4- | MTTVAASLDAKEVSTSKTDQNQVCDCDPQTSHVFKSKLPDIPISNHLPLHAYCFQKLSQFSDRPCL |
| | coumarate:CoA | IVGPAAKTYTYSETHLISRKIAAGLSNLGIRKGDVVMILLQNSAEFVFSFLAASMIGAVATTANPFYTA |
| | ligase (4CL; | AEIFKQFTVSKTKLIITQAMYVDKLRNHDDGAKLGEDFKVVTVDDPPENCLHFSVLSEANESDAPEV |
| | Gm4CL) (UniProt: | DIQPDDAVAMPFSSGTTGLPKGVVLTHKSLTTSVAQQVDGENPNLYLTTEDVLLCVLPLFHIFSLNS |
| | Q8S5C2) | VLLCALRAGSAVLLMQKFEIGTLLELIQRHRVSVAMVVPPLVLALAKNPMVADFDLSSIRLVLSGAA |
| | | PLGKELVEALRNRVPQAVLGQGYGMTEAGPVLSMCLGFAKQPFPTKSGSCGTVVRNAELRVVDP |
| | | ETGRSLGYNQPGEICIRGQQIMKGYLNDEKATALTIDSEGWLHTGDVGYVDEDDEIFIVDRVKELIK |
| | | YKGFQVPPAELEGLLVSHPSIADAAVVPQKDVAAGEVPVAFVVRSNGFDLTEEAVKEFIAKQVVFY |
| | | KRLHKVYFVHAIPKSPSGKILRKDLRAKLETAATQTP |
| 22 | phdE from | MTTSTTPNTIISFEDAPTLTGQDLGFSQWRTVTQEMVNTFADATDDQQWIHTDPERAKDGPFGGA |
| | Corynebacterium | IAHGFLTLSMIIPFWGELLDVTGVTTKVNYGLDKVRFTSPVKVGSRIRMGAVVREISEVKGNGLHLV |
| | glutamicum | ADGTIEIEGQERPAVVATFLTRFYA |
| | (GENBANK: | |
| | AGN20978.1) | |
| 23 | phdB from | MSLNGKVAIVTGSGAGLGRSFAQELARQGASVIVNDVNQAAADETVAAITEAGGKAAAVIAPVGPS |
| | Corynebacterium | ESAALLVREAVDKFGSLDILVTNAGILRDRSLLKMTDDDFDAVINVHLKGTFTCVRETFGYFKENGI |
| | glutamicum | AGRIVTIGSPTGQRGNFGQSNYAAAKAGIVGMVRTWALEMKRAGVTINAIIPEAATDMTKTVPYFQ |
| | (GENBANK: | KAVEADERGEAMPAFFRETLGFGTPQDVAGLVAFLSSDEAANISGQAIGAGGDRMQVWKHPEPA |
| | AGN20975.1) | VTEFNPGGWTYEALQERGKNIIEDNLQSVGVVFPELPAELQPQIPVKA |
| 24 | phdC from | MSNNVVKYECAVDADNIVAVDMHVHLEVDSCGHESMPADIMEASSKYFKAAERTPSADAIADIYR |
| | Corynebacterium | EHKMAAVVFTIDARTQMGHLPNSIDDLVASCARNNDVLIPFGSVDPRTGEDALVEARRQVEELGV |
| | glutamicum | RGFKFHPSVQGFDPSAPEFYPLWELLESFGLPCVFHTGQNGMGAGLPGGRGIKLRFSNPLLLDDI |
| | (GENBANK: | AADFPNLTIIMAHPSVPWQDEANSIATHKANVFIDLSGWSPKYFPESLVRQSNNVLSKKVLFGTDF |
| | AGN20976.1) | PLITPEKWLAAFANLPLKDEVRPGILKDNAVKVLGLAVNTERGSQAEKAVQHA |
| 25 | PaTA UNIPROT: | MNSQITNAKTREWQALSRDHHLPPFTDYKQLNEKGARIITKAEGVYIWDSEGNKILDAMAGLWCV |
| | Q916J2 amino acid | NVGYGREELVQAATRQMRELPFYNLFFQTAHPPVVELAKAIADVAPEGMNHVFFTGSGSEANDTV |
| | sequence | LRMVRHYWATKGQPQKKVVIGRWNGYHGSTVAGVSLGGMKALHEQGDFPIPGIVHIAQPYWYGE |
| | | GGDMSPDEFGVWAAEQLEKKILEVGEENVAAFIAEPIQGAGGVIVPPDTYWPKIREILAKYDILFIAD |
| | | EVICGFGRTGEWFGSQYYGNAPDLMPIAKGLTSGYIPMGGVVVRDEIVEVLNQGGEFYHGFTYSG |
| | | HPVAAAVALENIRILREEKIIEKVKAETAPYLQKRWQELADHPLVGEARGVGMVAALELVKNKKTRE |
| | | RFTDKGVGMLCREHCFRNGLIMRAVGDTMIISPPLVIDPSQIDELITLARKCLDQTAAAVLA |
| 26 | pBbA1-P3B- | gaattcaaaagatctgagtcttgtaactcccgtattcattttgaacatcgggacaatcc |
| | CVTA_BsAla_DH_ | aatatatgtacggttaagtggtatccgcagtcggat |
| | NICAR_Sfp | acggcaaaggaggatttcaaATGCAGAAACAGCGGACTACCAGCCAGTGGCGCGAACTTGATGCCGC |
| | | CCATCATCTACATCCGTTTACCGACACCGCCTCCCTGAATCAAGCGGGAGCGAGGGTGATGA |
| | | CCCGCGGCGAAGGCGTATACTTATGGGATTCTGAAGGGAACAAAATTATCGATGGCATGGCG |
| | | GGCCTGTGGTGCGTGAATGTGGGTTACGGCCGCAAGGACTTCGCGGAGGCGGCCCGCCGC |
| | | CAGATGGAAGAATTGCCGTTTTACAACACGTTTTTCAAGACCACCCATCCTGCAGTGGTCGAG |
| | | CTCAGCTCCTTACTGGCGGAAGTCACCCCTGCAGGTTTTGATCGTGTTTTCTATACAAACTCC |
| | | GGCAGTGAATCCGTGGATACCATGATCCGCATGGTACGCCGCTATTGGGATGTGCAAGGGAA |
| | | ACCGGAAAAGAAAACGCTGATTGGCCGATGGAACGGCTACCATGGCAGCACCATTGGTGGTG |
| | | CGTCATTGGGTGGGATGAAATATATGCACGAGCAGGGCGATCTGCCAATTCCGGGAATGGCC |
| | | CACATAGAGCAGCCTTGGTGGTATAAACATGGTAAAGACATGACCCCGGACGAGTTCGGCGT |
| | | GGTGGCCGCCCGTTGGCTGGAAGAGAAAATCCTCGAAATCGGAGCGGACAAGGTGGCGGCC |
| | | TTTGTTGGTGAGCCCATTCAGGGCGCTGGAGGTGTTATTGTCCCGCCCGCCACTTATTGGCC |
| | | GGAAATTGAACGTATTTGCCGAAAATATGATGTCTTACTGGTGGCCGATGAAGTGATATGCGG |
| | | CTTCGGCCGTACAGGCGAATGGTTCGGTCATCAACATTTCGGTTTTCAGCCGGATTTGTTCAC |
| | | GGCGGCCAAAGGTTTGTCCTCAGGCTATCTGCCGATAGGAGCAGTATTTGTTGGTAAACGCG |
| | | TGGCCGAAGGGCTGATTGCGGGCGGCGATTTCAACCATGGTTTTACCTACAGCGGTCATCCG |
| | | GTCTGCGCCGCAGTGGCGCATGCGAACGTTGCTGCACTTCGCGACGAGGGCATTGTTCAGC |
| | | GTGTGAAGGATGATATTGGCCCGTATATGCAAAAACGATGGCGCGAAACATTTAGCCGTTTTG |
| | | AACACGTTGATGACGTACGCGGCGTTGGCATGGTGCAGGCCTTTACCCTTGTGAAGAACAAA |
| | | GCGAAACGCGAGCTGTTTCCGGATTTTGGTGAAATTGGCACGTTGTGTCGTGATATTTTCTTTC |
| | | GTAACAATCTGATAATGCGTGCCTGCGGGGATCACATAGTATCGGCACCGCCGCTGGTGATG |
| | | ACGCGCGCCGAGGTCGATGAGATGTTGGCCGTGGCCGAGCGCTGTCTTGAAGAATTTGAACA |
| | | AACCTTGAAAGCGCGTGGACTAGCATAAtctatatgtgacggtaataggcgtca |
| | | cggtcagtcaatcggttctcatgtagcagatccgagaaagcggattcaagt |
| | | tagtgaggtcgaATGATAATTGGCGTTCCAAAGGAAATTAAAAATAACG |
| | | AGAACCGCGTCGCACTGACCCCCGGTGGCGTGTCACAGCTGATTAGCAATGGCCACAGGGT |
| | | GTTAGTTGAGACAGGAGCTGGCCTGGGCTCCGGCTTTGAAAATGAAGCGTATGAAAGTGCGG |
| | | GGGCGGAGATTATTGCCGATCCGAAACAAGTGTGGGACGCGGAGATGGTTATGAAGGTAAAG |
| | | GAGCCGCTGCCGGAGGAGTATGTCTACTTCCGTAAGGGTCTTGTTTTGTTCACTTACCTTCAT |
| | | CTGGCAGCGGAGCCGGAACTGGCCCAGGCACTTAAAGACAAAGGCGTGACCGCCATAGCAT |
| | | ACGAAACGGTGTCGGAAGGCCGTACTCTGCCGTTATTAACGCCCATGTCTGAGGTGGCAGGA |
| | | CGCATGGCGGCTCAAATTGGCGCGCAGTTCCTGGAAAAACCCAAGGGTGGCAAAGGCATTTT |
| | | ACTGGCCGGCGTGCCGGGCGTTTCGCGTGGCAAAGTGACCATTATAGGTGGGGGCGTGGTG |
| | | GGCACCAATGCGGCCAAGATGGCGGTTGGTCTGGGCGCAGACGTAACCATTATTGACCTTAA |
| | | TGCGGATCGGCTGAGACAGCTGGATGATATTTTTGGGCATCAGATTAAAACCCTGATTAGCAA |
| | | CCCGGTGAACATCGCCGACGCTGTGGCGGAGGCCGATCTTTTGATTTGCGCCGTCCTGATAC |
| | | CAGGAGCGAAAGCCCCGACCTTGGTGACGGAAGAAATGGTGAAACAGATGAAGCCGGGCTC |
| | | CGTAATTGTTGACGTGGCAATCGACCAAGGCGGAATTGTTGAAACCGTTGATCATATCACGAC |
| | | CCATGATCAACCCACGTACGAGAAACATGGCGTCGTCCATTACGCTGTCGCTAATATGCCGG |
| | | GCGCGGTGCCTCGCACTTCGACAATTGCCCTGACTAATGTCACCGTTCCCTACGCGCTGCAG |
| | | ATCGCTAACAAGGGCGCGGTGAAAGCCCTGGCTGACAACACGGCCTTAAGGGCGGGCTTAA |
| | | ATACGGCGAACGGCCATGTTACCTACGAAGCAGTGGCCCGCGACCTTGGATATGAATATGTC |
| | | CCGGCCGAAAAAGCCCTGCAGGATGAGAGTAGCGTGGCTGGAGCCTAAcgtctgacggcgataacgg |
| | | atgtttccatggacgctttaatctgggatggtttaataagctaagtgggatgaaag |
| | | gaggtaaggaATGGCGGTGGATTCTCCG |
| | | GATGAACGCCTTCAGCGCCGCATTGCGCAGCTATTTGCGGAAGACGAGCAGGTGAAAGCGG |
| | | CACGTCCGCTGGAGGCGGTGAGCGCAGCAGTCAGCGCACCCGGCATGAGGCTGGCACAAAT |
| | | CGCTGCGACCGTGATGGCAGGCTATGCCGACCGTCCGGCCGCAGGCCAGCGCGCATTTGAG |
| | | CTAAACACCGATGATGCCACGGGCCGGACGAGCTTACGCCTCCTGCCCCGTTTTGAAACTAT |
| | | TACCTATCGCGAACTGTGGCAGCGAGTGGGCGAAGTTGCGGCAGCGTGGCATCATGATCCC |
| | | GAAAATCCACTGCGTGCCGGCGATTTTGTGGCCCTGCTGGGTTTTACATCGATAGATTACGCA |
| | | ACACTGGATCTGGCGGACATTCATTTAGGGGCCGTGACAGTACCGCTGCAAGCGTCTGCGGC |
| | | GGTTAGCCAACTGATTGCAATTCTAACCGAAACCAGCCCTCGGCTCCTTGCGTCAACCCCGG |
| | | AACATCTGGATGCGGCGGTGGAATGTTTATTGGCCGGTACAACACCGGAACGCCTGGTGGTT |
| | | TTTGACTATCACCCCGAAGACGATGATCAGCGCGCGGCCTTTGAAAGTGCGCGCCGACGCTT |
| | | GGCCGACGCAGGCTCGCTGGTGATTGTTGAGACGCTCGATGCTGTGCGCGCGCGCGGACGT |
| | | GATCTACCGGCAGCACCCCTGTTTGTCCCGGACACAGACGACGATCCACTGGCCCTGCTGAT |
| | | TTATACGAGCGGGAGTACCGGCACCCCTAAAGGCGCGATGTACACCAATCGCCTGGCGGCAA |
| | | CCATGTGGCAAGGTAACAGCATGCTTCAGGGCAATAGCCAACGCGTTGGTATCAACCTTAATT |
| | | ATATGCCAATGTCACATATTGCAGGACGCATCTCGTTGTTTGGAGTACTTGCACGCGGTGGCA |
| | | CCGCATACTTTGCGGCGAAATCCGATATGTCCACGTTATTCGAGGATATTGGCTTGGTACGCC |
| | | CAACGGAGATTTTCTTTGTGCCACGCGTATGTGATATGGTGTTCCAGCGCTATCAAAGCGAAC |
| | | TGGATCGCCGCTCTGTAGCAGGTGCGGATTTAGATACACTGGACCGAGAAGTGAAGGCAGAC |
| | | CTGCGTCAGAACTACCTGGGTGGTCGCTTTCTCGTTGCGGTCGTGGGTAGCGCACCCTTGGC |
| | | AGCTGAAATGAAAACGTTCATGGAATCAGTACTGGATTTGCCATTACATGACGGCTATGGCTC |
| | | AACCGAAGCCGGCGCAAGCGTACTGCTTGATAACCAGATTCAGCGCCCTCCGGTTCTGGACT |
| | | ACAAGCTGGTGGATGTTCCAGAACTCGGTTATTTTCGTACTGATAGGCCGCACCCGCGCGGT |
| | | GAACTCTTATTAAAGGCAGAAACTACTATTCCGGGCTATTATAAACGTCCGGAAGTGACCGCG |
| | | GAAATCTTTGACGAGGATGGTTTTTACAAAACTGGCGATATCGTAGCGGAGTTAGAGCACGAC |
| | | CGCCTGGTATACGTGGACCGCCGAAATAACGTGCTCAAGCTGTCGCAGGGAGAGTTCGTGAC |
| | | TGTAGCCCACCTAGAAGCCGTGTTTGCATCCAGCCCGTTGATCCGACAGATTTTTATCTACGG |
| | | TAGTTCTGAGCGGAGCTACCTGTTGGCAGTAATTGTGCCGACCGATGACGCGCTGCGGGGAC |
| | | GCGATACCGCCACCCTGAAGTCCGCTTTGGCGGAATCGATTCAACGCATCGCGAAAGATGCA |
| | | AATCTGCAGCCGTATGAAATACCGCGCGATTTCCTGATCGAAACAGAACCGTTTACGATCGCA |
| | | AATGGTCTGCTTTCGGGCATTGCCAAACTTTTACGTCCTAACCTGAAAGAGCGCTACGGCGCC |
| | | CAACTGGAGCAGATGTACACCGATCTAGCCACCGGGCAGGCAGATGAACTCTTAGCATTACG |
| | | TCGCGAAGCTGCGGACCTCCCGGTGCTCGAAACTGTTAGTCGCGCAGCAAAGGCGATGCTG |
| | | GGCGTCGCGAGTGCAGATATGCGCCCCGATGCCCATTTTACTGACCTGGGTGGCGATTCCCT |
| | | GTCTGCGCTGTCCTTTTCCAATCTGCTACACGAAATTTTCGGTGTAGAAGTGCCTGTAGGTGT |
| | | AGTCGTCAGCCCGGCAAACGAACTTCGCGATCTGGCGAATTACATTGAGGCCGAAAGGAACA |
| | | GTGGCGCGAAACGCCCCACTTTTACTAGCGTACACGGCGGTGGCTCCGAAATTCGCGCGGC |
| | | AGACCTCACTCTGGACAAATTTATTGATGCGCGCACCCTGGCCGCGGCAGATTCGATTCCTCA |
| | | CGCGCCTGTTCCGGCCCAGACGGTGCTGCTGACCGGCGCAAACGGGTACTTAGGACGCTTT |
| | | CTTTGTCTTGAATGGCTGGAACGGCTCGATAAAACGGGCGGTACGCTGATATGTGTTGTGCGT |
| | | GGAAGCGACGCGGCGGCGGCGCGCAAGCGCCTAGATTCAGCGTTCGATAGCGGAGATCCC |
| | | GGCTTGCTGGAACATTATCAACAGCTGGCCGCCCGCACCCTGGAAGTTCTAGCGGGGGATAT |
| | | TGGTGATCCTAATCTGGGCCTCGATGATGCCACATGGCAGCGTCTGGCAGAAACAGTTGACC |
| | | TGATCGTGCATCCGGCAGCGTTGGTGAACCACGTGCTGCCGTATACGCAATTGTTCGGTCCG |
| | | AATGTCGTCGGCACCGCAGAAATCGTACGTTTAGCTATCACTGCCCGACGTAAGCCTGTGAC |
| | | GTACTTGTCGACTGTAGGTGTGGCGGATCAGGTGGACCCCGCCGAATATCAGGAAGATTCGG |
| | | ATGTACGCGAGATGAGCGCCGTGCGCGTAGTGCGCGAAAGCTATGCCAATGGGTACGGTAA |
| | | CTCGAAATGGGCGGGTGAAGTGCTGTTACGCGAAGCACATGATCTGTGCGGTTTGCCGGTCG |
| | | CAGTATTTCGGAGCGATATGATCTTAGCGCACTCGCGCTACGCGGGCCAGCTGAATGTGCAG |
| | | GACGTGTTTACCCGTCTGATACTCTCACTGGTGGCGACTGGCATCGCACCGTATAGTTTCTAT |
| | | CGGACAGATGCGGATGGTAATCGACAGCGTGCACATTATGATGGCCTTCCCGCGGATTTTAC |
| | | GGCCGCCGCGATAACGGCGCTCGGAATCCAGGCTACGGAAGGCTTTCGCACTTATGACGTCT |
| | | TGAACCCCTATGACGATGGTATTTCTCTGGATGAGTTTGTTGACTGGCTCGTAGAAAGCGGTC |
| | | ACCCGATCCAGCGCATTACAGATTACTCTGATTGGTTTCATCGTTTTGAAACCGCGATTCGTGC |
| | | GTTACCAGAAAAACAGCGCCAAGCGTCAGTCCTACCGCTCTTGGATGCGTACCGGAATCCGT |
| | | GTCCGGCCGTTCGCGGTGCAATCTTACCGGCGAAAGAATTTCAAGCTGCCGTTCAAACCGCT |
| | | AAAATTGGCCCGGAACAGGACATTCCGCACCTTAGTGCCCCTCTGATTGATAAATATGTGAGC |
| | | GATCTGGAACTGCTGCAACTGCTCTAACCTGTAGAAATAATTTTGTTTAACTTTAATAAGGAGA |
| | | TATACCATGGGCAAGATTTACGGAATTTATATGGACCGCCCGCTTTCACAGGAAGAAAATGAA |
| | | CGGTTCATGACTTTCATATCACCTGAAAAACGGGAGAAATGCCGGAGATTTTATCATAAAGAA |
| | | GATGCTCACCGCACCCTGCTGGGAGATGTGCTCGTTCGCTCAGTCATAAGCAGGCAGTATCA |
| | | GTTGGACAAATCCGATATCCGCTTTAGCACGCAGGAATACGGGAAGCCGTGCATCCCTGATC |
| | | TTCCCGACGCTCATTTCAACATTTCTCACTCCGGCCGCTGGGTCATTGGTGCGTTTGATTCAC |
| | | AGCCGATCGGCATAGATATCGAAAAAACGAAACCGATCAGCCTTGAGATCGCCAAGCGCTTCT |
| | | TTTCAAAAACAGAGTACAGCGACCTTTTAGCAAAAGACAAGGACGAGCAGACAGACTATTTTTA |
| | | TCATCTATGGTCAATGAAAGAAAGCTTTATCAAACAGGAAGGCAAAGGCTTATCGCTTCCGCTT |
| | | GATTCCTTTTCAGTGCGCCTGCATCAGGACGGACAAGTATCCATTGAGCTTCCGGACAGCCAT |
| | | TCCCCATGCTATATCAAAACGTATGAGGTCGATCCCGGCTACAAAATGGCTGTATGCGCCGCA |
| | | CACCCTGATTTCCCCGAGGATATCACAATGGTCTCGTACGAAGAGCTTTTATAAcaaataaaacga |
| | | aaggctcagtcgaaagactgggcctttcgttttatctgttgtttgtcggtgaacgctctctactagag |
| | | tcacactggctcaccttcgggtgggcctttctgcgtttatacctagggcgttcggctgcggcgagcgg |
| | | tatcagctcactcaaaggcggtaatacggttatccacagaatcaggggataacgcaggaaagaacatg |
| | | tgagcaaaaggccagcaaaaggccaggaaccgtaaaaaggccgcgttgctggcgtttttccataggct |
| | | ccgcccccctgacaagcatcacgaaatctgacgctcaaatcagtggtggcgaaacccgacaggactat |
| | | aaagataccaggcgtttccccctggcggctccctcgtgcgctctcctgttcctgcctttcggtttacc |
| | | ggtgtcattccgctgttatggccgcgtttgtctcattccacgcctgacactcagttccgggtaggcag |
| | | ttcgctccaagctggactgtatgcacgaaccccccgttcagtccgaccgctgcgccttatccggtaac |
| | | tatcgtcttgagtccaacccggaaagacatgcaaaagcaccactggcagcagccactggtaattgatt |
| | | tagaggagttagtcttgaagtcatgcgccggttaaggctaaactgaaaggacaagttttggtgactgc |
| | | gctcctccaagccagttacctcggttcaaagagttggtagctcagagaaccttcgaaaaaccgccctg |
| | | caaggcggttttttcgttttcagagcaagagattacgcgcagaccaaaacgatctcaagaagatcatc |
| | | ttattaatcagataaaatatttctagatttcagtgcaatttatctcttcaaatgtagcacctgaagtc |
| | | agccccatacgatataagttgttactagtgcttggattctcaccaataaaaaacgcccggcggcaacc |
| | | gagcgttctgaacaaatccagatggagttctgaggtcattactggatctatcaacaggagtccaagcg |
| | | agctctcgaaccccagagtcccgctcagaagaactcgtcaagaaggcgatagaaggcgatgcgctgcg |
| | | aatcgggagcggcgataccgtaaagcacgaggaagcggtcagcccattcgccgccaagctcttcagca |
| | | atatcacgggtagccaacgctatgtcctgatagcggtccgccacacccagccggccacagtcgatgaa |
| | | tccagaaaagcggccattttccaccatgatattcggcaagcaggcatcgccatgggtcacgacgagat |
| | | cctcgccgtcgggcatgcgcgccttgagcctggcgaacagttcggctggcgcgagcccctgatgctct |
| | | tcgtccagatcatcctgatcgacaagaccggcttccatccgagtacgtgctcgctcgatgcgatgttt |
| | | cgcttggtggtcgaatgggcaggtagccggatcaagcgtatgcagccgccgcattgcatcagccatga |
| | | tggatactttctcggcaggagcaaggtgagatgacaggagatcctgccccggcacttcgcccaatagc |
| | | agccagtcccttcccgcttcagtgacaacgtcgagcacagctgcgcaaggaacgcccgtcgtggccag |
| | | ccacgatagccgcgctgcctcgtcctgcagttcattcagggcaccggacaggtcggtcttgacaaaaa |
| | | gaaccgggcgcccctgcgctgacagccggaacacggcggcatcagagcagccgattgtctgttgtgcc |
| | | cagtcatagccgaatagcctctccacccaagcggccggagaacctgcgtgcaatccatcttgttcaat |
| | | catgcgaaacgatcctcatcctgtctcttgatcagatcatgatcccctgcgccatcagatccttggcg |
| | | gcaagaaagccatccagtttactttgcagggcttcccaaccttaccagagggcgccccagctggcaat |
| | | tccgacgtcgacaccatcgaatggtgcaaaacctttcgcggtatggcatgatagcgcccggaagagag |
| | | tcaattcagggtggtgaatgtgaaaccagtaacgttatacgatgtcgcagagtatgccggtgtctctt |
| | | atcagaccgtttcccgcgtggtgaaccaggccagccacgtttctgcgaaaacgcgggaaaaagtggaa |
| | | gcggcgatggcggagctgaattacattcccaaccgcgtggcacaacaactggcgggcaaacagtcgtt |
| | | gctgattggcgttgccacctccagtctggccctgcacgcgccgtcgcaaattgtcgcggcgattaaat |
| | | ctcgcgccgatcaactgggtgccagcgtggtggtgtcgatggtagaacgaagcggcgtcgaagcctgt |
| | | aaagcggcggtgcacaatcttctcgcgcaacgcgtcagtgggctgatcattaactatccgctggatga |
| | | ccaggatgccattgctgtggaagctgcctgcactaatgttccggcgttatttcttgatgtctctgacc |
| | | agacacccatcaacagtattattttctcccatgaagacggtacgcgactgggcgtggagcatctggtc |
| | | gcattgggtcaccagcaaatcgcgctgttagcgggcccattaagttctgtctcggcgcgtctgcgtct |
| | | ggctggctggcataaatatctcactcgcaatcaaattcagccgatagcggaacgggaaggcgactgga |
| | | gtgccatgtccggttttcaacaaaccatgcaaatgctgaatgagggcatcgttcccactgcgatgctg |
| | | gttgccaacgatcagatggcgctgggcgcaatgcgcgccattaccgagtccgggctgcgcgttggtgc |
| | | ggatatctcggtagtgggatacgacgataccgaagacagctcatgttatatcccgccgttaaccacca |
| | | tcaaacaggattttcgcctgctggggcaaaccagcgtggaccgcttgctgcaactctctcagggccag |
| | | gcggtgaagggcaatcagctgttgcccgtctcactggtgaaaagaaaaaccaccctggcgcccaatac |
| | | gcaaaccgcctctccccgcgcgttggccgattcattaatgcagctggcacgacaggtttcccgactgg |
| | | aaagcgggcagtgagcgcaacgcaattaatgtaagttagcgcgaattgatctggtttgacagcttatc |
| | | atcgactgcacggtgcaccaatgcttctggcgtcaggcagccatcggaagctgtggtatggctgtgca |
| | | ggtcgtaaatcactgcataattcgtgtcgctcaaggcgcactcccgttctggataatgttttttgcgc |
| | | cgacatcataacggttctggcaaatattctgaaatgagctgttgacaattaatcatccggctcgtata |
| | | atgtgtggaattgtgagcggataacaatttca |
| 27 | E. coli | MKREEICRLLADKVNKLKNKENSLSELLPDVRLLYGETPFARTPVMYEPGIIILFSGHKIGYINERVF |
| | transcriptional | RYDANEYLLLTVPLPFECETYATSEVPLAGLRLNVDILQLQELLMDIGEDEHFQPSMAASGINSATL |
| | regulator (yqhC) | SEEILCAAERLLDVMERPLDARILGKQIIREILYYVLTGPCGGALLALVSRQTHFSLISRVLKRIENKY |
| | amino acid | TENLSVEQLAAEANMSVSAFHHNFKSVTSTSPLQYLKNYRLHKARMMIIHDGMKASAAAMRVGYE |
| | sequence | SASQFSREFKRYFGVTPGEDAARMRAMQGN |
| 28 | Mycobacterium | MSPITREERLERRIQDLYANDPQFAAAKPATAITAAIERPGLPLPQIIETVMTGYADRPALAQRSVEF |
| | marinum CAR | VTDAGTGHTTLRLLPHFETISYGELWDRISALADVLSTEQTVKPGDRVCLLGFNSVDYATIDMTLAR |
| | (UniProt: B2HN69) | LGAVAVPLQTSAAITQLQPIVAETQPTMIAASVDALADATELALSGQTATRVLVFDHHRQVDAHRAA |
| | | VESARERLAGSAVVETLAEAIARGDVPRGASAGSAPGTDVSDDSLALLIYTSGSTGAPKGAMYPR |
| | | RNVATFWRKRTWFEGGYEPSITLNFMPMSHVMGRQILYGTLQNGGTAYFVAKSDLSTLFEDLALV |
| | | RPTELTFVPRVWDMVFDEFQSEVDRRLVDGADRVALEAQVKAEIRNDVLGGRYTSALTGSAPISD |
| | | EMKAWVEELLDMHLVEGYGSTEAGMILIDGAIRRPAVLDYKLVDVPDLGYFLTDRPHPRGELLVKT |
| | | DSLFPGYYQRAEVTADVFDADGFYRTGDIMAEVGPEQFVYLDRRNNVLKLSQGEFVTVSKLEAVF |
| | | GDSPLVRQIYIYGNSARAYLLAVIVPTQEALDAVPVEELKARLGDSLQEVAKAAGLQSYEIPRDFIIE |
| | | TTPWTLENGLLTGIRKLARPQLKKHYGELLEQIYTDLAHGQADELRSLRQSGADAPVLVTVCRAAA |
| | | ALLGGSASDVQPDAHFTDLGGDSLSALSFTNLLHEIFDIEVPVGVIVSPANDLQALADYVEAARKPG |
| | | SSRPTFASVHGASNGQVTEVHAGDLSLDKFIDAATLAEAPRLPAANTQVRTVLLTGATGFLGRYLA |
| | | LEWLERMDLVDGKLICLVRAKSDTEARARLDKTFDSGDPELLAHYRALAGDHLEVLAGDKGEADL |
| | | GLDRQTWQRLADTVDLIVDPAALVNHVLPYSQLFGPNALGTAELLRLALTSKIKPYSYTSTIGVADQ |
| | | IPPSAFTEDADIRVISATRAVDDSYANGYSNSKWAGEVLLREAHDLCGLPVAVFRCDMILADTTWA |
| | | GQLNVPDMFTRMILSLAATGIAPGSFYELAADGARQRAHYDGLPVEFIAEAISTLGAQSQDGFHTY |
| | | HVMNPYDDGIGLDEFVDWLNESGCPIQRIADYGDWLQRFETALRALPDRQRHSSLLPLLHNYRQP |
| | | ERPVRGSIAPTDRFRAAVQEAKIGPDKDIPHVGAPIIVKYVSDLRLLGLL |

***

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—Additional Materials & Methods

### Phosphate-Citrate Buffer (10× Stock):

174 g/L K₂HPO₄
40 g/L (NH₄)₂HPO₄
17 g/L Citric acid
Adjusted to pH 7 with concentrated KOH
Autoclaved

### Phosphate-Citrate Buffer:

1× Phosphate-citrate buffer from stock
1×M9 Trace elements
0.1 mM CaCl₂
2 mM MgSO₄
MilliQ H₂O to desired volume

### Phosphate-Citrate Buffer+Glycerol+Yeast Extract:

1× Phosphate-citrate buffer from stock
2% w/v Glycerol
5 g/L Yeast extract
1×M9 Trace elements
0.1 mM CaCl₂)
2 mM MgSO₄
MilliQ H2O to desired volume

### Example 2—Biotransformation of Benzoic Acid to Benzylamine

Initial investigations into the synthesis of benzylamine utilised the Escherichia coli MG1655 strain and the Escherichia coli MG1655 Reduced Aromatic Aldehyde Reduction (RARE) strain (Kunjapur et al., 2014). The RARE strain was engineered to facilitate the synthesis of aromatic aldehydes by inhibiting the reduction of aromatic aldehydes. It was hypothesised by the present inventors that the use of this strain in the methods disclosed herein would contribute to the maximisation of benzylamine yield by reducing the interception of the benzaldehyde intermediate by endogenous enzymes.

To facilitate the bioconversion, a two-plasmid system was used, such that the carboxylic acid reductase (CAR) and ω-transaminase (ω-TA) could be orthogonally expressed with their respective ancillary enzymes. This allows for the tuning/balancing of the pathway by varying the concentration of the respective inducer whilst also providing potential insights into the behaviour of the system. Nocardia iowensis CAR (NiCAR) and the Bacillus subtilis phosphopantetheinyl transferase (Sfp) were cloned into the anhydrous-tetracycline (aTC) inducible pBbE2c (Lee et al., 2011) plasmid whilst Chromobacterium violaceum and Vibrio fluvialis ω-TA were cloned into pBbA1k (Lee et al., 2011) alongside the Bacillus subtilis alanine dehydrogenase (BsAlaDH).

E. coli MG1655 and E. coli MG1655 RARE cells were transformed with pBbE2c NiCAR_Sfp and pBbA1k CvTA_BsAlaDH (P3B-CvTA). Transformants were first grown as 5 mL LB starter cultures supplemented with appropriate antibiotics. After overnight growth, the starter cultures were used to inoculate an additional 5 mL LB supplemented with antibiotics and 1% w/v glycerol. These production cultures were grown at 37° C. until an OD₆₀₀ₙₘ of ~0.6, at which point protein production was induced by the addition of 1 mM IPTG and 25 nM aTC. The induced cultures were grown for a further 24 h at 30° C. After protein production, the resultant cell pellet was harvested, washed and resuspended to a final OD₆₀₀ₙₘ of 5 in 5 mL phosphate-citrate buffer (see Example 1 for recipe) supplemented with 50 mM benzoic acid, 1% w/v glycerol and 100 mM alanine. Samples were taken at 24 h and analysed by reversed phase ultra-performance liquid chromatography (UPLC).

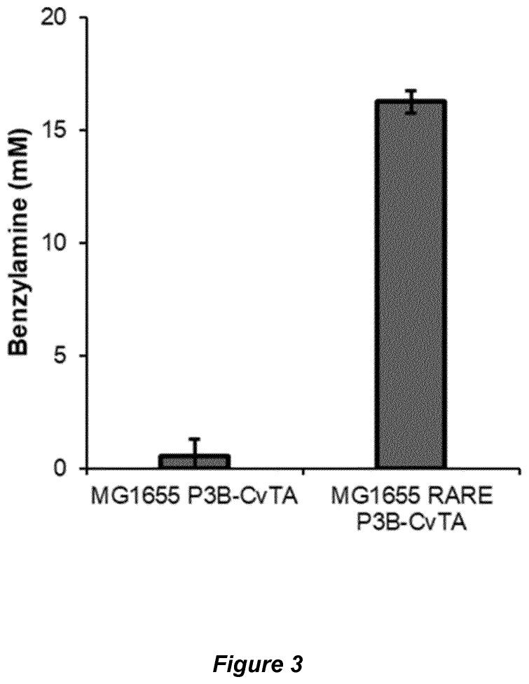

The titres observed after 24 h conclusively show that the benzylamine yields are significantly higher when using the MG1655 RARE strain (16.3±0.5 mM, 33% yield) compared to the base MG1655 strain (0.5±0.7 mM, 1% yield) (FIG. 3). Of the three replicates analysed, only one MG1655 P3B-CvTA clone produced any benzylamine (1.6 mM, 3.2% yield). While the biotransformation was investigated using biomass that was no longer actively growing, the results indicate that one or more of the endogenous aldo-keto reductases or alcohol dehydrogenases that have been knocked out in the MG1655 RARE strain are expressed and functional in the base MG1655 strain during the initial biomass accumulation phase of the process.

### Example 3—Biotransformation Optimisation

Small-scale biotransformation style reactions were performed to test the functionality of this biosynthetic route. A Design of Experiments (DoE) approach was used to identify key factors within the system that may be targeted for future experimental optimisation. In this initial design, inducer concentration (*−aTC & IPTG), ω-TA (Cv/Vf), cell loading, temperature and alanine concentration were explored as input variables (*=this variable was altered during the biomass production stage prior to the biotransformation reaction). The reactions were performed at a 5 mL scale whereby MG1655 RARE cells transformed with both plasmids were first grown and induced in shake-flask cultures. The resultant cell pellet was harvested and resuspended in 5 mL phosphate-citrate buffer (see Example 1 for recipe) containing 20 mM benzoic acid and alanine as specified by the experimental design. Samples were taken at 24 h & 48 h and analysed by reversed phase UPLC. In this experiment, the best performing set of conditions resulted in the production of 17.2 mM (1.8 g/L) benzylamine after 24 h (86% yield, 92% conversion).

Prior to the scaling up of the biotransformation reactions in a 5 L reactor, it was first decided to further expand on the initial scoping experiment to identify potential bottlenecks/barriers to the scale-up process. Building upon the scoping experiment, additional factors were included in the experimental design (pH & glycerol concentration) whilst the range of substrate (benzoic acid) concentration was significantly increased. Inducer concentrations, both of which were previously shown to have little significance, were removed from the design altogether whilst alanine concentration and temperature remained unchanged. A summary of the input variables can be seen in Table 1.

TABLE 1
Input variables investigated in the Design
of Experiments (DoE) guided biotransformation
of benzoic acid to benzylamine using MG1655 RARE whole
cell catalysts transformed with pBbE2c NiCAR_Sfp
and pBbA1k Cv/VfTA_BsAlaDH. The parentheses
refer to the variables where the concentration
was varied.

| Variable | Continuous or Categorical | Range | |
|---|---|---|---|
| pH | Continuous | 7-9 | |
| [Alanine] | Continuous | 10-100 | mM |
| ωTA | Categorical | CvTA/VfTA | |
| [Substrate] | Continuous | 50-150 | mM |
| Temperature | Continuous | 23-37° | C. |

| | | |
|---|---|---|
| [Glycerol] | Continuous | 1-2% |

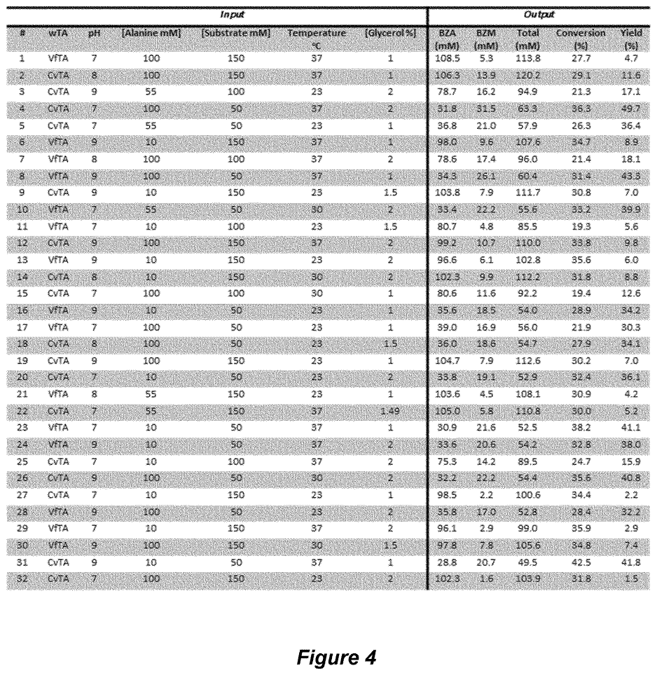

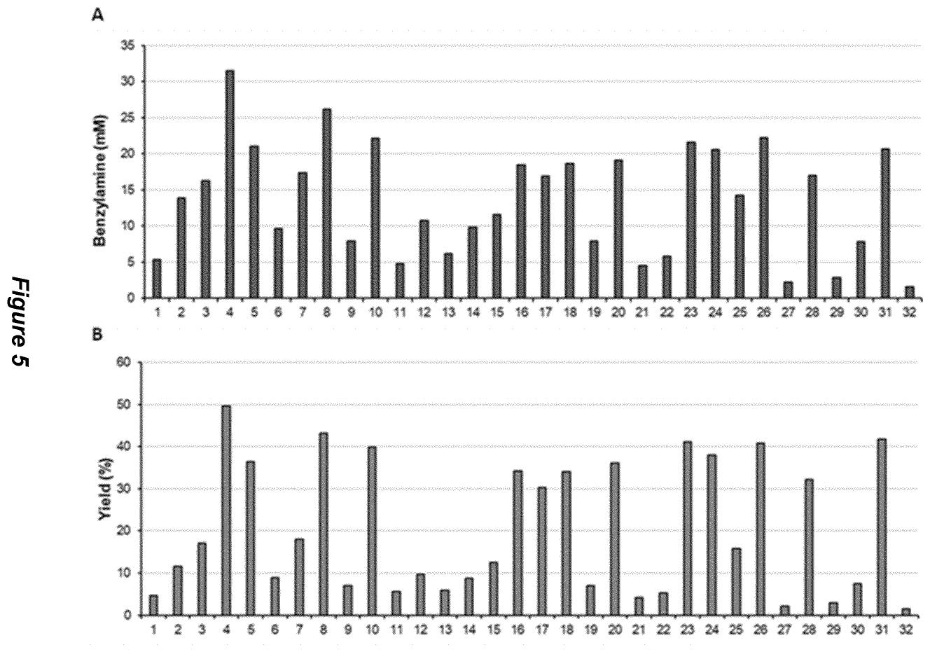

A 32-run experiment was designed in JMP Pro 16, using the custom design tool to optimally explore the experimental space, allowing for the characterisation of all main effects and second order interactions (full experimental design can be seen in FIG. 4). As described previously, MG1655 RARE transformed with pBbE2c NiCAR_Sfp and pBbA1k Cv/VfTA_BsAlaDH were grown in phosphate-citrate buffer (pH 7) supplemented with 2% glycerol and 5 g/L yeast extract at 37° C. in shake flasks and induced at an OD₆₀₀ₙₘ of 0.6 by the addition of 1 mM IPTG and 25 nM aTC (see Example 1 for media recipe). The induced cultures were grown for a further 16 h at 30° C. before cells were harvested by centrifugation. Cell pellets were washed three times in phosphate-citrate buffer, before finally being re-suspended at an OD₆₀₀ₙₘ of 5 in 5 mL in phosphate-citrate buffer supplemented according to the experimental design (FIG. 4). Biotransformation mixtures were then incubated with shaking at 23-37° C. for 24 h before samples were taken for UPLC analysis.

Of the reactions performed in the secondary biotransformation experiment, the highest benzylamine titre obtained was 31.5 mM (3.4 g/L, Run #4) (FIG. 5A). Interestingly, this reaction used the lowest amount of benzoic acid allowed within the experimental design. Conversely, the poorest performing reactions with regards to benzylamine titre (#27, 29 and 32) all used the highest starting benzoic acid concentration (150 mM). A potential explanation for this observation is that benzoic acid demonstrates toxicity to the cell, most likely by way of uncoupling ATP synthesis, as observed for many weak acids (Lou et al., 2007).

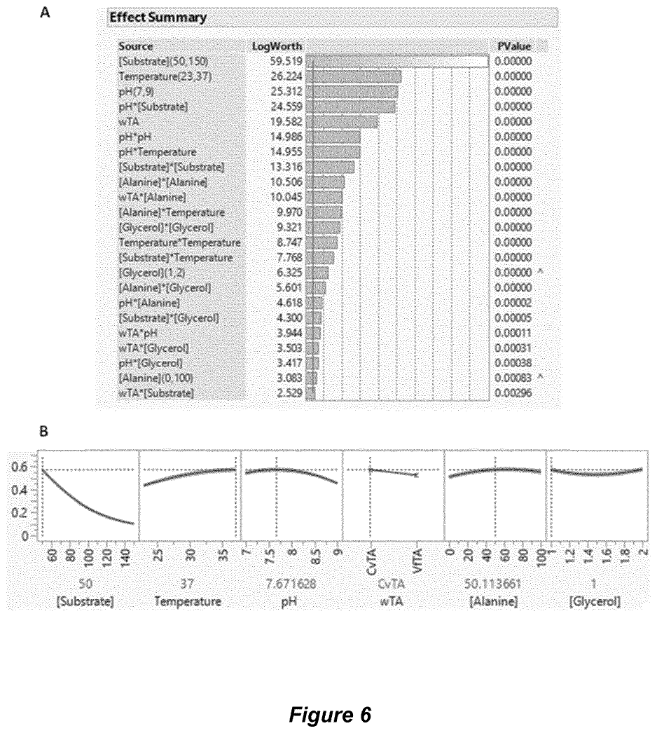

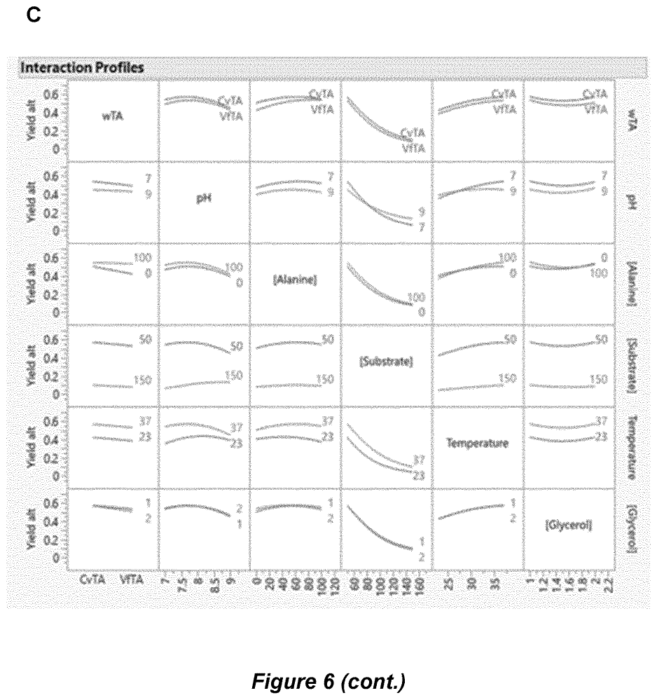

To gain further insights into how the variables affected benzylamine production, the % yields were calculated (FIGS. 4 & 5B) and used to fit a Generalised Linear Model (GLM) in JMP Pro 16. The resulting model was then simplified by the removal of non-significant terms. Of the remaining terms, by far the most significant was substrate concentration, followed by temperature and pH (FIG. 6A). With a functional, well-fitting model in place (χ²<0.0005) the model was used to predict the optimal combination of the input variables (FIG. 6B), in this instance the optimised configuration of conditions was predicted to facilitate a yield of 57%, a modest improvement over those seen in run #4. Both the prediction (FIG. 6B) and interaction (FIG. 6C) profilers indicate that using the lowest amount of substrate (50 mM) facilitates the best benzylamine yields, with significantly less benzylamine produced when using greater substrate concentrations.

### Example 4—Biotransformation Scale-Up

Prior to this point, all biotransformation reactions had been performed at 5 mL scale in sealed falcon tubes. To determine whether this process could be scaled, biotransformation reactions were performed in baffled shake-flasks at 10 mL, 100 mL and 1 L reaction volumes. Flasks were selected such that the reaction volume was 40% of the total volume of the vessel, in order to keep gas transfer rates consistent between replicates. Biomass (MG1655 RARE P3B-CvTA) was prepared as described in Example 3 and re-suspended to an OD₆₀₀ₙₘ of 5 in phosphate-citrate buffer (pH 7) supplemented with 50 mM benzoic acid and 1% w/v glycerol. All reactions were performed in duplicate. No alanine was added to the biotransformation buffer for this experiment. Samples were taken at 24, 72 and 120 h and diluted 1:20 in MilliQ H₂O before being analysed by reverse-phase UPLC.

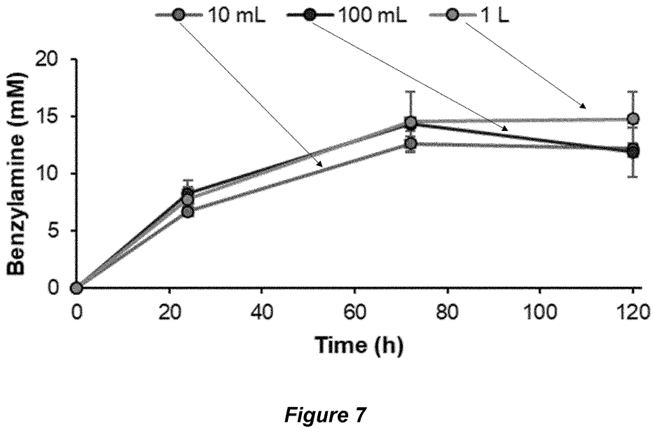

In this scale-up experiment, uniform benzylamine production was observed at 10 mL, 100 mL and 1 L reaction volumes (FIG. 7). This suggests that if key reaction parameters are maintained and scaled appropriately, the bioconversion of benzoic acid to benzylamine by MG1655 RARE P3B-CvTA should perform similarly as the process is developed further. Despite this, benzylamine titres were lower than those observed during previous similar experiments after 24 h (6.7-8.3 mM), reaching a high of 14.8±2.4 mM benzylamine (29.6% yield) after 120 h. This observation is most likely due to the absence of alanine supplementation, resulting in a reliance on the BsAlaDH mediated alanine recycling system to ensure sufficient amine donor availability for the amination of benzaldehyde. However, it is also possible that in the baffled shake-flask system the improved oxygenation results in the spontaneous (re) conversion of benzaldehyde to benzoic acid, thereby limiting substrate availability for the amination reaction. This phenomenon will be seen to a lesser extent when using screw-top falcon tubes which have poorer gas transfer and are likely to become pseudo-anaerobic over time.

### Example 5—Biomass Loadings Investigation

A DoE experiment was performed in which the bioconversion of 20 mM benzoic acid was investigated (see Example 3 for overview). In this scoping experiment, the biomass loading (OD₆₀₀ₙₘ 5-25) was shown not to significantly affect the benzylamine yields after 24 h (17.2 mM benzylamine, 86% yield, 92% conversion). Therefore, in subsequent experiments, loadings were fixed at an OD₆₀₀ₙₘ of 5, with a view that lower biomass loadings would be more cost efficient as the process scales.

When increasing the benzoic acid concentration in the biotransformation mixture to 50 mM (Example 3), benzylamine titres improved (15-25 mM) whilst both yields (~25-50%) and benzoic acid conversion were reduced. To further improve benzylamine titres, biomass loadings were once again revisited. Biomass (MG1655 RARE P3B-CvTA & MG1655 RARE P3B-VfTA) was prepared as described previously (Example 3). Biotransformation reactions were established as 5 mL cultures in 50 mL falcon tubes, with the biomass re-suspended to a final OD₆₀₀ₙₘ of 25, 50 or 100 in phosphate-citrate buffer (pH 7) supplemented with 50 mM benzoic acid and 1% glycerol. These reactions were also performed with and without the addition of 100 mM alanine to the biotransformation solution. Samples were taken at 24 h intervals and diluted 1:20 in MilliQ H₂O prior to analysis by reverse phase UPLC.

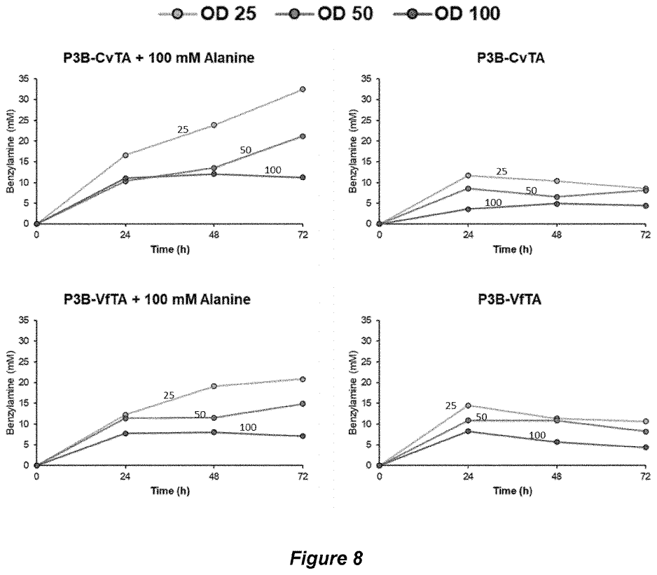

The resulting data (FIG. 8) suggests that significantly increasing the biomass loading at the start of the biotransformation reaction adversely affects benzylamine titres. Reactions that were established at a starting OD₆₀₀ₙₘ of 25 performed in-line with previous experiments in both the presence and absence of alanine, with a high of 32.5 mM benzylamine produced by MG1655 RARE P3B-CvTA supplemented with 100 mM alanine after 72 h. The same biocatalyst produced only 11.2 mM benzylamine with an initial loading of OD₆₀₀ₙₘ 100 in the presence of alanine. This observation was unexpected, as it was assumed that increased biomass would facilitate improved titres by virtue of greater catalyst availability, a reduced requirement per cell for co-factor regeneration and reduced relative concentration of benzoic acid per cell which could reduce the impact of potential ATP uncoupling.

### Example 6—Freeze-Dried Biomass Investigation

An investigation into the compatibility of this biocatalytic system with lyophilisation (freeze-drying) was performed. The ability to produce and subsequently lyophilise biomass at scale is advantageous as this eliminates the need for continuous culture of ‘fresh’ biomass, whilst also significantly reducing the costs associated with the storage and transport of said biomass.

Biomass (MG1655 RARE P3B-CvTA and P3B-VfTA) was again prepared as described previously (Example 3). Once the biomass had been harvested by centrifugation the pellet was washed three times using phosphate-citrate buffer (pH 7) supplemented with 5% glycerol. The pellet was then flash frozen in liquid nitrogen and attached to a freeze dryer. The biomass was dried for 3 days, until the pellet resembled a powder. At this point the dried cell weight was approximately a third of the wet cell weight measured prior to the freeze-drying process, indicating that the biomass was sufficiently dried. The dried biomass was then stored for a week at 4° C. prior to testing for catalytic activity.

The ability of the lyophilised biomass to convert benzoic acid to benzylamine was determined as described previously, at 5 mL scale in 50 mL falcon tubes. Dried biomass, 12.5 g/L (equivalent of OD₆₀₀ₙₘ 5), was added to phosphate-citrate buffer supplemented with 50 mM benzoic acid, 100 mM alanine and 1% glycerol. Samples were incubated at 37° C. with shaking with samples taken and diluted 1:20 in MilliQ H₂O prior to reverse phase UPLC analysis.

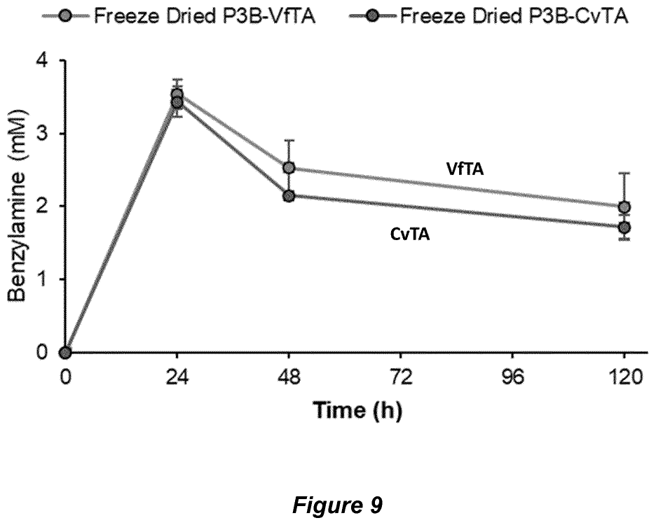

In both cases, the dried biomass retained low levels of catalytic activity (FIG. 9), with no significant differences observed between P3B-CvTA and P3B-VfTA pathways. However, the low benzylamine titres (P3B-CvTA 3.4±0.2 mM & P3B-VfTA 3.5±0.2 mM) indicate that the freeze-drying process has had a negative effect on the cell. Viability assays in which cell growth following the freeze-drying process is compared to that of cells prior to lyophilisation were not performed. Given that activity was still detected, further experiments will be performed in which different lyophilisation buffers/protectants are screened for their ability to preserve cell viability and catalytic activity.

### Example 7—Fed-Batch Fermentation Investigation

A potential strategy to further improve benzylamine titres is to exploit the ability of growing cells to convert benzoic acid to benzylamine in fed-batch fermentations. By supplementing cultures with benzoic acid during mid-late exponential growth, native ATP and NADPH co-factor regeneration pathways should still be functional, facilitating prolonged activity of the NiCAR/Sfp and ω-TA enzymes. This would also allow BsAlaDH to facilitate the regeneration of alanine (for use as an amine donor) using pyruvate generated by central metabolism. It was hoped that this would circumvent limitations associated with the biotransformation process used previously.

100 mL cultures were grown, in duplicate, in 500 mL baffled flasks. Starter cultures of E. coli MG1655 RARE freshly transformed with pBbE2c NiCAR_Sfp and pBbA1k VfTA_BsAlaDH were used to inoculate 100 mL of phosphate-citrate buffer (pH 7) supplemented with 2% w/v glycerol and 5 g/L yeast extract. The production cultures were grown at 37° C. in shake flasks and induced at an OD₆₀₀ₙₘ of 1 by the addition of 1 mM IPTG and 25 nM aTC, before the temperature was lowered to 30° C. After a further 2 h, an additional 1% w/v glycerol and 100 mM benzoic acid was added to each culture. Samples were taken at 16, 24 and 48 h post induction, the supernatant was diluted 1:20 in MilliQ water and the benzoic acid and benzylamine concentrations determined by UPLC. In this fed-batch experiment, after 48 h just 6.1±0.1 mM benzylamine was detected with 86.7±1.2 mM benzoic acid remaining unconverted in the supernatant (6.1% yield, 13.3% conversion).

Given the low yields, it was decided that further small-scale investigations would be performed prior to transitioning to 5 L fermentations. To better approximate a fed-batch fermentation set-up at small scale, a BioLector microbioreactor coupled with a RoboLector liquid handling platform was used to perform several miniaturised fed-batch fermentations in parallel. The use of these systems facilitated on-line monitoring of biomass, pH and dissolved oxygen (DO), each of which could be used to initiate triggers for automated dosing and/or sampling events such as induction, pH control, feeding and substrate addition.

As a first attempt at performing miniaturised fed-batch fermentation, the effect of the concentration and frequency of benzoic acid feeding was investigated. To mimic the biotransformation style reactions, 0.5 M benzoic acid was fed in a single ‘dose’ to final concentrations of 50, 100 and 150 mM during the late exponential phase of growth. Additional wells were also fed the equivalent of 100 and 150 mM benzoic acid in multiple 50 mM ‘doses’ at 4 h intervals in an effort to determine if the addition of large quantities of benzoic acid was detrimental to growth.

Starter cultures were prepared as described previously for fed-batch fermentations. The following day, the wells of an m2p labs FlowerPlate (MTP-48-BOH1) were filled with 800 μL phosphate-citrate buffer supplemented with 2% glycerol, 5 g/L yeast extract, 2 mM MgSO₄, 0.1 mM CaCl₂) and 1×M9 trace elements. Experimental wells were inoculated to an initial OD₆₀₀ₙₘ of 0.2 using the overnight starter cultures. The FlowerPlate was sealed with a breathable membrane and silicone cover, as per m2p protocols. The BioLector was set to maintain a temperature of 37° C., 80% humidity and a shaking speed of 1000 rpm.

Feed, pH and induction of each well were performed using the RoboLector, which monitored Biomass (G20), pH and DO with readings taken at 15 min intervals throughout the course of the experiment. The pH was maintained by the addition of 10 μL 1M KOH or 0.4 M H₂SO₄ when a measurement outside the permitted pH 6.8-7.2 range was detected. A feeding protocol was established whereby following the initial activation of the ‘Feed Active’ trigger (DO<30%), any time a DO>40% was observed, 10 UL of ‘Feed Solution’ (24% glycerol, 1.2% NH₄Cl, 50 mM MgSO₄) was added. Each well was permitted a maximum of 20 feeds and had a maximum permitted volume of 1500 μL. Induction of each culture was performed by the addition of 10 μL of 80× stocks of IPTG & aTC to achieve final concentrations of 1 mM and 25 nM respectively. Induction was triggered upon first reaching the G20 biomass value corresponding to an OD₆₀₀ₙₘ of 10. Benzoic acid was fed in varying amounts (50, 100 and 150 mM) once an OD₆₀₀ₙₘ of 18 had been reached, wells that were fed in increments were done so at 4 h intervals following the initial feed.

The experiment was run for a total of 48 h, at which point the wells were sampled and clarified lysate diluted 1:20 in MilliQ H₂O for UPLC analysis. Quantification of benzoic acid and benzylamine was determined by fitting a standard curve using the peak area of known concentrations of a standard. Again, benzylamine titres were low (<5 mM) for each of the conditions investigated, with a single benzoic acid feed at a final concentration of 100 mM yielding 4.7 mM benzylamine (FIG. 9). Interestingly, in each instance significant amounts of benzoic acid had been consumed. It is not clear whether this was lost to precipitation, metabolised by the cell, or converted into other by-products. No unidentified peaks were found by UPLC, with no significant differences observed in the concentration of known compounds seen between the control and experimental cultures.

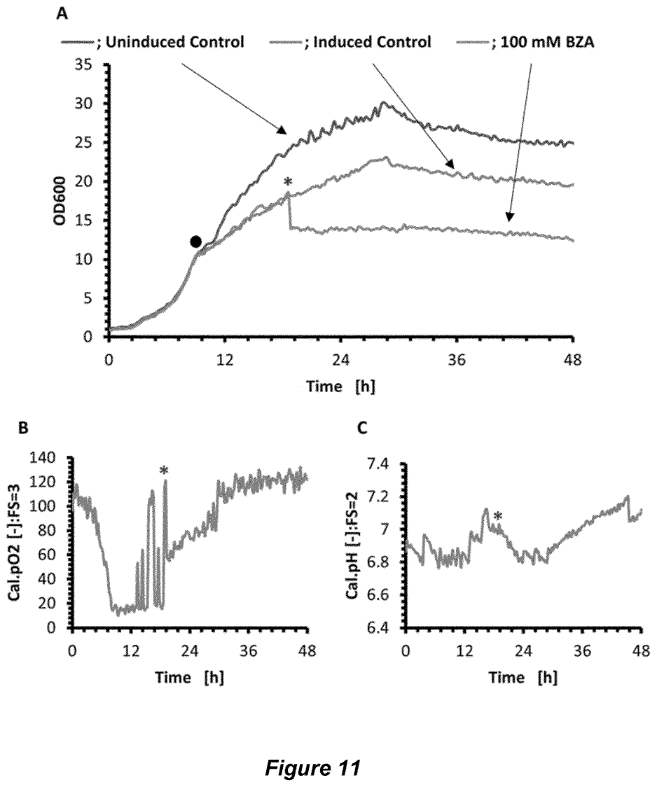

Comparison of biomass (OD₆₀₀ₙₘ) obtained for un-induced, induced and benzoic acid fed cultures demonstrate both the metabolic burden of induction and the addition of benzoic acid (FIG. 11A).

Upon addition of benzoic acid, a dilution effect was seen accounting for the observed drop in OD₆₀₀ₙₘ, however unlike in the induced control which continued to grow, the OD₆₀₀ₙₘ did not subsequently increase. This observation was confirmed when looking at the DO plot (FIG. 11B) which showed the DO beginning to rapidly rise after benzoic acid dosing, indicating that the cells were no longer respiring despite the continued addition of feed solution. This suggests that dosing at such high concentrations may result in some toxicity to the cell, potentially by way of uncoupling ATP synthesis (Lou et al., 2007). As such, co-factor synthesis and alanine recycling pathways will no longer be functional, potentially explaining the low titres that were observed in these fed-batch experiments.

### Example 8—Single Plasmid for the Benzylamine Production Cascade

A dual plasmid approach was used in previous examples (e.g., Example 2) where enzymes involved in the production of benzylamine were expressed by two separate plasmids (e.g., pBbE2c NiCAR_Sfp and pBbA1k CvTA_BsAlaDH).

A novel plasmid was designed and produced. The plasmid pBbA1-P3B-CvTA_BsAla_DH_NiCAR_Sfp (FIG. 12) was engineered to contain the following recombinant genes: w-transaminase (ω-TA) from Chromobacterium violaceum (CvTA); alanine dehydrogenase (AlaDH) from Bacillus subtilis (BsAlaDH); carboxylic reductase (CAR) from Nocardia iowensis (NiCAR) with its partner protein 4′-phosphopantetheinyl transferase (Sfp) from Bacillus subtilis.

### Plasmid Production and Purification

A glycerol stock of E. coli K-12 MG1655 RARE containing pBbA1k CvTA_BsAlaDH_NiCAR_Sfp was defrosted and used to streak an LBA plate supplemented with 50 μg/ml kanamycin. A single colony was used to inoculate 12 mL of LB medium containing 50 μg/ml kanamycin. The culture was incubated at 37° C. overnight with 180 rpm agitation. Plasmid recovery and purification was performed using the QIAprep Spin miniprep.

Following purification, plasmid concentration was determined, and the sample was diluted to 30 ng/μL plasmid DNA. This was sent for whole plasmid (nanopore) sequencing at Source Bioscience.

Nanopore 30 sequencing was successfully completed with 7203 partial reads of the plasmid DNA. The reconstructed sequence generated by the nanopore software was aligned with the expected sequence in Snapgene.

The alignment showed that the gene sequences for NICAR, CvTA and BsAlaDH were 100% correct. However, the gene sequence for the sfp gene showed mutations which were previously identified by Sanger sequencing. The mutations detected were as follows: A67T; C249T; T255C; C264T; C276T; G292T; T294C; T373G and C639T. Most of them are silent mutations, however three led to amino acid substitutions (T23S; G98C; S125A), but the sfp gene was demonstrated to be functional. An additional mutation was identified within the origin of replication (ori) of the plasmid, but this was not expected to affect the replication of the plasmid significantly. Therefore, no efforts were made to reverse the mutations.

### Example 9—Benzylamine Production Whole Cell Biocatalytic Cascade

### Experimental

### Culture Growth

Inoculate 10-15 mL LB medium containing the appropriate 1× antibiotic(s) with a single colony from a LB agar plate of an E. coli strain containing the required plasmid(s).

### Cultures

1 plasmid (pBbA1k-CvTA_BsAlaDH_NiCAR_sfp) in E. coli RARE (1R_2)—Kan^{R}
1 plasmid (pBbA1k_CvTA_BsAlaDH_NiCAR_sfp) in E. coli BL21 (1B_2)—Kan^{R}
2 plasmids (pBbE2c NiCAR_Sfp and pBbA1k CvTA_BsAlaDH) in E. coli RARE (2R_2)—Cam^{R} and Kan^{R}.
2 plasmids (pBbE2c NiCAR_Sfp and pBbA1k CvTA_BsAlaDH) in E. coli BL21 (2B_2)—Cam^{R} and Kan^{R}.

Starter cultures (10% vol) were used to inoculate 100 mL expression buffer medium containing the appropriate antibiotic. Cultures were incubated overnight at 37° C. at 180 rpm agitation. Once an OD 600 nm of ~0.6 was reached (1 h), the cultures were induced with 1 mM IPTG (pBbA1k) and 25 nM anhydrotetracycline (aTC; plasmid pBbE2c). Cultures were further incubated at 30° C. for 24 hours with 180 rpm agitation.

Cells were recovered by centrifugation at 8000 g for 25 min at 4° C. The cell pellets were washed three times in 1× sterile phosphate-citrate buffer, before being resuspended in 5 mL in phosphate-citrate buffer to an OD_{600 nm} of 5.

### Cascade Reactions

Three sets of experiments were set up (A, B and C), with each set containing four replicates:

Buffer A: 100 mM phosphate citrate buffer pH 7.0 containing 50 mM sodium benzoate and 100 mM alanine. This enabled a full cascade from benzoic acid to benzylamine with potential AlaDH activity. Reactions were set up with sodium benzoate, which had a higher water solubility than benzoic acid. This enabled a comparison between cascade reactions using whole cells from E. coli BL21 and E. coli RARE cultures expressing a one or two plasmid system.

Buffer B: 100 mM phosphate citrate buffer pH 7.0 containing 50 mM benzaldehyde and 100 mM alanine. This was designed to study only the second reaction from benzaldehyde to benzylamine (FIG. 13A). There is a possibility of seeing AlaDH activity also. This enabled a study of endogenous aldehyde dehydrogenase activity in each E. coli strain.

Buffer C: 100 mM phosphate citrate buffer pH 7.0 containing 50 mM benzaldehyde. No cascading reaction possible, so could only potentially see endogenous aldehyde dehydrogenase activity in each E. coli strain. This enabled an investigation of whether significant loss of benzaldehyde was present due to the action of endogenous enzyme(s) within the E. coli strains.

Full Cascade Reaction from Benzoic Acid to Benzylamine—Buffer A
1. 1 plasmid in E. coli BL21, 50 mM benzoic acid, 100 mM alanine, 2% glycerol
2. 1 plasmid in E. coli RARE, 50 mM benzoic acid, 100 mM alanine, 2% glycerol
3. 2 plasmids in E. coli BL21, 50 mM benzoic acid, 100 mM alanine, 2% glycerol
4. 2 plasmids in E. coli RARE, 50 mM benzoic acid, 100 mM alanine, 2% glycerol
   Benzaldehyde to Benzylamine—TA Activity with Alanine—Buffer B
5. 1 plasmid in E. coli BL21, 50 mM benzaldehyde, 100 mM alanine, 2% glycerol
6. 1 plasmid in E. coli RARE, 50 mM benzaldehyde, 100 mM alanine, 2% glycerol
7. 2 plasmids in E. coli BL21, 50 mM benzaldehyde, 100 mM alanine, 2% glycerol
8. 2 plasmids in E. coli RARE, 50 mM benzaldehyde, 100 mM alanine, 2% glycerol, 5% DMSO

### Reverse Reaction Benzaldehyde to Benzoic Acid—Low to No TA Activity—Buffer C

9. 1 plasmid in E. coli BL21, 50 mM benzaldehyde, 2% glycerol
10. 1 plasmid in E. coli RARE, 50 mM benzaldehyde, 2% glycerol
11. 2 plasmids in E. coli BL21, 50 mM benzaldehyde, 2% glycerol
12. 2 plasmids in E. coli RARE, 50 mM benzaldehyde, 2% glycerol, 5% DMSO

Reactions were incubated at 37° C. for 24 hours. Glycerol (2%) was added to each reactions as the cells were in a semi-resting state and potentially could use it as a carbon source. For reactions that did not contain exogenous alanine, there should be some benzylamine formation since the cells produce small amounts of alanine and other potential amine donors.

### Analytical Methods

After the cascade reactions, samples were diluted 20-fold in Milli Q water, followed by centrifugation for 10 min at 13,000 rpm. The supernatants (2 mL) were analysed for benzoic acid, benzylamine and benzaldehyde content by HPLC using a Luna® 3 μm C18(2) 100 Å, LC Column 100.

### Results and Discussion

### Full Cascade

Whole cell reactions for the full cascade reactions showed the highest benzylamine titres when the E. coli RARE strain was used (FIG. 13B). This generated 2.4 g/L benzylamine, equivalent to a 46% conversion. The E. coli RARE plasmid is a superior host as it has undergone the genomic knockout of some of its endogenous aldehyde dehydrogenases to minimise the chance of degradation of the benzaldehyde intermediate. The data also showed that whole cells containing only a single plasmid system generated higher benzylamine titres than those with the two-plasmid system (FIG. 13B). This could be in part due to the requirement of two antibiotics for the two-plasmid system, which likely reduces the fitness of the strain.

### Benzaldehyde to Benzylamine Reaction

Reactions with the second step only (benzaldehyde to benzylamine in Buffer B) showed there was very little endogenous aldehyde dehydrogenase activity seen where benzaldehyde was oxidised back to benzoic acid (FIG. 13C). There was not much difference in the benzoic acid production titres between the four reaction conditions, suggesting the impact of AlaDH activity on benzylamine production is relatively low.

Overall, the titres of benzylamine production were quite similar between the four reaction conditions. Slightly higher titres were seen in the one plasmid system in the E. coli RARE strain.

### Reverse Reaction of Benzaldehyde to Benzoic Acid

In the absence of exogenous alanine, there was very little evidence of the back reaction of benzaldehyde to benzoic acid with either strain (FIG. 13D). There was also very little evidence of the transamination of benzaldehyde to benzylamine as no exogenous alanine (required amine donor) was present. This is positive as it shows little interference from endogenous E. coli enzymes. However, it is possible that the expressed CAR enzyme will reduce benzoic acid back to benzaldehyde, so the true level of aldehyde dehydrogenase activity is not known.

### Conclusions

Comparative studies were performed with two E. coli strains and a one vs two plasmid system to produce all 4 recombinant enzymes necessary to generate benzylamine from benzoic acid. Control reactions were also performed to determine the level of interference of endogenous E. coli alanine dehydrogenase enzymes that could impact on benzylamine titres.

Overall, the best conditions for working with whole cells as biocatalysts was to use the E. coli RARE strain expressing the one plasmid system. This requires only one antibiotic and showed around a 50% conversion within 24 h with no significant levels of by-products.

### Example 10—Effect of Alanine and Glucose on Benzylamine Titres

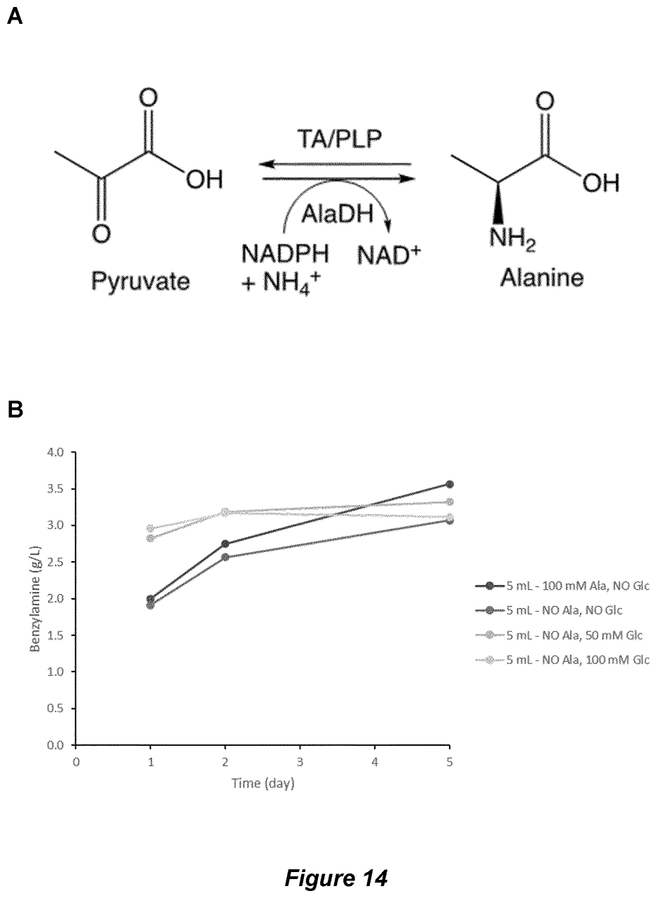

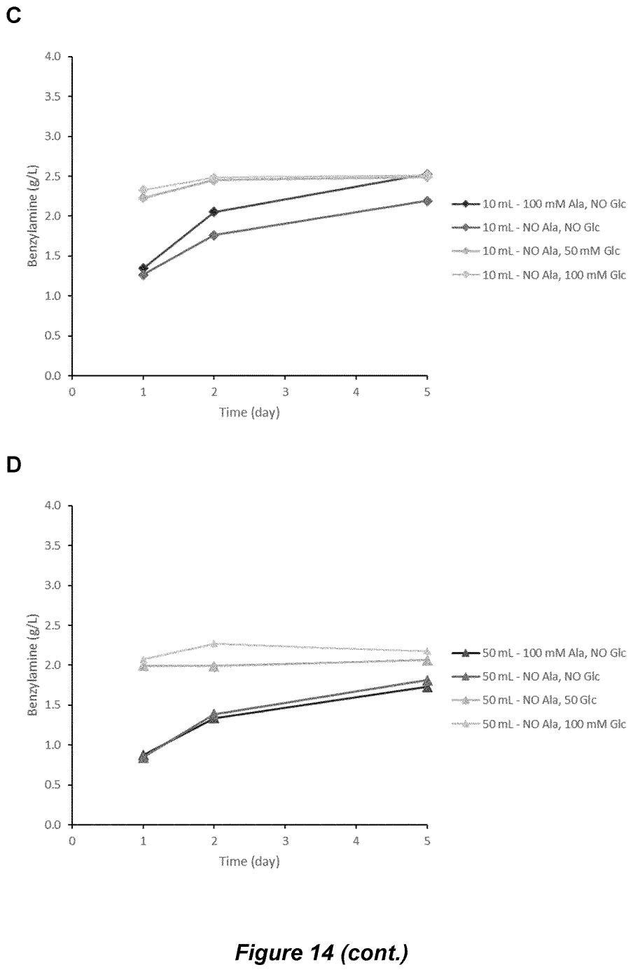

The benzylamine biocatalytic cascade reaction contains excess alanine (100 mM) as the amine donor via the action of an alanine dehydrogenase from Bacillus subtilis (BsAlaDH). This enzyme can also perform the reverse reaction of L-Ala synthesis from pyruvate and ammonium (reductive amination), with pyruvate coming from central E. coli metabolism via glucose utilisation (FIG. 14A). Therefore, E. coli could potentially not only synthesise benzylamine from benzoic acid, but it could supply one of the substrates alanine (or another alanine donor), in addition to regenerating the required ATP and NADPH. In this experiment, we investigate whether alanine could be solely supplied by the whole cell biocatalyst. To support alanine production, glucose will be supplied to the medium to increase the production of pyruvate.

### Experimental

### Stock Solutions and Culture Media

Alanine stock: 500 mM in phosphate-citrate buffer.

Sodium benzoate stock: 500 mM in phosphate-citrate buffer.

Phosphate-citrate buffer (10× stock): 174 g/L K₂HPO₄; 40 g/L (NH₄)₂HPO₄ and 17 g/L citric acid pH 7.0.

Buffer was diluted to 1× and autoclaved.

LB: 10 g/L tryptone; 5 g/L yeast extract and 10 g/L NaCl.

LB agar: 10 g/L tryptone; 5 g/L yeast extract, 10 g/L NaCl and 15 g/L agar.

Expression buffer: 1× Phosphate-citrate buffer, 2% w/v glycerol, 5 g/L Yeast extract, 1×M9 Trace elements (Chapter 4), 0.1 mM CaCl₂·2H₂O and 2 mM MgSO₄·7H₂O. Adjusted to pH 7.0 with KOH.

### Culture Growth

Inoculate 10-15 mL LB medium containing 50 μg/ml kanamycin with a single colony from a LB agar plate of an E. coli strain MG1655 RARE containing pBbA1k CvTA_BsAlaDH_NiCAR_Sfp. Starter cultures (10% vol) were used to inoculate 110 mL expression buffer medium containing 50 μg/ml kanamycin in 0.5 L flasks. Cultures were incubated overnight at 37° C. at 180 rpm agitation. Once an OD 600 nm of ~0.6 was reached, the cultures were induced with 1 mM IPTG. Cultures were further incubated at 30° C. for 20 hours with 180 rpm agitation.

Cells were recovered by centrifugation at 8500 g for 25 min at 4° C. The cell pellets were washed three times in 1× sterile phosphate-citrate buffer, before being resuspended in phosphate-citrate buffer to an OD_{600 nm} of 5.

### Cascade Reactions

All reactions were set up in 100 mM phosphate citrate buffer pH 7.0 containing variable alanine and glucose concentrations, 50 mM sodium benzoate and whole cell biomass at OD_{600 nm} as defined in the reactions code below. Reactions were incubated at 37° C. for up to 5 days with 180 rpm agitation. The reaction volumes are shown in the reactions code section below. Samples were taken at 0.5, 1, 2, 3, 4, 5, 6, 21 and 24 h, with a few samples analysed after 8 days. Samples were analysed by HPLC.

### Reactions Code

1. Volume=5 mL; 100 mM Ala, no glucose
2. Volume=5 mL; No Ala, no glucose
3. Volume=5 mL; No Ala, 50 mM glucose
4. Volume=5 mL; No Ala, 100 mM glucose
5. Volume=10 mL; 100 mM Ala, no glucose
6. Volume=10 mL; No Ala, no glucose
7. Volume=10 mL; No Ala, 50 mM glucose
8. Volume=10 mL; No Ala, 100 mM glucose
9. Volume=50 mL; 100 mM Ala, no glucose
10. Volume=50 mL; No Ala, no glucose
11. Volume=50 mL; No Ala, 50 mM glucose
12. Volume=50 mL; No Ala, 100 mM glucose

### Analytical Methods

After the cascade reactions, samples were diluted 20-fold in Milli Q water, followed by centrifugation for 10 min at 13,000 rpm. The supernatants (2 mL) were analysed for benzoic acid, benzylamine and benzaldehyde content by HPLC using a Luna® 3 μm C18(2) 100 Å, LC Column 100.

### Results and Discussion

In these experiments, the absence of exogenously supplied alanine and glucose gives slightly lower or around the same benzylamine titres as when alanine is present (FIG. 14B). This suggests the whole cell biocatalyst is capable of generating sufficient alanine, or another amine donor, for benzylamine synthesis. Interestingly, the presence of glucose, but not alanine, leads to about the same benzylamine titres as the other experiments (3.3 g/L in 5 mL), however this titre was achieved more rapidly.

These same trends were apparent in the 10 mL (FIG. 14C) and 50 mL (FIG. 14D) reactions. As in previous experiments, the total benzylamine production decreased as the reaction volume increased.

The increased reaction rate of benzylamine production in the absence of alanine suggests at least one of the two catalytic enzymes may have been inhibited by high levels of alanine in the reaction. The reaction acceleration in the presence of glucose may be due to increased metabolic activity of the whole cell biocatalyst. This in turn may increase the production of the amine donor, NADPH and/or ATP.

### Conclusions

The overall benzylamine highest titres obtained in this experiment are 3.5 g/L after 5 days. Results suggest benzylamine production reactions can work without exogenous alanine addition. The presence of glucose further elevates the overall reaction rate.

### Example 11—Effect of E. coli Strain on Benzylamine Production

### Introduction

Prior experiments focused on benzylamine production using an E. coli strain K-12 MG1655 RARE. The main characteristic of this strain is that genes for aldehyde reduction were deleted (RARE—reduced aromatic aldehyde reduction), which increases benzylamine production compared to control strain BL21 (DE3). Another similar strain is available that has also been genetically engineered to decrease the number of aromatic aldehyde reduction activity in the cell.

In this experiment, we aim to perform comparative benzylamine production using the one plasmid system with the three strains, to see if the new BL21(DE3) BZE strain could function as another host for scaled benzylamine production.

### Experimental

E. coli BL21(DE3) BZE Description

The E. coli BL21(DE3) BZE strain was obtained by deleting several genes of the genome of E. coli BL21 (DE3). The following genes were knocked out: yqhC, yqhD, yahK, yeaE, dkgA and yjgB. These genes are related to the expression of endogenous aldo-keto reductases (AKRs) and alcohol dehydrogenases (ADHs), which react with the intermediate of the biocatalytic cascade producing an undesired by-product. Therefore, the deletion of these genes is expected to increase the production of benzylamine from benzoic acid.

### Transformations

The single plasmid pBbA1K-CvTA_BsAlaDH_NiCAR_Sfp was transformed into the following strains:
1. E. coli BL21(DE3) (control strain—no gene knockouts)
2. E. coli BL21(DE3) BZE

The transformation protocol is described in Chapter 1. Note: E. coli K-12 MG1655 RARE strain was previously transformed with the single plasmid pBbA1K-CvTA_BsAlaDH_NiCAR_Sfp. Cultures were plated onto LB agar containing 50 μg/ml kanamycin and incubated overnight at 37° C. Colonies were cultivated and checked for the presence of the plasmid, as described in Example 8.

### Stock Solutions and Culture Media

Sodium benzoate stock: 500 mM in phosphate-citrate buffer.

Phosphate-citrate buffer (10× stock): 174 g/L K₂HPO₄; 40 g/L (NH₄)₂HPO₄ and 17 g/L citric acid pH 7.0.

Buffer was diluted to 1× and autoclaved.

LB: 10 g/L tryptone; 5 g/L yeast extract and 10 g/L NaCl.

LB agar: 10 g/L tryptone; 5 g/L yeast extract, 10 g/L NaCl and 15 g/L agar.

Expression buffer: 1× Phosphate-citrate buffer, 2% w/v glycerol, 5 g/L Yeast extract, 1×M9 Trace elements (Chapter 4), 0.1 mM CaCl₂·2H₂O and 2 mM MgSO₄·7H₂O. Adjusted to pH 7.0 with KOH.

### Culture Growth

Inoculate 10-15 mL LB medium containing 50 μg/ml kanamycin with a single colony from a LB agar plate of each of the three E. coli strains: a) BL21(DE3), b) MG1655 RARE and c) BL21(DE3) BZE containing plasmid pBbA1k_CvTA_BsAlaDH_NiCAR_Sfp. Starter cultures (10% vol) were used to inoculate 110 mL expression buffer medium containing 50 μg/ml kanamycin in 0.5 L flasks. Cultures were incubated overnight at 37° C. at 180 rpm agitation. Once an OD_{600 nm} of ~0.6 was reached, the cultures were induced with 1 mM IPTG. Cultures were further incubated at 30° C. for 20 hours with 180 rpm agitation.

Cells were recovered by centrifugation at 8500 g for 25 min at 4° C. The cell pellets were washed three times in 1× sterile phosphate-citrate buffer, before being resuspended in phosphate-citrate buffer to an OD_{600 nm} of 5.

### Cascade Reactions

All reactions were set up in 100 mM phosphate citrate buffer pH 7.0 containing 100 mM glucose, 50 mM sodium benzoate (no alanine) and whole cell biomass at OD 600 nm of 5. Reactions (5 and 10 mL) were incubated at 37° C. for 24-72 h with 180 rpm agitation. Samples were analysed by HPLC for benzylamine, benzoic acid, benzylamine and benzyl alcohol (byproduct) content.

### Reactions Code:

1. E. coli BL21(DE3) (control); 5 mL reaction
2. E. coli K-12 MG1655 RARE; 5 mL reaction
3. E. coli BL21(DE3) BZE; 5 mL reaction
4. E. coli BL21(DE3) (control); 10 mL reaction
5. E. coli K-12 MG1655 RARE; 10 mL reaction
6. E. coli BL21(DE3) BZE; 10 mL reaction

### Analytical Methods

After the cascade reactions, samples were diluted 20-fold in Milli Q water, followed by centrifugation for 10 min at 13,000 rpm. The supernatants (4 mL) were analysed for benzoic acid, benzylamine and benzaldehyde content by UPLC using a Thermo Scientific™ Hypersil GOLD™ C18 Selectivity (3 mm×4 mm×100 mm).

### Results and Discussion

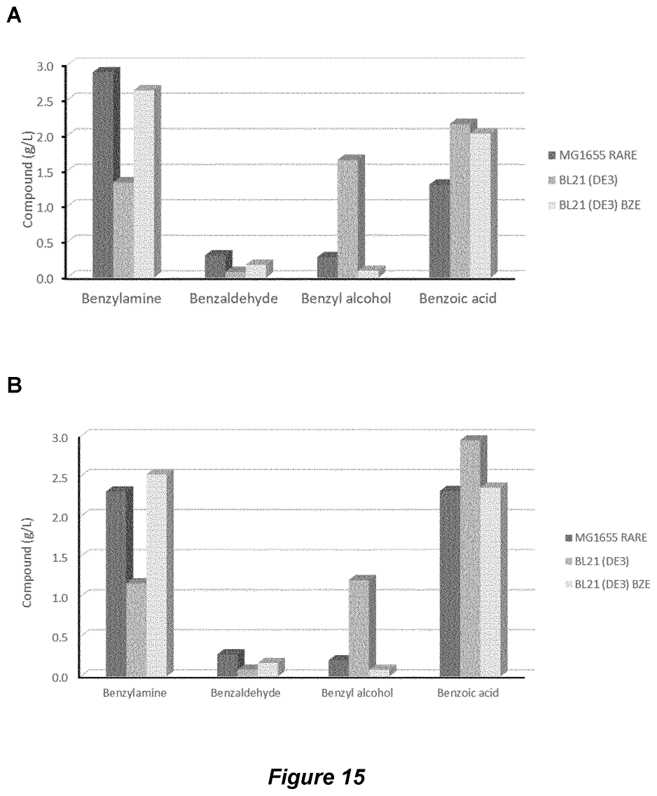

Biocatalytic reactions were incubated up to 3 days, but the titres after 24 h were similar. Therefore, the data below shows the products and remaining substrate after a 24 h reaction. The maximum titres obtained for the BL21(DE3) BZE strain were 2.63 (FIG. 15A) and 2.52 g/L benzylamine (FIG. 15B) for 5 mL and 10 mL reactions, respectively. This equates to over 50% conversion, with minimal by-product formation. Similar results were seen with the MG16555 RARE strain, while the control BL21(DE3) strain showed only around half the benzylamine titres. These results also mirrored the 10 mL reactions, with the same trends occurring.

The accumulation of small amounts of benzaldehyde in the reaction suggest that the benzaldehyde to benzylamine reaction is rate limiting. The presence of significant quantities of benzyl alcohol in the control BL21(DE3) strain compared to the knock-out strains shows the positive effects of the absence of many aldehyde reduction genes on minimising by-product formation. Reactions with the BL21(DE3) BZE strain showed the lowest benzyl alcohol production, and consequently the highest benzylamine production titres (10 mL reaction).

### Conclusions

E. coli strain BL21(DE3) BZE is a suitable alternative to the MG1655 RARE strain as a whole cell biocatalyst for amine production.

### REFERENCES

A number of publications are cited above in order to more fully describe and disclose the invention and the state of the art to which the invention pertains. Full citations for these references are provided below. The entirety of each of these references is incorporated herein.
1. Finnigan, W., Thomas, A., Cromar, H., Gough, B., Snajdrova, R., Adams, J. P., Littlechild, J. A., & Harmer, N. J. (2017). Characterization of carboxylic acid reductases as enzymes in the toolbox for synthetic chemistry. ChemCatChem, 9(6), 1005-1017. https://doi.org/10.1002/CCTC.201601249.
2. Fuchs, M., Tauber, K., Sattler, J., Lechner, H., Pfeffer, J., Kroutil, W., & Faber, K. (2012). Amination of benzylic and cinnamic alcohols via a biocatalytic, aerobic, oxidation-transamination cascade. RSC Advances, 2(15), 6262. https://doi.org/10.1039/c2ra20800h.
3. Kunjapur, A. M., Tarasova, Y., & Prather, K. L. J. (2014). Synthesis and accumulation of aromatic aldehydes in an engineered strain of Escherichia coli. Journal of the American Chemical Society, 136(33), 11644-11654. https://doi.org/10.1021/ja506664a.
4. Lee, T. S., Krupa, R. A., Zhang, F., Hajimorad, M., Holtz, W. J., Prasad, N., Lee, S. K., & Keasling, J. D. (2011). BglBrick vectors and datasheets: A synthetic biology platform for gene expression. Journal of Biological Engineering, 5(1), 12. https://doi.org/10.1186/1754-1611-5-12.
5. Li, S., Jendresen, C. B., Landberg, J., Pedersen, L. E., Sonnenschein, N., Jensen, S. I., & Nielsen, A. T. (2020). Genome-Wide CRISPRi-based identification of targets for decoupling growth from production. ACS Synthetic Biology, 9(5), 1030-1040. https://doi.org/10.1021/acssynbio.9b00143.
6. Liu, P., Xu, H., & Zhang, X. (2022). Metabolic engineering of microorganisms for L-alanine production. Journal of Industrial Microbiology and Biotechnology, 49(2), kuab057. https://doi.org/10.1093/jimb/kuab057.
7. Lou, P. H., Hansen, B. S., Olsen, P. H., Tullin, S., Murphy, M. P., & Brand, M. D. (2007). Mitochondrial uncouplers with an extraordinary dynamic range. The Biochemical Journal, 407(Pt 1), 129. https://doi.org/10.1042/BJ20070606.
8. Luo, Z. W., & Lee, S. Y. (2020). Metabolic engineering of Escherichia coli for the production of benzoic acid from glucose. Metabolic Engineering, 62, 298-311. https://doi.org/10.1016/J.YMBEN.2020.10.002.
9. Pandey, R. P., Casini, A., Voigt, C. A., & Gordon, D. B. (2021). Four-step pathway from phenylpyruvate to benzylamine, an intermediate to the high-energy propellant CL-20. ACS Synthetic Biology, 10(9), 2187-2196. https://doi.org/10.1021/acssynbio.1c00021.
10. Silva-Rocha, R., Martínez-García, E., Calles, B., Chavarría, M., Arce-Rodríguez, A., De Las Heras, A., Páez-Espino, A. D., Durante-Rodríguez, G., Kim, J., Nikel, P. I., Platero, R., & De Lorenzo, V. (2013). The Standard European Vector Architecture (SEVA): A coherent platform for the analysis and deployment of complex prokaryotic phenotypes. Nucleic Acids Research, 41(Database issue), D666. https://doi.org/10.1093/NAR/GKS1119.
11. Zhou, Y., Sekar, B. S., Wu, S., & Li, Z. (2020). Benzoic acid production via cascade biotransformation and coupled fermentation-biotransformation. Biotechnology and Bioengineering, 117(8), 2340-2350. https://doi.org/10.1002/bit.27366.
12. Zhou, Y., Wu, S., Mao, J., & Li, Z. (2018). Bioproduction of benzylamine from renewable feedstocks via a nine-step artificial enzyme cascade and engineered metabolic pathways. ChemSusChem, 11(13), 2221-2228. https://doi.org/10.1002/cssc.201800709.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method of producing an amine, wherein the method comprises:
(a) the conversion of a carboxylic acid substrate into an aldehyde catalysed by a carboxylic acid reductase (CAR), wherein the carboxylic acid substate is a carboxylic acid, a conjugate base of a carboxylic acid, a salt of a carboxylic acid or an ester of a carboxylic acid; and
(b) the conversion of the aldehyde into an amine catalysed by a transaminase (TA).

2. The method of claim 1, wherein the carboxylic acid substrate is benzoic acid, benzoate, a salt of benzoic acid or an ester of benzoic acid, wherein the aldehyde is benzaldehyde and wherein the amine is benzylamine.

3. The method of claim 1 or 2, wherein the CAR comprises an amino acid sequence with at least 40% sequence identity to one or more of SEQ ID NO: 1, 2, 3, 4, 5 or 6.

4. The method of any one of claims 1 to 3, wherein the TA is an omega-TA (ω-TA).

5. The method of claim 4, wherein the ω-TA comprises an amino acid sequence with at least 40% sequence identity to one or more of SEQ ID NO: 7, 8, 9, 10, 11, or 25.

6. The method of any one of claims 1 to 5, wherein step (a) also comprises activity by a phosphopantetheinyl transferase (PPTase).

7. The method of claim 6, wherein the PPTase comprises an amino acid sequence with at least 40% sequence identity to SEQ ID NO: 12.

8. The method of any one of claims 1 to 7, wherein step (b) also comprises activity by an alanine dehydrogenase (AlaDH).

9. The method of claim 8, wherein the AlaDH comprises an amino acid sequence with at least 40% sequence identity to SEQ ID NO: 13.

10. The method of any one of claims 1 to 9, wherein an amine donor is provided to facilitate step (b).

11. The method of any one of claims 1 to 10, wherein steps (a) and (b) occur within an organism expressing the recited enzymes.

12. The method of claim 11, wherein the carboxylic acid substrate is provided exogenously to the organism.

13. The method of claim 12, wherein the exogenous carboxylic acid substrate has been produced biologically.

14. The method of any one of claims 11 to 13, wherein the organism has been modified to inhibit the reduction of aldehydes.

15. The method of claim 14, wherein the organism has been modified to reduce or eliminate the activity or expression of one or more aldehyde reductases, aldo-keto reductases (AKRs) and/or alcohol dehydrogenases (ADHs), preferably wherein the one or more enzymes comprise an amino acid sequence with at least 40% sequence identity to one or more of SEQ ID NO: 14, 15, 16, 17, 18 or 19.

16. The method of any one of claims 11 to 15, wherein the organism is a microorganism, preferably a bacterium.

17. The method of claim 16, wherein the carboxylic acid substrate is provided exogenously to the microorganism while the microorganism is in a growth stage.

18. The method of claim 16, wherein the carboxylic acid substrate is provided exogenously to the microorganism while the microorganism is in a resting stage following a growth stage.

19. The method of any one of claims 1 to 10, wherein each of the recited enzymes is either comprised within a cell lysate or is present in a purified form.

20. The method of any one of claims 1 to 19, wherein the method further comprises the step:
(c) isolating the produced amine.

21. A method of producing a compound, wherein the method comprises:
(a) the method of producing an amine according to any previous claim; and
(b) the production of a compound utilising the amine produced in step (a),
preferably wherein the amine produced in step (a) is benzylamine and the compound produced in step (b) is hexanitrohexaazaisowurtzitane (CL-20).

22. An organism for producing an amine, wherein the organism expresses a heterologous CAR and a heterologous TA.

23. The organism of claim 22, wherein the organism further expresses a PPTase and/or an AlaDH.

24. The organism of claim 22 or 23, wherein the organism has been modified to inhibit the reduction of aldehydes, preferably wherein the organism has been modified to reduce or eliminate the activity or expression of one or more aldehyde reductases, AKRs and/or ADHs.

25. The organism of any one of claims 22 to 24, wherein the organism is a microorganism, preferably a bacterium.
